(19) United States
(12) Patent Application Publication
(10) Pub. No.: US 2014/0228243 A1
(43) Pub. Date: Aug. 14, 2014

(54) PROGNOSTIC METHODS AND COMPOSITIONS FOR PREDICTING INTERFERON TREATMENT EFICACY IN A SUBJECT

(71) Applicant: Yoav Smith, Jerusalem (IL)

(72) Inventor: Yoav Smith, Jerusalem (IL)

(73) Assignee: YISSUM RESEARCH DEVELOPMENT COMPANY OF THE HEBREW UNIVERSITY OF JERUSALEM, Jerusalem (IL)

(21) Appl. No.: 14/342,603

(22) PCT Filed: Sep. 3, 2012
(86) PCT No.: PCT/IL2012/050345

§ 371 Date: Mar. 4, 2014

Related U.S. Application Data
(60) Provisional application No. 61/530,975, filed on Sep. 4, 2011.

(51) Int. Cl. C12Q 1/68

(52) U.S. Cl. CPC C12Q 1/6881
USPC 506/9; 435/6.11; 435/6.12

(57) ABSTRACT

The present invention relates to methods, compositions and kits for predicting, assessing and evaluating responsiveness and success of interferon treatment as well as methods for monitoring disease progression and pathophysiology in a subject treated with interferon, using miR-146a and optionally at least one of miR-146a regulated genes as biomarkers.

### TECHNOLOGICAL FIELD

The invention relates to personalized medicine. More specifically, the invention relates to methods, compositions and kits for predicting, assessing and evaluating responsiveness and success of interferon treatment as well as methods for monitoring disease progression and pathophysiology in a subject treated with interferon.

### BACKGROUND

Interferon therapy is widely used in the treatment of a variety of diseases including for example, multiple sclerosis (MS), hepatitis B, hepatitis C, inflammatory diseases and many cancers types. However, not all subjects treated with interferon equally respond to this therapy and moreover, responsive subjects experience relapse of the disease after remission periods. In fact, in both MS and type 1 hepatitis C Virus (HCV) the success of treatment is only about 50%, namely about half of the patients administered with interferon will not benefit but rather experience only related side effects.

Evaluating the differences in the genetic profile of the two groups of patients can provides valuable insight in the interferon resistant mechanism.
Chen et al. 2005, compared the gene expression levels in liver specimens taken before treatment from 15 non-responders and 16 responders to Pegylated interferon (IFN-alpha), identified 18 genes that have a significantly different expression between all responders and all non-responders and concluded that up-regulation of a specific set of interferon-responsive gens predict non response to exogenous treatment.
Taylor M, et al. 2007, found that the induced levels of known interferon-stimulated genes such as the OAS1, OAS2, MX1, IRF-7 and TLR-7 genes is lower in poor-response patients than in marked- or intermediate-response patients.
Van Baarsen et al., 2008 show that the expression level of interferon response genes in the peripheral blood of multiple sclerosis patients prior to treatment can serve a role as a biomarker for the differential clinical response to interferon beta.
Zeremaki M, et al., 2007 showed that PEG-interferon induced elevations in IP-10 are greater in responders than in non-responders after the first PEG-interferon dose.
Tarantino et al., 2008 described that serum levels of B-Lymphocyes stimulator (BLyS) have a potential role as a predictor of outcome in patients with acute hepatitis C.

The Inventor previous US Patent Application, US2009157324 describes a computational method for selecting a group of genes from a predetermined group of genes whose expression level is significantly different among a first group of individuals (being for example responders to a treatment) and comparing their expression in a second group of individuals (for example not responders). The statistical significance of each group of genes is determined in both up regulated genes or down regulated genes, namely their expression in the first group is higher or lower than in the second group, respectively. The genes in both groups (up regulated and down regulated) are ranked according to number of times each gene was ranked in the highest statistical significant score. A subset of genes having the highest score, either up regulated or down regulated are then selected as biomarkers.

In another Application by the Inventor, International Patent Publication WO10076788, computational and experimental methods are provided for predicting the responsiveness of a subject to interferon therapy by measuring the expression level of various genes such as OAS3, IF16, ISG15, OAS2, IFIT1, KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G and IF127. Specifically, the inventor has found that OAS3, IF16, ISG15, OAS2 and IFIT1 are up-regulated in patients that do not respond to interferon treatment as compared to patients that respond to interferon therapy or compared to healthy controls.

MicroRNAs (miRNAs) are a family of regulatory short non-coding RNAs that function by modulating protein production (Williams, 2008). For example, miR-146a is an immediate early-response gene induced by various microbial components and pro-inflammatory mediators that was found to be a NF-kappaB-dependent gene (Taganov et al., 2006). Recent studies have shown that miRNAs can serve as biomarkers for different human diseases.

Thus, new suitable biomarkers, including miRNA molecules needs to be considered for predicting response to therapy, predicting treatment success and monitoring disease prognosis and pathogenesis, specifically chances for disease relapse.

### GENERAL DESCRIPTION

According to a first aspect, the invention relates to a prognostic method for predicting, assessing and monitoring responsiveness of a mammalian subject to interferon treatment. In certain embodiments, the method of the invention comprises the steps of: First, step (a) involves determining the level of expression of miR-146a and optionally of at least one of miR-146a regulated genes in a biological sample of said subject to obtain an expression value. The second step (b) involves comparing the expression value obtained in step (a) to a predetermined standard expression value, or cutoff value. Alternatively, the expression value may be compared to an expression value of miR146a and optionally of at least one of miR-146a regulated genes in at least one control sample. Such control sample may be a sample obtained from at least one of a healthy subject, a subject suffering from an immune-related disorder, a subject that responds to interferon treatment, a non-responder subject, a subject in remission and a subject in relapse. The method of the invention thereby enables predicting assessing and monitoring responsiveness of a mammalian subject to interferon treatment.

In yet further alternative specific embodiments, the second step (b) of the method of the invention involves calculating and determining if the expression value obtained in step (a) is any one of, positive, negative or equal to a predetermined standard expression value, or cutoff value.

A second aspect of the invention relates to a prognostic composition comprising:

(a) detecting molecules specific for determining the level of expression of miR-146a in a biological sample; and
(b) detecting molecules specific for determining the level of expression of at least one of miR-146a regulated genes in a biological sample. In an optional embodiment, the detecting molecules of (a) and (b) may be attached to a solid support.

In yet another aspect, the invention provides a kit comprising: (a) detecting molecules specific for determining the level of expression of miR-146a in a biological sample; and (b) detecting molecules specific for determining the level of expression of at least one of miR-146a regulated genes in a biological sample. In certain embodiments, the kit of the invention may optionally further comprise at least one of:

(c) pre-determined calibration curve providing standard expression values of at least one of miR-146a and of at least one of miR-146a regulated genes; and (d) at least one control sample.

According to another aspect, the invention provides a method for treating, preventing, ameliorating or delaying the onset of an immune-related disorder in a subject. More specifically, the method of the invention may comprise the step of: (a) predicting, assessing and monitoring responsiveness of the tested subject to interferon treatment according to the method of the invention; and (b) selecting an interferon treatment regimen based on said responsiveness thereby treating said subject.

In still a further aspect, the invention provides a method for treating, preventing, ameliorating or delaying the onset of an immune-related disorder in a subject treated with interferon by modulating the expression of miR-146a, the method comprising the step of administering to said subject a therapeutically effective amount of any one of: (a) antisense specific for miR-146a; (b) siRNA specific for miR-146a; and (c) miR-146a oligonucleotide.

In more specific embodiments, where down-regulation of miR-146a is desired, antisense specific for miR-146a or siRNA specific for miR-146a may be applied. Alternatively, where up-regulation of miR-146a is preferred, miR-146a oligonucleotide may be applied.

These and other aspects of the invention will become apparent by the hand of the following drawings.

### BRIEF DESCRIPTION OF THE DRAWINGS

In order to understand the disclosure and to see how it may be carried out in practice, embodiments will now be described, by way of non-limiting example only, with reference to the accompanying drawings, in which:

FIG. 1. is a simplified volcano plot showing the significant changes in the expression level of different genes in peripheral blood mononuclear cells (PBMC) from multiple sclerosis (MS) patients treated for three months with interferon. Expression data was downloaded from the Gene Expression Accession No. GSE26104. The “X”-axis represents log 2 of ratio between gene expression measured after 3 month and a baseline level of the same gene measured before treatment, the points present to the right of the right vertical line (shown at a value of 1 on the x-axis), represent genes that were up regulated by more than 2 folds and the points present to the left of this line represent down regulated genes (appear with negative values). The “Y” axis shows the p value assigned to each point. The horizontal line corresponds to p-value of 0.05, with points above this line correspond to a p values lower than 0.05 (namely, more significant). Abbreviations: val. (value); rat. (ratio).

FIG. 2. is a graph showing miR-146a expression measured in PBMCs of MS patients and of healthy volunteers. Expression data was downloaded from the Gene Expression Omnibus Accession No. GSE17846. The “X”-axis represents the subject number, where numbers 1 to 20 correspond to MS patients and numbers 21 to 41 correspond to healthy volunteers. The “Y” axis represents the normalized expression level of miR-146a.

FIG. 3. is a volcano graph showing the significant changes in the expression level of different genes in PBMC of MS patients treated with interferon, in a relapse period and while stable (remission). Expression data was downloaded from the Gene Expression Omnibus Accession No. GSE19224. The “X”-axis represents the log 2 of the ratio of each gene expression, with the points present to the left of the left vertical line correspond to genes that are down regulated in patients experiencing a relapse and points present to the right of the right vertical correspond to genes that are up regulated in patients while stable. The “Y” axis shows the p value as in FIG. 1. Abbreviations: val. (value); rat. (ratio).

FIG. 4. is a graph showing miR-146a expression measured in multiple melanoma (MM) patients. Expression data was downloaded from the Gene Expression Omnibus Accession No. GSE20994. The “X” axis represents the subject number, with numbers 1 to 22 corresponding to healthy volunteers and numbers 23 to 57 correspond to MM patients. The “Y” axis represents the measured miR-146a expression level.

FIG. 5. is a volcano graph showing the changes in the expression level of different genes measured in patients diagnosed with Hepatitis C virus (HCV), one week before and one week after interferon treatment. Expression data was downloaded from the Gene Expression Omnibus Accession Nos. GSE11190 and GSE17183. The “X”-axis represents the log 2 expression of each gene as in FIG. 3. The “Y” axis shows the p value as in FIG. 1. The horizontal line corresponds to p-value of 0.05, with points above this line correspond to a p values lower than 0.05 (namely, more significant). Abbreviations: val. (value); rat. (ratio).

FIGS. 6A-6C. are volcano plots showing the significant changes in the expression level of different genes measured one hour (FIG. 6A) and six hours post-infection with H5N1 virus in vitro (FIG. 6B) and six hours post-infection with H1N1 virus in vitro (FIG. 6C) [_]. Expression data was downloaded from the Gene Expression Omnibus Accession No. GSE18816. The X axis and the Y axis are as described in FIG. 3. Abbreviations: val. (value); rat. (ratio).

### DETAILED DESCRIPTION OF EMBODIMENTS

Predicting the chances of a patient to respond to treatment before initiation of treatment or at early stages after initiation of treatment is highly valuable and clinically desired. The importance of adjusting suitable treatment protocols is appreciated in view of the fact that a large number of treatment protocols are often associated with some extent of undesired side effects. Thus, predicting response of a patient to a treatment protocol before and/or at early stages after initiation of treatment and/or throughout or after a treatment period may avoid inadequate treatments and reduce unnecessary side effects.

In addition, even if a patient responds to a specific treatment and experiences a remission period, it is not necessarily that the disease will not relapse at some later stages. Thus, identifying breakthrough points throughout the disease and even after remission can asses in predicting the probability of a disease relapse, which has proved to be one of the key for successful treatment of patients.

Interferon is widely clinically used for treatment of a variety of diseases including for example autoimmune diseases such as multiple sclerosis, different types of proliferative disorders and inflammatory diseases such as hepatitis C. Significant therapeutic advances were made in the treatment of interferon associated diseases however, it is still difficult to determine at the time of disease diagnosis and treatment adjustments, which patients will respond to treatment and which would eventually relapse. Surprisingly, although interferon is considered as a state of art therapy in treatment of these diseases, many of the treated patients do not respond to the therapy and even if they do, many of the patients experience a relapse of the disease.

Thus, there is a critical need for reliable predictors that will provide gaudiness and identification of treatment success and failure, breakthrough point and predict inadequate treatments. In addition, responsiveness predictions provided throughout or after treatment periods enable development of alternatives dosing regimens of interferon.

In the present invention, the inventor has used computational tools and identified an arsenal of genes that is differently expressed in patients that were found to respond to interferon treatment and in patients that were found non-responders. In addition, this group of genes was also found to be differently expressed at different stages of disease, namely during relapse of the disease.

Specifically, as shown in Example 1 herein, the inventor has found that expression of miR-146a regulated genes, IFI44L, MX2, RSAD2, IFIT5, IFITM1, IFITM3, IRF7, ISG15, IF127, TRAF6, IF144, IFIT3, OASL, TRIM22, IFIT1, IRAK1 and IRAK2 was up regulated after interferon treatment (compared to a baseline level measured before treatment) in multiple sclerosis patients that were found responsive to interferon treatment. In addition, as shown in Example 3 herein, the expression of the above mentioned miR-146a regulated genes, was found to be down regulated in patients experiencing relapse of multiple sclerosis compared to when stable. Further, as shown in Examples 2 and 4, differences in the expression of miR-146a were observed between cohorts of patients diagnosed with MS or melanoma compared with control healthy individuals.

The inventors have therefore concluded that the identified genes described herein are suitable for predicting, assessing and monitoring response of a patient to interferon treatment.

Thus, according to a first aspect, the invention relates to a prognostic method for predicting, assessing and monitoring responsiveness of a mammalian subject to interferon treatment.

In certain embodiments, the method of the invention comprises the steps of:

First, step (a) involves determining the level of expression of miR-146a and optionally of at least one of miR-146a regulated genes in a biological sample of said subject to obtain an expression value. The second step (b) involves comparing the expression value obtained in step (a) to a predetermined standard expression value, or cutoff value. Alternatively, the expression value may be compared to an expression value of miR146a and optionally of at least one of miR-146a regulated genes in at least one control sample.

Such control sample may be a sample obtained from at least one of a healthy subject, a subject suffering from an immune-related disorder, a subject that responds to interferon treatment, a non-responder subject, a subject in remission and a subject in relapse. The method of the invention thereby enables predicting assessing and monitoring responsiveness of a mammalian subject to interferon treatment. In yet further alternative specific embodiments, the second step (b) of the method of the invention involves calculating and determining if the expression value obtained in step (a) is any one of, positive, negative or equal to a predetermined standard expression value, or cutoff value.

It should be appreciated that, as used herein the term “miR-146a” relates to human MicroRNAs 146a (MiRNA-146a, MIRN146; MIRN146A; miR-146a; miRNA146A) and unless otherwise specifically indicated, refer to microRNA-146a including miR-146a, pre-miR-146a and mature miR-146a. The sequences for mature miR-146a MIMAT0000449 and pre-miR-146a MI0000477 are provided herein in SEQ ID NOs:1 and 2 respectively. The sequences of cDNA of mature miR-146a and pre-miR-146a (NCBI Reference Sequence NR_{—}029701) are provided herein in SEQ ID NOs: 3 and 4 respectively. The sequence of miR-146a primary transcripts corresponding to accession number: EU 147785; is provided herein as SEQ ID NO: 5. An intragenic miR-146a gene corresponding to accession number: DQ658414; is provided herein as SEQ ID NO: 6. As appreciated, intragenic miRNA genes are generally believed to be co-transcribed with their host genes.

“MicroRNAs” (“miRNAs” or “miRs”) as used herein are post-transcriptional regulators that bind to complementary sequences in the three prime untranslated regions (3′ UTRs) of target messenger RNA transcripts (mRNAs), usually resulting in gene silencing. miRNAs are short ribonucleic acid (RNA) molecules, on average only 22 nucleotides long. The human genome may encode over 1000 miRNAs, which may target about 60 percent of mammalian genes and are abundant in many human cell types. Each miRNA may repress hundreds of mRNAs. miRNAs are well conserved in eukaryotic organisms and are thought to be a vital and evolutionarily ancient component of genetic regulation. miRNA genes are usually transcribed by RNA polymerase II (Pol II). The polymerase often binds to a promoter found near the DNA sequence encoding what will become the hairpin loop of the pre-miRNA. The resulting transcript is capped with a specially-modified nucleotide at the 5′ end, polyadenylated with multiple adenosines (a poly(A) tail), and spliced. The product, called a primary miRNA (pri-miRNA), may be hundreds or thousands of nucleotides in length and contain one or more miRNA stem loops. When a stem loop precursor is found in the 3′ UTR, a transcript may serve as a pri-miRNA and a mRNA. RNA polymerase III (Pol III) transcribes some miRNAs, especially those with upstream Alu sequences, transfer RNAs (tRNAs), and mammalian wide interspersed repeat (MWIR) promoter units.

A single pri-miRNA may contain from one to six miRNA precursors. These hairpin loop structures are composed of about 70 nucleotides each. Each hairpin is flanked by sequences necessary for efficient processing. The double-stranded RNA structure of the hairpins in a pri-miRNA is recognized by a nuclear protein known as DiGeorge Syndrome Critical Region 8 (DGCR8 or “Pasha” in invertebrates), named for its association with DiGeorge Syndrome. DGCR8 associates with the enzyme Drosha, a protein that cuts RNA, to form the “Microprocessor” complex. In this complex, DGCR8 orients the catalytic RNase III domain of Drosha to liberate hairpins from pri-miRNAs by cleaving RNA about eleven nucleotides from the hairpin base (two helical RNA turns into the stem). The resulting hairpin, known as a pre-miRNA, has a two-nucleotide overhang at its 3′ end; it has 3′ hydroxyl and 5′ phosphate groups. Pre-miRNAs that are spliced directly out of introns, by passing the Microprocessor complex, are known as “mirtrons.” Originally thought to exist only in Drosopila and C. elegans, mirtrons have now been found in mammals.

Perhaps as many as 16 percent of pri-miRNAs may be altered through nuclear RNA editing. Most commonly, enzymes known as adenosine deaminases acting on RNA (ADARs) catalyze adenosine to inosine (A to I) transitions. RNA editing can halt nuclear processing (for example, of pri-miR-142, leading to degradation by the ribonuclease Tudor-SN) and alter downstream processes including cytoplasmic miRNA processing and target specificity (e.g., by changing the seed region of miR-376 in the central nervous system). Pre-miRNA hairpins are exported from the nucleus in a process involving the nucleocytoplasmic shuttle Exportin-5. In the cytoplasm, the pre-miRNA hairpin is cleaved by the RNase III enzyme Dicer. This endoribonuclease interacts with the 3′ end of the hairpin and cuts away the loop joining the 3′ and 5′ arms, yielding an imperfect miRNA:miRNA* duplex about 22 nucleotides in length. Overall hairpin length and loop size influence the efficiency of Dicer processing, and the imperfect nature of the miRNA:miRNA* pairing also affects cleavage. Although either strand of the duplex may potentially act as a functional miRNA, only one strand is usually incorporated into the RNA-induced silencing complex (RISC) where the miRNA and its mRNA target interact.

The mature miRNA is part of an active RNA-induced silencing complex (RISC) containing Dicer and many associated proteins. RISC is also known as a microRNA ribonucleoprotein complex (miRNP); RISC with incorporated miRNA is sometimes referred to as “miRISC.”

The prefix “mir” is followed by a dash and a number, the latter often indicating order of naming. For example, mir-123 was named and likely discovered prior to mir-456. The uncapitalized “mir-” refers to the pre-miRNA, while a capitalized “miR-” refers to the mature form. miRNAs with nearly identical sequences bar one or two nucleotides are annotated with an additional lower case letter. For example, miR-123a would be closely related to miR-123b. miRNAs that are 100 percent identical but are encoded at different places in the genome are indicated with additional dash-number suffix. miR-123-1 and miR-123-2 are identical but are produced from different pre-miRNAs. Species of origin is designated with a three-letter prefix, e.g., hsa-miR-123 would be from human (Homo sapiens). MicroRNAs originating from the 3′ or 5′ end of a pre-miRNA are denoted with a −3p or −5p suffix. When relative expression levels are known, an asterisk following the name indicates an miRNA expressed at low levels relative to the miRNA in the opposite arm of a hairpin. For example, miR-123 and miR-123* would share a pre-miRNA hairpin, but relatively more miR-123 would be found in the cell.

Human miR-146a is located in the second exon of LOC285628 gene on the human chromosome 5. LOC285628 consists of two exons separated by a long ˜16 kb long intron and is most probably a non-coding RNA gene, since it does not contain a long, continuous open reading frame. The miRNA-146a has been recently shown to be a modulator of differentiation and function of cells of the innate as well as adaptive immunity In addition, the expression of miR-146a was also found to be dysregulated in different types of tumors.

The term “miR-146a regulated genes” as used herein relates to a group of genes being regulated by miR-146a. The expression of miR-146a regulated gens can be negatively proportional to the expression of miR-146a, namely an up regulation of miR-146a may induce a down regulation of the miR-146a regulated genes. Alternatively up regulation of miR-146a may induce an up regulation of the miR-146a regulated genes. The miR146 regulated genes will be described in more detail herein after.

More specifically, “down-regulation” of the miR-146a regulated genes as a result of miR146a expression includes any “decrease”, “inhibition”, “moderation”, “elimination” or “attenuation” in the expression of said genes and relate to the retardation, restraining or reduction of miR-146a regulated genes expression or levels by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

Alternatively, “up-regulation” of the miR-146a regulated genes as a result of miR146a expression includes any “increase”, “elevation”, “enhancement” or “elevation” in the expression of said genes and relate to the enhancement and increase of at least one of miR-146a regulated genes expression or levels by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

It should be noted that in certain embodiments, the expression level of miR-146a and optionally of at least one of miR-146a regulated genes may be determined prior to interferon treatment, during treatment or after interferon treatment.

The prognostic method of the invention is based on measuring and determining the expression level of miR-146a and optionally of at least one of miR-146a regulated genes, in a biological sample.

The terms “level of expression” or “expression level” are used interchangeably and generally refer to a numerical representation of the amount (quantity) of a polynucleotide which may be miRNA or a gene regulated by miRNA or an amino acid product or protein in a biological sample.

“Expression” generally refers to the process by which gene-encoded information is converted into the structures present and operating in the cell. For example, miRNA expression values measured in Real-Time Polymerase Chain Reaction, sometimes also referred to as RT-PCR or quantitative PCR (qPCR), represent luminosity measured in a tested sample, where an intercalating fluorescent dye is integrated into double-stranded DNA products of the qPCR reaction performed on reverse-transcribed sample RNA, i.e., test sample RNA converted into DNA for the purpose of the assay. The luminosity is captured by a detector that converts the signal intensity into a numerical representation which is said expression value, in terms of miRNA. Therefore, according to the invention “expression” of a gene, specifically, a gene encoding miR-146a may refer to transcription into a polynucleotide. Similarly, a gene encoding miR-146a regulated genes may refer to transcription into a polynucleotide translation into a protein, or even posttranslational modification of the protein. Fragments of the transcribed polynucleotide, the translated protein, or the post-translationally modified protein shall also be regarded as expressed whether they originate from a transcript generated by alternative splicing or a degraded transcript, or from a post-translational processing of the protein, e.g., by proteolysis. Methods for determining the level of expression of the biomarkers of the invention will be described in more detail herein after.

In certain and specific embodiments, the method of the invention further comprises an additional and optional step of normalization. According to this embodiment, in addition to determination of the level of expression of miR-146a and optionally of at least one of the biomarkers of the invention, specifically, the miR-146a regulated genes, the level of expression of at least one suitable control reference gene or miRNA (e.g., hoskeeping genes or control miRs) is being determined in the same sample. According to such embodiment, the expression level of the biomarkers of the invention (miR-146a and optionally of at least one of miR-146a regulated genes) obtained in step (a) is normalized according to the expression level of said at least one reference control gene or miR obtained in the additional optional step in said test sample, thereby obtaining a normalized expression value. Optionally, similar normalization is performed also in at least one control sample or a representing standard when applicable. The next step involves comparing the normalized expression value of miR-146a and optionally of at least one of miR-146a regulated genes in the test biological sample obtained in this additional step, with a predetermined standard expression value, or a cut-off value, or with a normalized expression value of miR-146a and optionally of at least one of miR-146a regulated genes in a control sample.

The term “expression value” refers to the result of a calculation, that uses as an input the “level of expression” or “expression level” obtained experimentally and by normalizing the “level of expression” or “expression level” by at least one normalization step as detailed herein, the calculated value termed herein “expression value” is obtained.

More specifically, as used herein, “normalized values” are the quotient of raw expression values of marker genes, namely, miR-146a and at least one of miR-146a regulated genes, divided by the expression value of a control reference gene from the same sample, such as a stably-expressed housekeeping control gene or mirRNA. Any assayed sample may contain more or less biological material than is intended, due to human error and equipment failures. Importantly, the same error or deviation applies to both the marker genes of the invention and to said control reference gene or mirRNAS, whose expression is essentially constant. Thus, division of the marker gene raw expression value (namely, miR-146a and at least one of miR-146a regulated genes) by the control reference mirRNA or gene raw expression value yields a quotient which is essentially free from any technical failures or inaccuracies (except for major errors which destroy the sample for testing purposes) and constitutes a normalized expression value of said marker gene. This normalized expression value may then be compared with normalized cutoff values, i.e., cutoff values calculated from normalized expression values. In certain embodiments, the control reference gene or miRNA could be 5S ribosomal RNA (rRNA), U6 small nuclear RNA, or any microRNA that maintains stable in all samples analyzed in the microarray analysis. The expression level of each miRNA relative to 5S may be determined by using 2-dCt method, where dCt=(Ct miRNA-Ct 5S rRNA). The relative expression may be calculated automatically by the LightCycler software. The Ct (cycle threshold) is defined as the number of amplification cycles required for the fluorescent signal to cross the threshold (i.e. exceeds background level). Ct levels are inversely proportional to the amount of target nucleic acid in the sample (i.e. the lower the Ct level the greater the amount of target nucleic acid in the sample).

In other embodiments, the miRXplore Universal Reference (UR) may be used as control reference, representing a pool of 979 synthetic miRNA for comparison of multiple samples.

Normalized miR-146a and at least one of miR-146a regulated genes expression level values that are higher (positive) or lower (negative) in comparison with a corresponding predetermined standard expression value or a cut-off value in a control sample predict to which population of patients the tested sample belongs.

It should be appreciated that an important step in the prognostic method of the inventions is determining whether the normalized expression value of any one of miR-146a and at least one of miR-146a regulated genes is changed compared to a pre determined cut off.

The second step of the method of the invention involves comparing the expression values determined for the tested sample with predetermined standard values or cutoff values, or alternatively, with expression values of a control sample. As used herein the term “comparing” denotes any examination of the expression level and/or expression values obtained in the samples of the invention as detailed throughout in order to discover similarities or differences between at least two different samples. It should be noted that comparing according to the present invention encompasses the possibility to use a computer based approach. In yet more specific embodiments, the second step (b) of the method of the invention involves calculating and determining if the expression value obtained in step (a) is any one of, positive, negative or equal to a predetermined standard expression value, or cutoff value. Such step involves calculating and measuring the difference between the expression values of the examined sample and the cutoff value and determining whether the examined sample can be defined as positive or negative.

As described hereinabove, the method of the invention refers to a predetermined cutoff value. It should be noted that a “cutoff value”, sometimes referred to simply as “cutoff” herein, is a value that meets the requirements for both high diagnostic sensitivity (true positive rate) and high diagnostic specificity (true negative rate).

It should be noted that the terms “sensitivity” and “specificity” are used herein with respect to the ability of one or more markers, specifically miR-146a and optionally, at least one of miR-146a regulated genes, to correctly classify a sample as belonging to a pre-established population associated with responsiveness to treatment or to a specific relapse rate.

“Sensitivity” indicates the performance of the bio-markers of the invention, the miR-146a and optionally, at least one of miR-146a regulated genes, with respect to correctly classifying samples as belonging to pre-established populations that are likely to respond to therapy or to relapse, wherein said bio-markers are consider here as miR-146a and at least one of miR-146a regulated genes.

“Specificity” indicates the performance of the bio-markers of the invention with respect to correctly classifying samples as belonging to pre-established populations that are likely to respond or unlikely to relapse.

Simply put, “sensitivity” relates to the rate of correct identification of responsiveness and high-relapse rate samples as such out of a group of samples, whereas “specificity” relates to the rate of correct identification of lack of responsiveness and low-relapse rate samples as such out of a group of samples. Cutoff values may be used as a control sample, said cutoff values being the result of a statistical analysis of miRNAs and miR-regulated genes expression values differences in pre-established populations healthy, responsive, nonresponsive, relapsed or remained disease-free (remission).

Thus, a given population having specific clinical parameters will have a defined likelihood to respond to relapse based on the expression values of miR-146a and optionally of at least one of miR-146a regulated genes being above or below said cutoff values. It should be emphasized that the nature of the invention is such that the accumulation of further patient data may improve the accuracy of the presently provided cutoff values, which are based on an ROC (Receiver Operating Characteristic) curve generated according to said patient data using, for example, the analytical software program developed by the inventor. The miR-146a and at least one of miR-146a regulated genes expression values are selected along the ROC curve for optimal combination of prognostic sensitivity and prognostic specificity which are as close to 100 percent as possible, and the resulting values are used as the cutoff values that distinguish between patients who will relapse at a certain rate, and those who will not (with said given sensitivity and specificity). Similar analysis may be performed when responsiveness to interferon treatment is being examined to distinguish between responsive and non-responsive subjects. The ROC curve may evolve as more and more patient-responsiveness and relapse data and related miR-146a and miR-146a related gene expression values are recorded and taken into consideration, modifying the optimal cutoff values and improving sensitivity and specificity. Thus, the provided cutoff values should be viewed as a starting point that may shift as more patient-relapse, or responder and non-responder data allows more accurate cutoff value calculation. Although considered as initial cutoff values, the presently provided values already provide good sensitivity and specificity, and are readily applicable in current clinical use, even in patients undergoing different treatment regimens.

As noted above, the expression value determined for the examined sample (or the normalized expression value) is compared with a predetermined cutoff or a control sample. More specifically, in certain embodiments, the expression value obtained for the examined sample is compared with a predetermined standard or cutoff value. In further embodiments, the predetermined standard expression value, or cutoff value has been pre-determined and calculated for a population comprising at least one of healthy subjects, subjects suffering from an immune-related disorder, subjects that respond to interferon treatment, non-responder subjects, subjects in remission and subjects in relapse.

Still further, in certain alternative embodiments where a control sample is being used (instead of, or in addition to, pre-determined cutoff values), the normalized expression values of miR146a and at least one of miR-146a regulated genes used by the invention in the test sample are compared to the expression values in the control sample. In certain embodiments, such control sample may be obtained from at least one of a healthy subject, a subject suffering from an immune-related disorder, a subject that responds to interferon treatment, a non-responder subject, a subject in remission and a subject in relapse.

In certain specific embodiments, the method of the invention may be specifically applicable for predicting responsiveness of a mammalian subject to interferon treatment. In such case, the method may comprise the steps of:

First (a), determining the level of expression of miR-146a and optionally of at least one of miR-146a regulated genes in at least one biological sample of the examined subject to obtain an expression value. In the second step (b), the expression value obtained in step (a) is compared with a predetermined standard expression value or cutoff value, thereby predicting responsiveness of a mammalian subject to interferon treatment. Alternatively, the expression value obtained for the examined sample may be compared with the expression value of miR146a and optionally of at least one of miR-146a regulated genes in at least one control sample, for example, a healthy, a responder and a non-responder subject. According to such embodiments, the level of expression of miR-146a and optionally of at least one of miR-146a regulated genes in determined is at least one biological sample at any time before initiation of treatment and the obtained expression value is used to predict if the subject will respond to treatment. The expression value may be compared to an expression value of a population of subjects that respond to interferon treatment and/or to an expression value of a population of subjects that do not respond to interferon treatment. In yet further alternative specific embodiments, the second step (b) of the method of the invention involves calculating and determining if the expression value obtained in step (a) is any one of, positive, negative or equal to a predetermined standard expression value, or cutoff value.

Thus, in certain embodiments, a positive expression value, or in other words, a higher expression value of the biomarker of the invention miR146a and optionally of at least one of miR-146a regulated genes, as compared to the predetermined standard expression value (cutoff value), indicates that said subject belongs to a pre-established population associated with lack of responsiveness to interferon treatment and therefore, the subject may be considered as a non-responsive subject.

Alternatively, where the expression value of the examined subject is compared with the expression value of a control sample, for example, a population of subjects that respond to interferon treatment, a positive or higher expression value of the sample, indicates that the examined subject is a non-responsive subject. When the control sample is a population of non-responder subjects, a positive or equal expression value, indicates that the examined subject belongs to a population of subjects that lack of responsiveness.

It should be noted that according to this specific embodiment, for predicting responsiveness, determination of an expression value is performed prior to initiation of interferon treatment.

As used herein the term “predicting responsiveness” refers to determining the likelihood that the subject will respond to interferon treatment, namely the success or failure of interferon treatment.

The term “response” or “responsiveness” to interferon treatment refers to an improvement in at least one relevant clinical parameter as compared to an untreated subject diagnosed with the same pathology (e.g., the same type, stage, degree and/or classification of the pathology), or as compared to the clinical parameters of the same subject prior to interferon treatment.

The term “non responder” to interferon treatment refers to a patient not experiencing an improvement in at least one of the clinical parameter and is diagnosed with the same condition as an untreated subject diagnosed with the same pathology (e.g., the same type, stage, degree and/or classification of the pathology), or experiencing the clinical parameters of the same subject prior to interferon treatment.

As detailed above, the prediction obtained by the method of the invention made by comparing between the sample and the patient population may be dependent on the selection of population of patients to which the sample is compared to. A positive or higher expression value of the sample over a population of responders indicates that the examined subject is a non-responsive subject.

In accordance with some embodiments, a positive expression value (or higher expression) of either miR146a and optionally of at least one of miR-146a regulated genes reflects a high expression of said miRNA and the regulated genes and is therefore indicative of a specific probability of lack of responsiveness to interferon treatment, said probability being higher than the specific probability of responsiveness in patients where the corresponding initial expression value of either miR146a and optionally of at least one of miR-146a regulated genes are negative.

To disambiguate, a positive expression value indicates a higher risk for non-responsiveness to interferon treatment than a negative expression value. More particularly, the lack of responsiveness to interferon treatment is at least 1 percent, at least percent 2, at least 3 percent, at least 3 percent, at least 4 percent, at least 5 percent, at least 6 percent, at least 7 percent, at least 8 percent, at least 9 percent, at least 10 percent, at least 11 percent, at least 12 percent, at least 13 percent, at least 14 percent, at least 15 percent, at least 16 percent, at least 17 percent, at least 18 percent, at least 19 percent, at least 20 percent, at least 21 percent, at least 22 percent, at least 23 percent, at least 24 percent, at least 25 percent, at least 26 percent, at least 27 percent, at least 28 percent, at least 29 percent, at least 30 percent, at least 31 percent, at least 32 percent, at least 33 percent, at least 34 percent, at least 35 percent, at least 36 percent, at least 37 percent, at least 38 percent, at least 39 percent, at least 40 percent, at least 41 percent, at least 42 percent, at least 43 percent, at least 44 percent, at least 45 percent, at least 46 percent, at least 47 percent, at least 48 percent, at least 49 percent, at least 50 percent, at least 51 percent, at least 52 percent, at least 53 percent, at least 54 percent, at least 55 percent, at least 56 percent, at least 57 percent, at least 58 percent, at least 59 percent, at least 60 percent, at least 70 percent, at least 80 percent, at least 90 percent or more higher than the lack of responsiveness of patient population treated with interferon associated with the corresponding negative expression value (that reflects lower initial levels of expression of either miR146a and optionally of at least one of miR-146a regulated genes).

In some embodiments, the term “specific probability” refers to a probability of a patient to respond to interferon treatment based on miR-146a and at least one miR-146a regulated gene expression pattern, wherein the probability is calculated according to the patient population analysis provided herein, but may be further fine-tuned as more patient clinical data is accumulated and the same statistical analysis may be reiterated using the augmented clinical population database.

Examples 2 and 4 herein below provides an example for a predetermined cut-off value of miR-146a expression that may be helpful in differentiating responders and non-responders and thus enable to predict response to interferon treatment, prior to initiation of treatment. High expression values, or “positive” expression values compared to this predetermined cut-off value are indicative of lack of response to treatment, whereas low expression values, or “negative” expression value, compared to this predetermined cut-off value are indicative of response to treatment.

As a specific and non-limiting example, a normalized cut off value in MS patients and melanoma patients of about 300 was determined. Thus, according to the method of the invention, a patient that is diagnosed with a disease such as MS or melanoma and is in need for interferon treatment, is being initially determined for the miR-146a expression value. If the measured expression value of miR-146a is higher than 300, the patient has a probability not to respond to the treatment, visa versa, if the measured expression value of miR-146a is lower than 300, the patient has a high probability to respond to treatment.

In some other embodiments, the normalized cut off value for miR146a expression may be at least about 250, at least about 260, at least about 270, at least about 280, at least about 290, at least about 300, at least about 310, at least about 320, at least about 330, at least about 340, at least about 350, at least about 360, at least about 370, at least about 380, at least about 390, at least about 400, at least about 410, at least about 420, at least about 430, at least about 430, at least about 450, at least about 466, at least about 470, at least about 480 at least about 490 and at least about 500.

As detailed below, it should be appreciated that the cut off value is highly dependent on the size of the tested averaged group as well as the extent of homogeneity and/or heterogeneity of the tested patients. Thus, determination of the cut off value is considered a dynamic computational process that is being iteratively verified and corrected.

As detailed above, the method of the invention is also suitable for following the responsiveness of a patient to treatment at any time point after treatment. Accordingly, the patient may be evaluated in at least one time point after initiation of treatment in order to asses if the treatment protocol is efficient and appropriate. Determination can be carried out at an early time points such that a decision may be made regarding continuation of the treatment or alternatively readjusting the treatment protocol.

Thus, in yet other embodiments, the invention provides a method for assessing responsiveness of a mammalian subject to interferon treatment or evaluating the efficacy of interferon treatment on a subject. This method is based on determining the expression value of the biomarkers of the invention before and after initiation of interferon treatment, and calculating the ratio of the expression as a result of the treatment. The method therefore comprises the step of:

First, in step (a), determining the level of expression of at least one of miR-146a and of at least one of miR-146a regulated genes in a biological sample of the examined subject to obtain an expression value. It should be noted that the sample is obtained prior to initiation of said treatment. The second step (b) involves determining the level of expression of at least one of miR-146a and of at least one of miR-146a regulated genes in at least one other biological sample of said subject, to obtain an expression value in said sample. This at least one other sample is obtained after initiation of said treatment. In the next step (c), calculating the rate of change between the expression value obtained in step (a) before initiation, and the expression value obtained in step (b), after the initiation of the treatment. It should be noted that for determining the rate of change, the ratio between the expression value of a sample obtained after initiation of the treatment, and the expression value of a sample obtained before initiating interferon treatment, is calculated. In certain embodiments, the ratio may be calculated between the expression values of a sample obtained before to the expression value of a sample obtained after initiation of interferon treatment. In the next step (d), the rate of change obtained in step (c) is compared with a predetermined standard rate of change determined between at least one sample obtained prior to and at least one sample obtained following interferon treatment. As an alternative to the use of a predetermined cutoff value of such rate of change, the method of the invention may involve the use of at least one control sample, and the rate of change calculated for the examined subject will be compared to the rate of change calculated for expression values in at least one control sample obtained prior and following interferon treatment.

In yet a further specific embodiments, the fourth step (d) of the method of the invention involves calculating and determining if the rate of change obtained in step (c) is any one of, positive, negative or equal to a predetermined standard rate of change.

It should be noted that at least one of either (i) a negative or equal rate of change of miR-146a expression value or (ii) a positive rate of change in the expression values of at least one of miR-146a regulated genes in said sample as compared to a predetermined standard rate of change (predetermined cutoff of the rate of change), or to the rate of change calculated for expression values in at least one control sample obtained prior and following interferon treatment, indicates that the examined subject belongs to a pre-established population associated with responsiveness to interferon treatment. Such result is therefore indicative of a successful therapy. This method thereby provides assessing responsiveness of a mammalian subject to interferon treatment or evaluating the efficacy of interferon treatment on a subject.

According to such embodiments, the method of the invention further provides a tool for selecting an interferon treatment regimen for treating a subject diagnosed with a condition, by assessing and evaluating the efficacy of interferon treatment given to a subject suffering from condition to be treated, and selecting an interferon treatment regimen based on the evaluation; thereby selecting the treatment regimen for treating the subject diagnosed with a condition.

As used herein the phrase “assessing the responsiveness or evaluating efficacy of interferon treatment” refers to determining the likelihood (predicting) that interferon treatment is efficient or non-efficient in treating a specific condition, e.g., the success or failure of the treatment in treating the condition in a subject in need thereof. The term “efficacy” as used herein refers to the extent to which interferon treatment produces a beneficial result, e.g., an improvement in one or more symptoms of the pathology (caused by the condition to be treated) and/or clinical parameters related to the pathology as described herein below. For example, the efficacy of interferon treatment may be evaluated using standard therapeutic indices for each condition separately being for example, a proliferative disorder, an autoimmune disease or an infectious disease.

According to some embodiments of the invention, the efficacy of interferon treatment is a long-term efficacy. As used herein the phrase “long-term efficacy” refers to the ability of a treatment to maintain a beneficial result over a period of time, e.g., at least about 16 weeks, at least about 26 weeks, at least about 32 weeks, at least about 36 weeks, at least about 40 weeks, at least about 48 weeks, at least about 52 weeks, at least about 18 months, at least about 24 months, at least about 3 years, at least about 4 years, at least about 5 years, at least about 6 years, at least about 7 years, at least about 8 years, at least about 9 years, at least about 10 years, or longer.

According to some embodiments of the invention, a treatment with interferon that either directly or indirectly affects the condition to be treated, is considered efficient in treating a condition if it exerts an improvement in at least one relevant clinical parameter related to said condition in the treated subject as compared to an untreated subject diagnosed with the same condition (e.g., where the condition is cancer, such parameter include the type, stage, degree and/or classification of the solid tumor), or as compared to the clinical parameters related to the said condition of the same subject prior to the interferon treatment.

By obtaining at least two and preferably more biological samples from a subject and analyzing them according to the method of the invention, the prognostic method may be effective for assessing responsiveness to treatment by monitoring molecular alterations indicating a success or failure of treatment in said patient. Thus, the prognostic method of the invention may be applicable for early assessment. Prior as used herein is meant the first time point is at any time before initiation of treatment, ideally several minutes before initiation of treatment. However, it should be noted that any time point before initiation of the treatment, including hours, days, weeks, months or years, may be useful for this method and is therefore encompassed by the invention. The second time point is collected from the same patient after hours, days, weeks, months or even years after initiation of treatment. More specifically, at least 3 hours, at least 4 hours, at least 6 hours, at least 10 hours, at least 12 hours, at least 24 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 40 days, at least 50 days, at least 60 days, at least 70 days, at least 78 days, at least 80, at least 90 days, at least 100 days, at least 110, at least 120 days, at least 130 days, at least 140 days or at least 150 days after initiation of treatment.

In some embodiments, the second time point is obtained between 1 hour to 24 month after initiation of the treatment. In some other embodiments, the second time point is between 1 hour to 6 hours after initiation of the treatment. In yet some other embodiments, the second time point is between 1 month to 3 month after initiation of the treatment.

In practice, for assessing response to interferon treatment, at least two test samples (before and after treatment) must be collected from the treated patient, and preferably more. The expression level of miR-146a and at least one of miR-146a regulated genes is then determined using the method of the invention, applied for each sample. As detailed above, the expression value is obtained from the experimental expression level. The rate of change of each biomarker expression, namely miR-146a and at least one of miR-146a regulated genes is then calculated and determined by dividing the two expression values obtained from the same patient in different time-points or time intervals one by the other.

It should be noted that it is possible to divide the prior-treatment expression value by the after treatment expression value and vise versa. For the sake of clarity, as used herein, the rate of change is referred as the ratio obtained when dividing the expression value obtained at the later time point of the time interval by the expression value obtained at the earlier time point (for example before initiation of treatment).

For example, this interval may be at least one day, at least three days, at least three days, at least one week, at least two weeks, at least three weeks, at least one month, at least two months, at least three months, at least four months, at least five months, at least one year, or even more. Permeably the second point is obtained at the earlier time point that can provide valuable information regarding assessing response of the patient to interferon treatment.

As detailed above, this rate of change calculated for the examined sample is compared with a predetermined standard rate of change. The predetermined standard rate of change may be determined between at least one sample obtained prior to and at least one sample obtained following interferon treatment. It must be recognized that these predetermined rates of change were calculated for populations described herein and therefore reflect the rate in said specific population. As an alternative to the use of a predetermined cutoff value of such rate of change, the method of the invention may involve the use of at least one control samples, and the rate of change calculated for the examined subject will be compared to the rate of change calculated for expression values in at least one control sample obtained prior and following interferon treatment. In yet further alternative specific embodiments, the fourth step (d) of the method of the invention involves calculating and determining if the rate of change obtained in step (c) is any one of, positive, negative or equal to a predetermined standard rate of change.

In accordance with some embodiments, a negative or equal rate of change of miR146a expression value as compared to the predetermined standard rate of change is indicative of a specific probability to respond to interferon treatment, said probability being higher than the specific probability of responsiveness in patients where the corresponding rate of change of miR146a expression value is positive.

Similarly, a positive rate of change in the expression value of at least one of miR-146a regulated genes predetermined standard rate of change is indicative of a specific probability to respond to interferon treatment, said probability being higher than the specific probability of responsiveness in patients where the corresponding rate of change of at least one of miR-146a regulated genes is negative. In contrast, a negative or equal rate of change in the expression value of at least one of the miR146a regulated genes indicates no response to interferon treatment, and more specifically, that the examined subject belongs to a non-responder population.

To disambiguate, a negative or equal rate of change of miR146a expression value and/or positive rate of change in the expression value of at least one of miR-146a regulated genes indicates a higher probability for responsiveness to interferon treatment than a positive rate of change of miR146a expression value and/or equal or negative rate of change in the expression value of at least one of miR-146a regulated genes. More particularly, responsiveness to interferon treatment is at least 1 percent, at least percent 2, at least 3 percent, at least 3 percent, at least 4 percent, at least 5 percent, at least 6 percent, at least 7 percent, at least 8 percent, at least 9 percent, at least 10 percent, at least 11 percent, at least 12 percent, at least 13 percent, at least 14 percent, at least 15 percent, at least 16 percent, at least 17 percent, at least 18 percent, at least 19 percent, at least 20 percent, at least 21 percent, at least 22 percent, at least 23 percent, at least 24 percent, at least 25 percent, at least 26 percent, at least 27 percent, at least 28 percent, at least 29 percent, at least 30 percent, at least 31 percent, at least 32 percent, at least 33 percent, at least 34 percent, at least 35 percent, at least 36 percent, at least 37 percent, at least 38 percent, at least 39 percent, at least 40 percent, at least 41 percent, at least 42 percent, at least 43 percent, at least 44 percent, at least 45 percent, at least 46 percent, at least 47 percent, at least 48 percent, at least 49 percent, at least 50 percent, at least 51 percent, at least 52 percent, at least 53 percent, at least 54 percent, at least 55 percent, at least 56 percent, at least 57 percent, at least 58 percent, at least 59 percent, at least 60 percent, at least 70 percent, at least 80 percent, at least 90 percent or more higher than the lack of responsiveness of patient population treated with interferon associated with the corresponding a negative rate of change of miR146a expression value or positive rate of change in the expression value of at least one of miR-146a regulated genes.

Accordingly, the present invention provides a highly accurate determination of responsiveness as early as at the time of diagnosis, before initiation of treatment, and in fact, may assist in determining the optimal treatment.

As shown in Example 1 provided herein below, in multiple sclerosis patients that were responsive to interferon treatment, a rate of change of at least about two folds was observed in the expression of miR-146a regulated genes measured after 3 month of treatment compared to the baseline value measured before treatment. In non responders the positive rate of change was not observed. Thus, in this specific example, an increase of at least 1.5 in the expression of miR-146a regulated genes when measured for the same patient is indicative for responsiveness. At times, an increase of at least 2, at least 3, at least 4, at least 5, at least 6, at least 7, at least 8 is sufficient to determine responsiveness to treatment.

As appreciated, the predetermined rate of change calculated for a pre-established population as detailed above for example encompasses a range for the rate of change having a low value and a high value, as obtained from a population of individuals including healthy controls, responders and non-responders. Thus a subgroup of responsive patients can be obtained from the entire tested population. In this pre-established responsive population, the low value may be characterized by a low response whereas the high value may be associated with a high response as indicated by regular clinical evaluation. Therefore, in addition to assessing responsiveness to treatment, the rate of change may provide insight into the degree of responsiveness. For example, a calculated rate of change that is closer in its value to the low value may be indicative of a low response and thus although the patient is considered responsive, increasing dosing or frequency of administration may be considered. Alternatively, a calculated rate of change that is closer in its value to the high value may be indicative of a high response, even at times leading to remission and thus lowering the administration dosage may be considered.

For clarity, when referring to a pre-established population associated with responsiveness, it is meant that a statistically-meaningful group of patients treated with interferon was analyzed as disclosed herein, and the correlations between miR-146a and at least one of miR-146a regulated gene expression values (and optionally other patient clinical parameters) and responsiveness to interferon treatment was calculated. For example, a specific fraction of a group of patients, which was found to have a negative rate of change of miR-146a expression value and/or positive rate of change in the expression values of at least one of miR-146a regulated genes over the cutoff values according to the invention, was found to be responsive. Thus, responsiveness is associated with a population expressing low levels of miR-146a that are reduced or remain unchanged in response to interferon, and/or initial low expression levels of at least one of miR-146a regulated genes that are elevated in response to interferon treatment, said population is a pre-established population, that is, a defined population whose responsiveness is known. Moreover, the populations may be defined by miR-146a expression and at least one miR-146a regulated genes vis a vis the cutoff values determined by the invention. The population may optionally be further divided into sub-populations according to other patient parameters, for example gender and age.

The method of the invention may be used for personalized medicine, namely adjusting and customizing healthcare with decisions and practices being suitable to the individual patient by use of genetic information and any additional information collected at different stages of the disease.

In yet another alternative embodiment, for assessing responsiveness of a mammalian subject to interferon treatment or evaluating the efficacy of interferon treatment on a subject suffering from a pathologic condition, the method of the invention may comprise:

(a) determining the level of expression of at least one of miR-146a and of at least one of miR-146a regulated genes in a biological sample of said subject to obtain an expression value, wherein said sample is obtained prior to initiation of said treatment;
(b) determining the level of expression of at least one of miR-146a and of at least one of miR-146a regulated genes in at least one other biological sample of said subject, to obtain an expression value, wherein said at least one other sample is obtained after initiation of said treatment;
(c) comparing the expression value obtained in step (a), with the expression value obtained in step (b), or in yet further alternative specific embodiments, calculating and determining if the expression value obtained in step (a) is any one of, positive, negative or equal to the expression value obtained in step (b).

Wherein a lower or equal expression value of miR-146a and a higher expression value of at least one of miR-146a regulated genes in a sample obtained after initiation of said treatment according to step (b) as compared to the expression value in a sample obtained prior to initiation of said treatment according to step (a), indicates that said subject belongs to a pre-established population associated with responsiveness to interferon treatment.

In accordance with such an embodiment, a patient diagnosed with a disease in need for interferon treatment is examined and a sample is obtained before initiation of treatment, the patient is then treated with interferon according to common treatment protocol and at any time point after treatment an additional sample is obtained from the patient. The second sample may be obtained after at least 3 hours, at least 4 hours, at least 6 hours, at least 10 hours, at least 12 hours, at least 24 hours, at least 1 day, at least 2 days, at least 3 days, at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 40 days, at least 50 days, at least 60 days, at least 70 days, at least 78 days, at least 80, at least 90 days, at least 100 days, at least 110, at least 120 days, at least 130 days, at least 140 days or at least 150 days after initiation of treatment.

The first sample may be analyzed at the time it was obtained from the patient or alternatively may be kept under appropriate conditions for example, under freezing conditions, or as a paraffin embedded sample. The two samples are equally analyzed, optionally at the same time, for determining the expression of miR-146a and of at least one of miR-146a regulated genes. The data obtained as an expression value are compared by normalization of the expression level as detailed herein.

Patient having a “negative” that is a lower or equal expression value of miR-146a and a “positive” that is a higher expression value of at least one of miR-146a regulated genes in a sample obtained after initiation of said treatment as compared to the expression value in a sample obtained prior to initiation of said treatment according to step (a) belong to a pre-established population associated with responsiveness to interferon treatment.

In yet other embodiments, the invention provides a method for monitoring disease progression or early prognosis for disease relapse. According to certain embodiments, said method comprises the steps of:

First (a), determining the level of expression of miR-146a and optionally of at least one of miR-146a regulated genes in a biological sample of said subject to obtain an expression value. The next steps involve (b) repeating step (a) to obtain expression values of at least one of miR-146a and of at least one of miR-146a regulated genes, for at least one more temporally-separated test sample. The rate of change of the expression values of at least one of miR-146a and of at least one of miR-146a regulated genes are then calculated in step (c) between said temporally-separated test samples.

In the next step (d), the rate of change obtained in step (c) is compared with a predetermined standard rate of change (cutoff value) determined for expression value between samples obtained from at least one subject in remission and in relapse following interferon treatment or to the rate of change calculated for expression values in at least one control sample obtained in remission and in relapse following interferon treatment. It should be appreciated that in an alternative embodiment, step (d) of the method of the invention involves calculating and determining if the rate of change obtained in step (c) is any one of, positive, negative or equal to a predetermined standard rate of change.

According to certain embodiments, at least one of either (i) a positive rate of change of miR-146a expression value or (ii) a negative rate of change in the expression values of at least one of miR-146a regulated genes in said sample as compared to a predetermined standard rate (cutoff) of change or to the rate of change calculated for expression values in said at least one control sample, indicates that said subject belongs to a pre-established population associated with relapse, thereby indicating that the examined subject is in relapse.

Thus, according to such embodiments, the method of the invention further provides early prognosis/diagnosis for monitoring disease relapse.

The term “relapse”, as used herein, relates to the re-occurrence of a condition, disease or disorder that affected a person in the past. Specifically, the term relates to the re-occurrence of a disease being treated with interferon.

Prognosis is defined as a forecast of the future course of a disease or disorder, based on medical knowledge. This highlights the major advantage of the invention, namely, the ability to predict relapse rate in patients as soon as they are diagnosed, even prior to treatment, based on a specific genetic fingerprinting of a patient. This early prognosis facilitates the selection of appropriate treatment regimens that may minimize the predicted relapse, individually to each patient, as part of personalized medicine. Thus, the inventor's surprising finding that miR-146a and at least one of miR-146a regulated gene expression correlates with relapse is both novel and extremely useful.

As indicated above, in accordance with some embodiments of the invention, in order to asses response to interferon treatment at least two “temporally-separated” test samples must be collected from the treated patient and compared thereafter in order to obtain the rate of expression change in miR-146a and miR-146a regulated genes. In practice, to detect a change in miR-146a and at least oneR-146a regulated genes expression, at least two “temporally-separated” test samples and preferably more must be collected from the patient.

The expression of at least one of the markers is then determined using the method of the invention, applied for each sample. As detailed above, the rate of change in marker expression is calculated by determining the ratio between the two expression values, obtained from the same patient in different time-points or time intervals.

This period of time, also referred to as “time interval”, or the difference between time points (wherein each time point is the time when a specific sample was collected) may be any period deemed appropriate by medical staff and modified as needed according to the specific requirements of the patient and the clinical state he or she may be in. For example, this interval may be at least one day, at least three days, at least three days, at least one week, at least two weeks, at least three weeks, at least one month, at least two months, at least three months, at least four months, at least five months, at least one year, or even more.

In some embodiments, one of the time points may correspond to a period in which a patient is experiencing a remission of the disease.

The term “remission”, as used herein, relates to the state of absence of disease activity in patients known to have un-curable chronic illness. It is commonly used to refer to absence of active MS or cancer when this disease is expected to manifest again in the future. A partial remission may be defined for cancer as 50 percent or greater reduction in the measurable parameters of tumor growth as may be found on physical examination, radiologic study, or by biomarker levels from a blood or urine test. A complete remission is defined as complete disappearance of all such manifestations of disease. Each disease or even clinical trial can have its own definition of a partial remission. For MS, with symptoms occurring either in discrete episodes (relapsing forms) or slowly accumulating over time (progressive forms), a partial remission may be defined as 50 percent or greater reduction in the intensity and frequency of episodes or attacks.

When calculating the rate of change, one may use any two samples collected at different time points from the patient. To ensure more reliable results and reduce statistical deviations to a minimum, averaging the calculated rates of several sample pairs is preferable. A calculated or average positive rate of change of the expression values of miR-146a and/or negative rate of change of the expression values of at least one of miR-146a regulated genes indicates that the subject is in relapse. It should be noted that in certain embodiments, where normalization step is being performed, the expression values referred to above, are normalized expression values.

As indicated above, in order to execute the prognostic method of the invention, at least two different samples must be obtained from the subject in order to calculate the rate of change in the expression of miR-146a and optionally, of at least one of miR-146a regulated genes. By obtaining at least two and preferably more biological samples from a subject and analyzing them according to the method of the invention, the prognostic method may be effective for predicting, monitoring and early diagnosing molecular alterations indicating a relapse in said patient.

Thus, the prognostic method may be applicable for early, sub-symptomatic diagnosis of relapse when used for analysis of more than a single sample along the time-course of diagnosis, treatment and follow-up.

An “early diagnosis” provides diagnosis prior to appearance of clinical symptoms. Prior as used herein is meant days, weeks, months or even years before the appearance of such symptoms. More specifically, at least 1 week, at least 1 month, 2 months, 3 months, 4 months, 5 months, 6 months, 7 months, 8 months, 9 months, 10 months, 11 months, 12 months, or even few years before clinical symptoms appear.

Simply put, an increase in the expression of miR-146a and a decline in at least one of miR-146a regulated genes indicate a relapse, and may provide an early sign before over symptoms occur, allowing for a quicker and more efficient therapeutic response.

Of course, more samples taken in more time-points may provide a statistically robust analysis of said expression trends, and may also be utilized as a method for continuous monitoring of subjects, especially those still undergoing and those that have undergone therapy. The more samples are available over a given time period, the higher is the resolution of the expression patterns of miR-146a and optionally, the expression of at least one of miR-146a regulated genes during said period.

Also, when data from miR-146a regulated genes is obtained, the most reliable prediction is obtained when a large number of genes share a similar expression profile.

The number of samples collected and used for evaluation of the subject may change according to the frequency with which they are collected. For example, the samples may be collected at least every day, every two days, every four days, every week, every two weeks, every three weeks, every month, every two months, every three months every four months, every 5 months, every 6 months, every 7 months, every 8 months, every 9 months, every 10 months, every 11 months, every year or even more. Furthermore, to assess the trend in expression rates according to the invention, it is understood that the rate of change may be calculated as an average rate of change over at least three samples taken in different time points, or the rate may be calculated for every two samples collected at adjacent time points. It should be appreciated that the sample may be obtained from the monitored patient in the indicated time intervals for a period of several months or several years. More specifically, for a period of 1 year, for a period of 2 years, for a period of 3 years, for a period of 4 years, for a period of 5 years, for a period of 6 years, for a period of 7 years, for a period of 8 years, for a period of 9 years, for a period of 10 years, for a period of 11 years, for a period of 12 years, for a period of 13 years, for a period of 14 years, for a period of 15 years or more. In one particular example, the samples are taken from the monitored subject every two months for a period of 5 years.

A positive rate of change of miR-146a expression value or a negative rate of change in the expression values of at least one of miR-146a regulated genes in said sample as compared to a predetermined standard rate (cutoff) of change or to the rate of change calculated for expression values in said at least one control sample, indicates that said subject belongs to a pre-established population associated with relapse thus indicating that the examined subject is in relapse.

For clarity, when referring to a pre-established population associated with relapse, it is meant that a statistically-meaningful group of patients treated with interferon was analyzed as disclosed herein, and the correlations between the expression level of miR-146a and optionally of at least one of miR-146a regulated gene expression values (and optionally other patient clinical parameters) and relapse rate was calculated. For example, a specific fraction of a group of patients, which was found to have a positive rate of change of miR-146a expression value and/or a negative rate of change in the expression values of at least one of miR-146a regulated genes over the cutoff values according to the invention, was found to relapse in a certain rate. Thus, this rate of relapse is associated with a population expressing high levels of miR-146a or lower expression levels of at least one of miR-146a regulated genes in i.e., said population is a pre-established population, that is, a defined population whose relapse rate is known. Moreover, the populations may be defined by miR-146a expression and at least one miR-146a regulated genes vis a vis the cutoff values of the invention. The population may optionally be further divided into sub-populations according to other patient parameters, for example gender or age.

Nevertheless, the present invention shows that miR-146a and at least one of miR-146a regulated genes may serve as prognostic markers for responsiveness to interferon treatment, specifically for predicting and monitoring relapse in patients treated with interferon. These markers were shown as independent markers that are not affected by clinical parameters or treatment regimen. The expression “associated with a specific relapse rate”, “linked to a specific relapse rate” or “associated with a relapse rate” or similar expressions refer to a statistical connection between the expression values of miR-146a (and optionally, the expression value of at least one of miR-146a regulated genes), the clinical parameters and a specific relapse rate, or the patient population which is known to relapse in that rate.

The method for monitoring disease progression or early prognosis for disease relapse as detailed herein may be used for personalized medicine, by collecting at least two samples from the same patient at different stages of the disease.

Thus, in yet another alternative embodiment for monitoring disease progression or early prognosis of disease relapse on a subject suffering from a condition, the method of the invention may comprise:

(a) determining the level of expression of at least one of miR-146a and of at least one of miR-146a regulated genes in a biological sample of said subject to obtain an expression value, wherein said sample is obtained at any time point after initiation of said treatment;
(b) determining the level of expression of at least one of miR-146a and of at least one of miR-146a regulated genes in at least one other biological sample of said subject, to obtain an expression value, wherein said at least one other sample is obtained at a different time point after initiation of said treatment;
(c) comparing the expression value obtained in step (a), with the expression value obtained in step (b); or calculating and determining if the expression value obtained in step (b) is any one of, positive, negative or equal to the expression value obtained in (a). Wherein a higher (positive) or equal expression value of miR-146a and a lower (negative) expression value of at least one of miR-146a regulated genes in a sample obtained at a later time point after initiation of the treatment according to step (b) as compared to the expression value in a sample obtained at an earlier time point after initiation of said treatment according to step (a), indicates that said subject may be considered in a relapse.

In any case, an increase in the normalized expression values of miR-146a and a reduction in the moralized expression value of at least one of miR-146a regulated genes indicates a relapse, alternatively, a decrease in the normalized expression values of miR-146a and an increase in the moralized expression value of at least one of miR-146a regulated genes may indicate an improvement in the clinical condition of the subject, i.e., that the patient is in remission. When using the method described herein for personalized medicine, it is appreciated that the more samples obtained at different time point, the more reliable the prediction for relapse would be.

In certain specific embodiments, if no change (or at least a statistical change) is observed in the rate of change of miR146a expression value and/or miR-146a regulated genes expression value compared to a respective predetermined standard rate of change, an additional sample from the same patient may be obtained at a later time point. Responsiveness, remission or relapse may be assessed based on the information obtained from the two measurements.

As shown in Example 3 provided herein below, a down regulation by at least 1.5 folds was observed in miR-146a regulated genes expression value during relapse compared to the same value when the patient was in remission. Thus, a decrease of at least 1.5 in the expression of miR-146a regulated genes is indicative for the patient to be considered in a relapse. At times, a decrease of at least 2, at least 3, at least 4, at least 5, at least 6, at least 7, at least 8 is sufficient to determine relapse of a patient.

The methods of the invention described herein, relate to interferon treatment, specifically, to assessing the responsiveness to interferon treatment. As used herein the term “interferon” or “IFN” which is interchangeably used herein, refers to a synthetic, recombinant or purified interferon, and encompasses interferon type I that binds to the cell surface receptor complex IFN-a receptor (IFNAR) consisting of IFNAR1 and IFNAR2 chains; interferon type II that binds to the IFNGR receptor; and interferon type III, that binds to a receptor complex consisting of IL10R2 (also called CRF2-4) and IFNLR1 (also called CRF2-12).

Interferon type I in human includes interferon alpha 1 (GenBank Accession No. NM_{—}024013 and NP_{—}076918; SEQ ID NOs: 7 and 8 respectively), interferon alpha 2 (GenBank Accession No. NM_{—}000605 and NP_{—}000596; SEQ ID NO: 9 and 10, respectively), Interferon alpha-4 (GenBank Accession No. NM_{—}021068 and NP_{—}066546; SEQ ID NO: 11 and 12, respectively), Interferon alpha-5 (GenBank Accession No. NM_{—}002169 and NP_{—}002160; SEQ ID NO: 13 and 14, respectively), Interferon alpha-6 (GenBank Accession No. NM_{—}021002 and NP_{—}066282; SEQ ID NO: 15 and 16, respectively), Interferon alpha-7 (GenBank Accession No. NM_{—}021057 and NP_{—}066401; SEQ ID NO: 17 and 18, respectively), Interferon alpha-8 (GenBank Accession No. NM_{—}002170 and NP_{—}002161; SEQ ID NO: 19 and 20, respectively), Interferon alpha-10 (GenBank Accession No. NM_{—}002171 and NP_{—}002162; SEQ ID NO: 21 and 22, respectively), Interferon alpha-1/13 (GenBank Accession No. NM_{—}006900 and NP_{—}008831; SEQ ID NO: 23 and 24, respectively), Interferon alpha-14 (GenBank Accession No. NM_{—}002172 and NP_{—}002163; SEQ ID NO: 25 and 26, respectively), Interferon alpha-16 (GenBank Accession No. NM_{—}002173 and NP_{—}002164; SEQ ID NO: 27 and 28, respectively), Interferon alpha-17 (GenBank Accession No. NM_{—}021268 and NP_{—}067091; SEQ ID NO: 29 and 30, respectively) and Interferon alpha-21 (GenBank Accession No. NM_{—}002175 and NP_{—}002166; SEQ ID NO: 31 and 32, respectively), Interferon, beta 1 (GenBank Accession No. NM_{—}002176 and NP_{—}002167; SEQ ID NO: 33 and 34, respectively), and Interferon omega-1 (GenBank Accession No. NM_{—}002177 and NP_{—}002168; SEQ ID NOs: 35 and 36 respectively)].

Interferon type II in humans is Interferon-gamma (GenBank Accession No. NM_{—}000619 and NP_{—}000610; SEQ ID NOs: 37 and 38 respectively).

As used herein the phrase “interferon treatment” refers to administration of interferon into a subject in need thereof. It should be noted that administration of interferon may comprise a single or multiple dosages, as well as a continuous administration, depending on the pathology to be treated and a clinical assessment of the subject receiving the treatment.

Various modes of interferon administration are known in the art. These include, but are not limited to, injection (e.g., using a subcutaneous, intramuscular, intravenous, or intradermal injection), intranasal administration and oral administration.

According to some embodiments of the invention, interferon treatment is provided to the subject in doses matching his weight, at a frequency of once a week, for a period of up to 48 weeks.

Non-limiting examples of interferon treatment and representative diseases includes the following interferon beta-1a (multiple sclerosis), interferon beta-1b (multiple sclerosis), recombinant IFN-a2b (various cancers).

As appreciated in the art, interferon alfa-2a treatment is known as Roferon. Interferon alpha 2b treatment is by Intron A or Reliferon or Uniferon. Interferon beta-1a is sold under the trade names Avonex and Rebif. CinnaGen is a biosimilar compound. Interferon beta-1b is sold under trade names Betaferon, Betaseron, Extavia and ZIFERON.

Interferon treatment may comprise PEGylated interferon i.e., conjugated to a polyethylene glycol (PEG) polymer. For example, PEGylated interferon alpha 2a is sold under the trade name Pegasys. PEGylated interferon alpha 2a in Egypt is sold under the trade name Reiferon Retard. PEGylated interferon alpha 2b is sold under the trade name PegIntron.

The interferon treatment can also comprise a combination of interferon and ribavirin. For example, PEGylated interferon alpha 2b plus ribavirin is sold under the trade name Pegetron.

The invention shows that the expression levels of miR-146a may be used as a prognostic tool distinguishing between interferon responders and non-responders and between subjects in relapse and subjects in remission.

Still further, as shown by Example 1, a group of genes regulated by miR-146a were shown as discriminating between populations of responders and non-responders, and in certain embodiments, between population of subjects in remission and subjects in relapse. In yet another embodiment, the miR-146a regulated genes may be selected from a group consisting of IFI44L, MX2, RSAD2, IFIT5, IFITM1, IFITM3, IRF7, ISG15, IF127, TRAF6, IF144, IFIT3, OASL, TRIM22, IFIT1, IRAK1 and IRAK2. Sequence information regarding these genes (i.e., RNA transcripts and polypeptide sequences) can be found in Table 1 in the Examples section which follows. In addition, probes and primers which can be used to detect transcripts of these genes are provided in herein after.

More specifically, it must be appreciated that the method of the invention may determine and use as a prognostic tool the expression value of any of the miR-146a regulated genes described herein below.

Interferon-induced protein 44-like (IFI44L) gene (GenBank Accession No. NM_{—}0068208; SEQ ID NO: 39) encodes the IFI44L protein (GenBank Accession No. NP_{—}006811; SEQ ID NO: 40) that belongs to the IF144 family of proteins is located in the cytoplasm and exhibits a low antiviral activity against hepatitis C. The expression of the protein is induced by type I interferon.

Myxovirus (influenza virus) resistance 2 (MX2) gene (GenBank Accession No. NM_{—}002463; SEQ ID NO: 41) encodes the MX2protein (GenBank Accession No. NP_{—}002454; SEQ ID NO: 42). MX2 is induced by interferon.

Radical S-adenosyl methionine domain containing 2 (RSAD2) gene (GenBank Accession No. NM_{—}080657; SEQ ID NO: 43) encodes the RSAD2 protein (GenBank Accession No. NP_{—}542388; SEQ ID NO: 44). RSAD2 is reported to be involved in antiviral defense. It was suggested to impair virus budding by disrupting lipid rafts at the plasma membrane, a feature which is essential for the budding process of many viruses. In addition, it was reported to act through binding with and inactivating FPPS, an enzyme involved in synthesis of cholesterol, farnesylated and geranylated proteins, ubiquinones dolichol and heme. Moreover, it is considered to play a major role in the cell antiviral state induced by type I and type II interferon. Finally, it was reported to display antiviral effect against HW-1 virus, hepatitis C virus, human cytomegalovirus, and aphaviruses, but not vesiculovirus.

Interferon-induced protein with tetratricopeptide repeats 5 (IFIT5) gene (GenBank Accession No. NM_{—}012420; SEQ ID NO: 45) encodes the FITS protein (GenBank Accession No. NP_{—}036552; SEQ ID NO: 46).

Interferon induced transmembrane protein 1 (IFITM1) gene (GenBank Accession No. NM_{—}003641; SEQ ID NO: 47) encodes the IFITM1 protein (GenBank Accession No. NP_{—}003632; SEQ ID NO: 48). IFITM1 is reported to be an IFN-induced antiviral protein that mediates cellular innate immunity to at least three major human pathogens, namely influenza A H1N1 virus, West Nile virus, and dengue virus by inhibiting the early step(s) of replication. It was also been reported to play a key role in the antiproliferative action of IFN-gamma either by inhibiting the ERK activation or by arresting cell growth in G1 phase. In addition, it was reported to implicate in the control of cell growth. Finally, it is regarded as a component of a multi-meric complex involved in the transduction of antiproliferative and homotypic adhesion signals.

Interferon induced transmembrane protein 3 (IFITM3) gene (GenBank Accession No. NM_{—}021034; SEQ ID NO: 49) encodes the IFITM3 protein (GenBank Accession No. NP_{—}066362; SEQ ID NO: 50). IFITM3 is reported to be IFN-induced antiviral protein that mediates cellular innate immunity to at least three major human pathogens, namely influenza A H1N1 virus, West Nile virus (WNV), and dengue virus (WNV), by inhibiting the early step(s) of replication.

Interferon regulatory factor 7 (IRF7) gene (GenBank Accession Nos. NM_{—}001572; SEQ ID NO: 51, NM_{—}004029; SEQ ID NO: 53) encodes the IRF7 protein (GenBank Accession Nos. NP_{—}001563; SEQ ID NO: 52, NP_{—}004020; SEQ ID NO: 54). IFR7 is reported to be a transcriptional activator. It binds to the interferon-stimulated response element (ISRE) in IFN promoters and in the Q promoter (Qp) of EBV nuclear antigen 1 (EBNA1). It is also reported to function as a molecular switch for antiviral activity. It is reported to be activated by phosphorylation in response to infection. The activation leads to nuclear retention, DNA binding, and depression of transactivation ability.

ISG15 ubiquitin-like modifier (ISG15) gene (GenBank Accession No. NM_{—}005101; SEQ ID NO: 55) encodes the ISG15 protein (GenBank Accession No. NM_{—}005101; SEQ ID NO: 56). ISG15 is reported to be an ubiquitin-like protein that is conjugated to intracellular target proteins after IFN-alpha or IFN-beta stimulation. Its enzymatic pathway is partially distinct from that of ubiquitin, differing in substrate specificity and interaction with ligating enzymes. ISG15 conjugation pathway uses a dedicated E1 enzyme, but seems to converge with the ubiquitin conjugation pathway at the level of a specific E2 enzyme. Targets include STAT1, SERPINA3G/SPI2A, JAK1, MAPK3/ERK1, PLCG1, EIF2AK2/PKR, MX1/MxA, and RIG-1. It undergoes deconjugation by USP18/UBP43. It shows specific chemotactic activity towards neutrophils and activates them to induce release of eosinophil chemotactic factors. It was suggested to serve as a trans-acting binding factor directing the association of ligated target proteins to intermediate filaments. Also it may also be involved in autocrine, paracrine and endocrine mechanisms, as in cell-to-cell signaling, possibly partly by inducing IFN-gamma secretion by monocytes and macrophages. It appeaser to display antiviral activity during viral infections In response to IFN-tau, ISG15 was reported to be secreted by the conceptus, may ligate to and regulate proteins involved in the release of prostaglandin F2-alpha (PGF), and thus prevent lysis of the corpus luteum and maintain the pregnancy.

Interferon alpha-inducible protein 27 (IF127) gene (GenBank Accession Nos. NM_{—}001130080 and NM_{—}005532; SEQ ID NOs:57, 59, respectively) encodes the IF127 protein (GenBank Accession Nos. NP_{—}001123552 and NP_{—}005523; SEQ ID NOs:58, 60, respectively). The IF127 protein was reported to promote cell death and mediate IFN-induced apoptosis characterized by a rapid and robust release of cytochrome C from the mitochondria and activation of BAX and caspases 2, 3, 6, 8 and 9.

TNF receptor-associated factor 6, E3 ubiquitin protein ligase (TRAF6) gene (GenBank Accession Nos. NM_{—}145803 and NM_{—}004620; SEQ ID NOs:61, 63, respectively) encodes the TRAF6 protein (GenBank Accession Nos. NP_{—}665802 and NP_{—}004611; SEQ ID NOs:62, 64, respectively). The TRAF6 protein is an E3 ubiquitin ligase that, together with UBE2N and UBE2V1, mediates the synthesis of ‘Lys-63’-linked-polyubiquitin chains conjugated to proteins, such as IKBKG, AKT1 and AKT2. It was also shown to mediate ubiquitination of free/unanchored polyubiquitin chain that leads to MAP3K7 activation. In addition, it was shown to lead to the activation of NF-kappa-B and JUN. Further it was suggested to be essential for the formation of functional osteoclasts and seems to also play a role in dendritic cells (DCs) maturation and/or activation. Further, it was shown to repress c-Myb-mediated transactivation, in B-lymphocytes. Finally, TRAF6 is considered as an adapter protein that seems to play a role in signal transduction initiated via TNF receptor, IL-1 receptor and IL-17 receptor.

Interferon-induced protein 44 (IF144) gene (GenBank Accession No. NM_{—}006417; SEQ ID NO: 65) encodes the IF144 protein (GenBank Accession No. NP_{—}006408; SEQ ID NO: 66), that was reported to aggregate to form microtubular structures.

Interferon-induced protein with tetratricopeptide repeats 3 (IFIT3) gene (GenBank Accession Nos. NM_{—}001031683; SEQ ID NO: 67, NM_{—}001549; SEQ ID NO: 69) encodes the FIT3 protein (GenBank Accession Nos. NP_{—}001026853; SEQ ID NO: 68, NP_{—}001540; SEQ ID NO: 70).

2 ‘-5’-oligoadenylate synthetase-like (OASL) gene (GenBank Accession Nos. NM_{—}003733; SEQ ID NO: 71, NM_{—}198213; SEQ ID NO: 73) encodes the OASL protein (GenBank Accession Nos. NP_{—}003724; SEQ ID NO: 72, NP_{—}937856; SEQ ID NO: 74).

Tripartite motif containing 22 (TRIM22) gene (GenBank Accession Nos. NM_{—}001199573; SEQ ID NO: 75, NM_{—}006074; SEQ ID NO: 77) encodes the TRIM22 protein (GenBank Accession Nos. NP_{—}001186502; SEQ ID NO: 76, NP_{—}006065; SEQ ID NO: 78). Trim22 is reported to be an interferon-induced antiviral protein involved in cell innate immunity, with the antiviral activity could in part be mediated by TRIM22-dependent ubiquitination of viral proteins. In addition, it is reported to play a role in restricting the replication of HIV-1, encephalomyocarditis virus (EMCV) and hepatitis B virus (HBV). It was acts as a transcriptional repressor of HBV core promoter. Finally it was suggested to have E3 ubiquitin-protein ligase activity.

Interferon-induced protein with tetratricopeptide repeats 1 (IFIT1) gene (GenBank Accession No. NM_{—}001548; SEQ ID NO: 79) encodes the IRF1 protein (GenBank Accession No. NP_{—}001539; SEQ ID NO: 80).

Interleukin-1 receptor-associated kinase 1 (IRAK1) gene (GenBank Accession Nos. NM_{—}001025242; SEQ ID NO: 81, NM_{—}001025243; SEQ ID NO: 83, NM_{—}001569; SEQ ID NO: 85) encodes the IRAK1 protein (GenBank Accession Nos. NP_{—}001020413; SEQ ID NO: 82, NP_{—}001020414; SEQ ID NO: 84, NP_{—}001560; SEQ ID NO: 86). The IRAK1 gene encodes the interleukin-1 receptor-associated kinase 1, one of two putative serine/threonine kinases that become associated with the interleukin-1 receptor (IL1R) upon stimulation.

Interleukin-1 receptor-associated kinase 2 (IRAK2) gene (GenBank Accession No. NM_{—}001570; SEQ ID NO: 87) encodes the IRAK2 protein (GenBank Accession No. NP NP_{—}001561; SEQ ID NO: 88). IRAK2 gene encodes the interleukin-1 receptor-associated kinase 2, one of two putative serine/threonine kinases that become associated with the interleukin-1 receptor (IL1R) upon stimulation. IRAK2 is reported to participate in the IL1-induced upregulation of NF-kappaB.

In accordance with the present invention, the level of expression of miR-146a and optionally of at least one of miR-146a regulated genes is determined in a biological sample of said subject to obtain an expression value.

According to some specific embodiments, the method of the invention involves the determination of the level of expression of miR-146a in a biological sample of the examined subject to obtain an expression value.

In yet further embodiments, the methods of the invention require determining the expression level of miR-146a and of at least one, at least two, at least three, at least four, at least five, at least six, at least seven, at least eight, at least nine, at least ten, at least eleven, at least twelve, at least thirteen, at least fourteen, at least fifteen, at least sixteen or at least seventeen of said miR-146a regulated genes as described by the invention in a biological test sample of a mammalian subject.

Other embodiments of the invention relate to the use of different combinations of miR-146a with different specific miR-146a regulated genes.

More specifically, the present invention partly relates to changes in the expression level of miR-146a regulated gens, however as may be appreciated, there may be variations in the changes observed in the expression levels of the miR-146a regulated genes as determined in the biological sample. Namely, the changes in the expression of the miR-146a regulated genes may not be in the same magnitude.

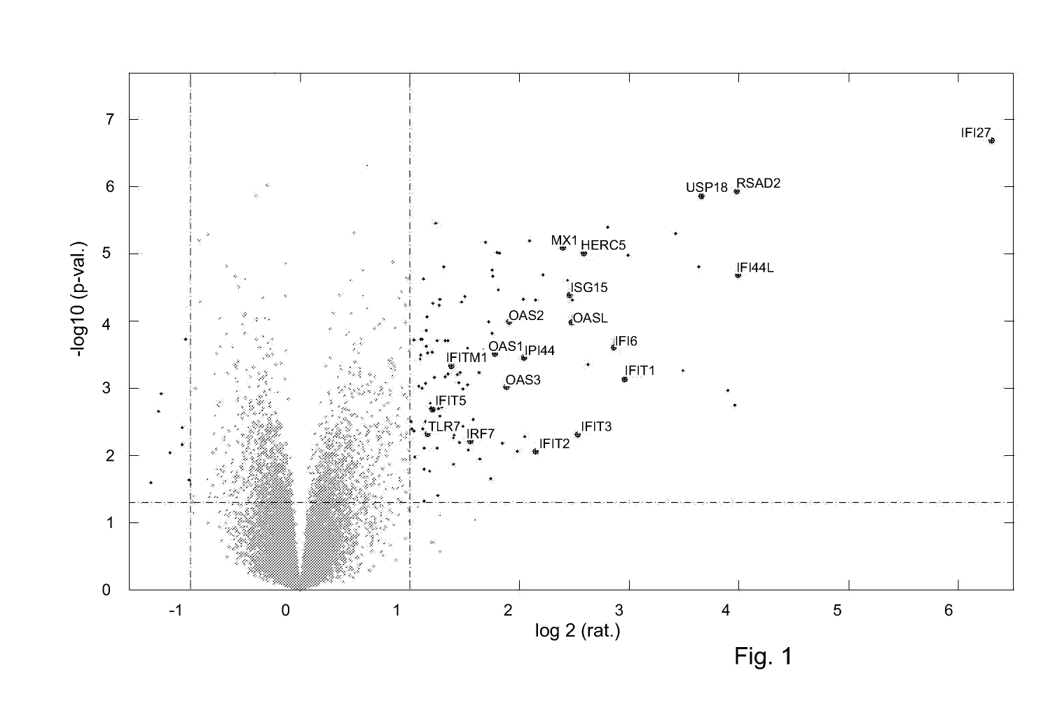

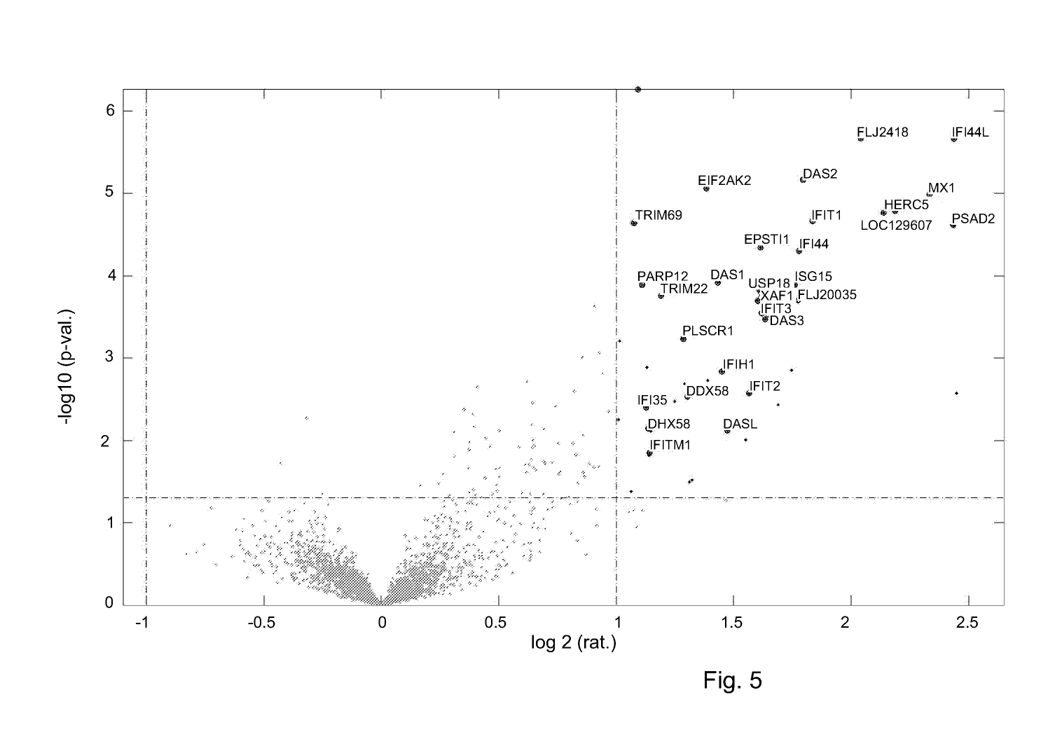

For example, as shown in FIG. 1 herein showing gens distribution in MS patients after interferon treatment, the changes observed in the expression value of IF127, RSAD2 and IFI44L after treatment in responders are the most significant. Further, as shown in FIG. 5 the most significant changes are observed in the expression values of IFI44L and RSAD2.

Thus, according to some specific embodiments, the level of expression of miR-146a, of IF127 gene and optionally of any one of the miR-146a regulated genes is determined in a biological sample of the tested subject to obtain an expression value. In some other specific embodiments, the level of expression of miR-146a and ofIFI27 gene is determined in a biological sample of said subject to obtain an expression value.

According to some other specific embodiments, the level of expression of miR-146a, of RSAD2 gene and optionally of any one of miR-146a regulated genes is determined in a biological sample of said subject to obtain an expression value. In some other specific embodiments, the level of expression of miR-146a and ofRSAD2 gene is determined in a biological sample of said subject to obtain an expression value.

According to some other embodiments, the level of expression of miR-146a, of RSAD2, of IF127 and optionally of any one of miR-146a regulated genes is determined in a biological sample of said subject to obtain an expression value. In some further embodiments, the level of expression of miR-146a and of at least two genes, namely, RSAD2 and IF127 is determined in a biological sample of said subject to obtain an expression value.

According to some other embodiments, the level of expression of miR-146a, of IFI44L gene and optionally of any one of miR-146a regulated is determined in a biological sample of said subject to obtain an expression value. In some further embodiments, the level of expression of miR-146a and of IFI44L gene is determined in a biological sample of said subject to obtain an expression value.

According to some other embodiments, the level of expression of miR-146a and of at least two, specifically, RSAD2 and IFI44L, and optionally of any one of miR-146a regulated gene is determined in a biological sample of said subject to obtain an expression value. In some further embodiments, the level of expression of miR-146a and of at least two genes, specifically, RSAD2 and IFI44L genes is determined in a biological sample of said subject to obtain an expression value.

According to some other embodiments, the level of expression of miR-146a and of at least seven genes, specifically, of RSAD2, IF127, IFI44L, IFIT1, IF144, ISG15, IFIT3 and OASL and optionally of any other miR-146a regulated genes is determined in a biological sample of said subject to obtain an expression value. In some further embodiments, the level of expression of miR-146a and ofRSAD2, IF127, IFI44L, IFIT1, IF144, ISG15, IFIT3 and OASL gene is determined in a biological sample of said subject to obtain an expression value.

According to some specific embodiments, for determining responsiveness to interferon treatment in MS patients, the level of expression of miR-146a and of at least eleven regulated genes, specifically, RSAD2, IF127, IFI44L, IFIT1, ISG15, IFIT3, OASL, IF144, IFITM1, IRF7 and IFIT5, and optionally, any further miR-146a regulated genes is determined in a biological sample of said subject to obtain an expression value. In some further embodiments, for determining responsiveness to interferon treatment in MS patients, the level of expression of miR-146a and of RSAD2, IF127, IFI44L, IFIT′, IF144, ISG15, IFIT3 and OASL genes is determined in a biological sample of said subject to obtain an expression value.

According to some further specific embodiments, for determining responsiveness to interferon treatment in MS patients, the level of expression of miR-146a and of at least seven miR-146a regulated genes, namely, IF127, RSAD2, IFI44L, IFIT1, ISG15, IFIT3 and OASL, and optionally of further miR-146a regulated genes is determined in a biological sample of said subject to obtain an expression value. In some further embodiments, for determining responsiveness to interferon treatment in MS patients, the level of expression of miR-146a and ofRSAD2, IF127, IFI44L, IFIT1, ISG15, IFIT3 and OASL gene is determined in a biological sample of said subject to obtain an expression value.

According to some specific embodiments, for determining responsiveness to interferon treatment in HCV patients, the level of expression of miR-146a and of at least nine miR-146a regulated genes, specifically, IFI44L, RSAD2, IFIT1, IF144, ISG15, IFIT3, OASL, TRIM22, IFITM1, and optionally, any other miR-146a regulated gene is determined in a biological sample of said subject to obtain an expression value. In some further embodiments, for determining responsiveness to interferon treatment in HCV patients, the level of expression of miR-146a and ofIFI44L, RSAD2, IFIT1, IF144, ISG15, IFIT3, OASL, TRIM22 and IFITM1 genes is determined in a biological sample of said subject to obtain an expression value.

In some further embodiments, for determining responsiveness to interferon treatment in HCV patients, the level of expression of miR-146a and optionally of at least six miR-146a regulated genes, for example, IFI44L, RSAD2, IFIT1, IF144, ISG15, IFIT3 and optionally any other miR-146a regulated gene is determined in a biological sample of said subject to obtain an expression value. In some further specific embodiments, for determining responsiveness to interferon treatment in HCV patients, the level of expression of miR-146a and ofIFI44L, RSAD2, IFIT1, IF144, ISG15 and IFIT3 genes is determined in a biological sample of said subject to obtain an expression value.

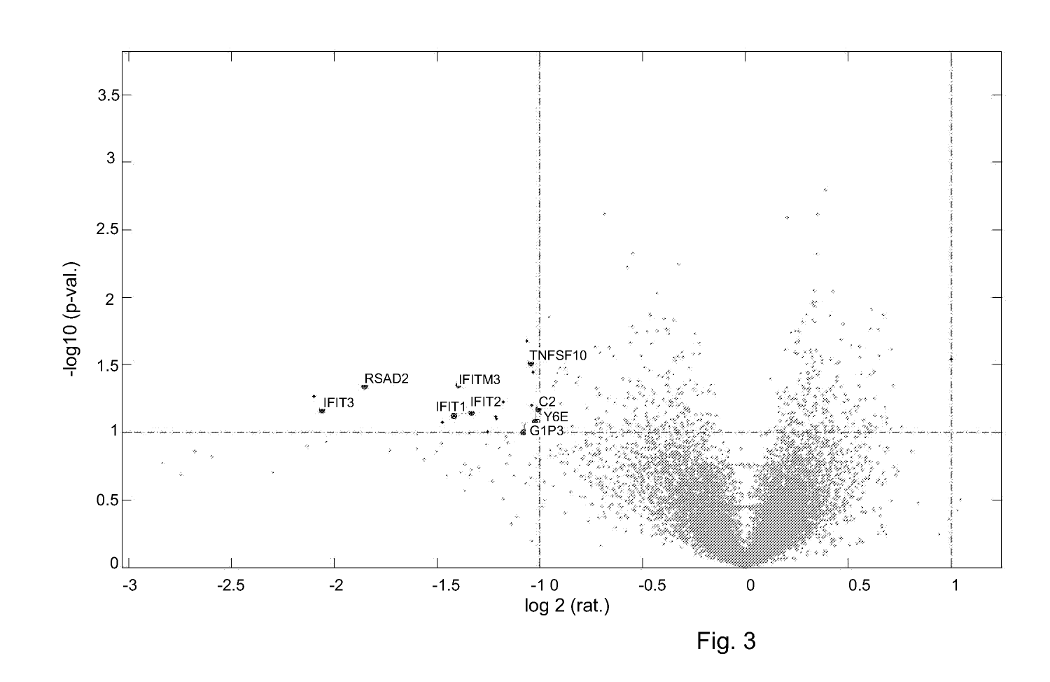

Further, as shown in FIG. 3 herein showing gens distribution in MS patients when experiencing relapse vs. when stable, the expression value of IFIT3 and RSAD2 are significantly down regulated during relapse.

Thus, according to some specific embodiments, to determine relapse in MS patients, the level of expression of miR-146a and of at least two genes, specifically, IFIT3, RSAD2 and optionally, any other miR-146a regulated gene is determined in a biological sample of said subject to obtain an expression value. In some other specific embodiments, the level of expression of miR-146a and of IFIT3 and RSAD2 genes is determined in a biological sample of said subject to obtain an expression value.

According to some other specific embodiments, the level of expression of miR-146a and at least four miR-146a regulated genes, specifically, IFIT3, RSAD2, IFITM3 and IFIT1, and optionally of any other miR-146a regulated gene is determined in a biological sample of said subject to obtain an expression value. In some other specific embodiments, the level of expression of miR-146a and of IFIT3, RSAD2, IFITM3 and IFIT1 genes is determined in a biological sample of said subject to obtain an expression value. In yet another embodiment, in addition to the combinations described above, the method of the invention may optionally further comprise the step of determining the level of expression of any other miR-146a regulated gene, for example, at least one of CCL2, SERPING1, LAMP3, CFB, G1P3, TNFSF10, LY6E. In more specific embodiments, the level of expression of miR-146a and of at least one of G1P3, TNFSF10 and LY6E may be determined.

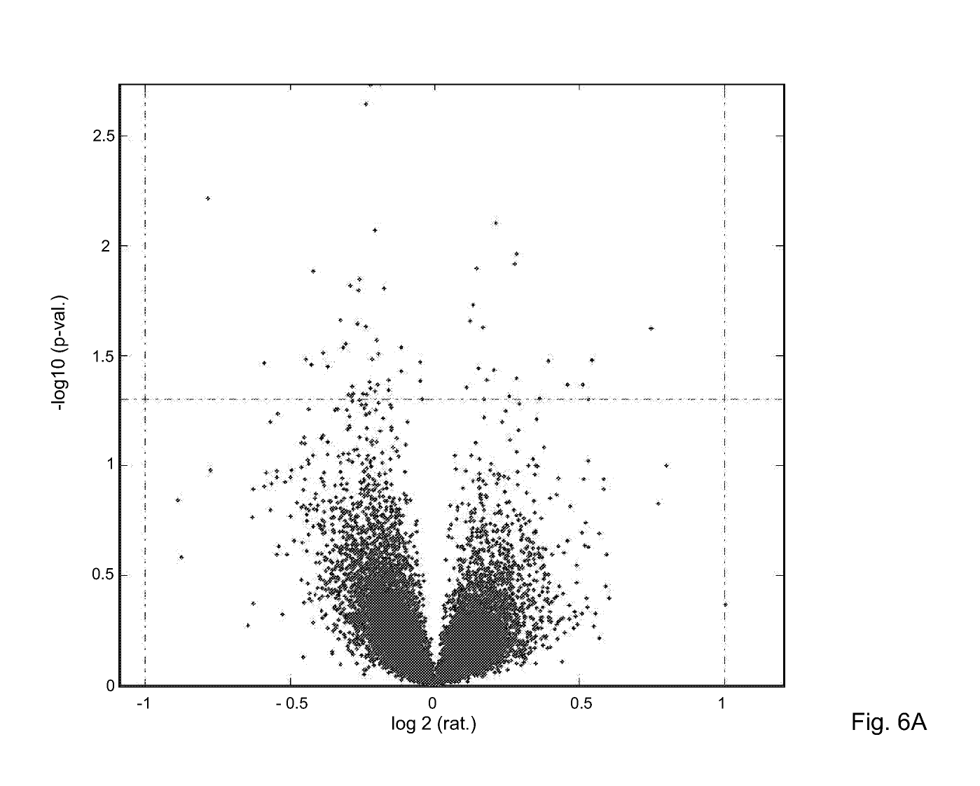

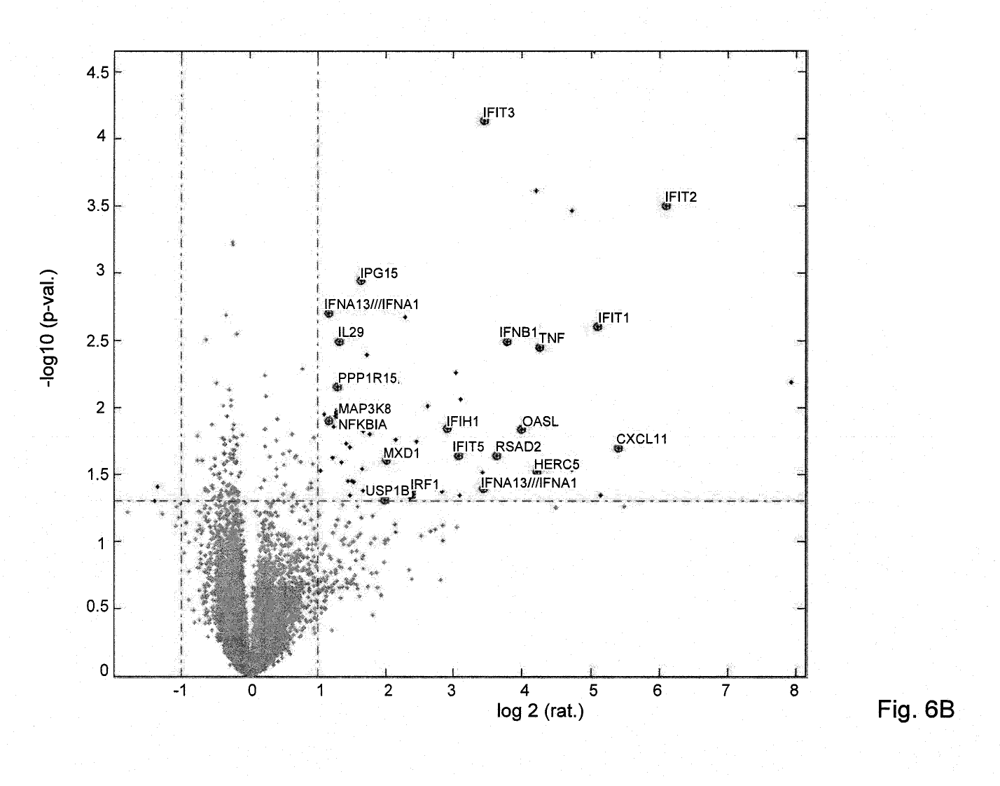

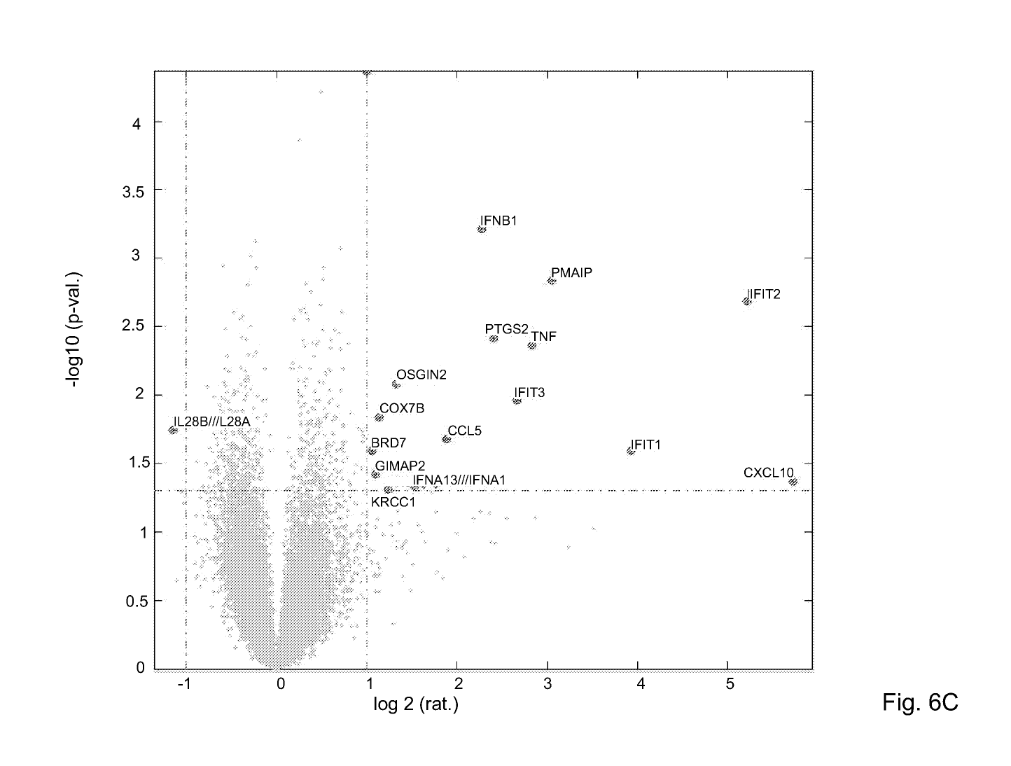

Still further, as shown in FIG. 6 herein showing gene distribution in H1N1 and H5N1 infected cells the changes observed in the expression value of IFIT2, IFIT1 and IFIT3 are significantly up regaled 6 hours after infection.

According to some other specific embodiments, the level of expression of miR-146a and of at least three miR-146a regulated genes, specifically IFIT2, IFIT1 and IFIT3 and optionally of any other miR-146a regulated gene is determined in a biological sample of said subject to obtain an expression value. In some other specific embodiments, the level of expression of miR-146a and ofIFIT2, IFIT1 and IFIT3 gene is determined in a biological sample of said subject to obtain an expression value.

According to some further specific embodiments, the level of expression of miR-146a and of at least six miR-146a regulated genes, specifically, IFIT2, IFIT1, IFIT3, OASL, RSDA2 and IFIT5, and optionally of any other miR-146a regulated gene is determined in a biological sample of said subject to obtain an expression value. In some other specific embodiments, the level of expression of miR-146a and ofIFIT2, IFIT1, IFIT3, OASL, RSDA2 and IFIT5 gene is determined in a biological sample of said subject to obtain an expression value.

According to some specific embodiments, to determine if a subject infected with a viral disease for example influenza will respond to interferon treatment, the level of expression of miR-146a and of at least six miR-146a regulated genes, specifically, IFIT2, IFIT1, IFIT3, OASL, RSDA2 and IFIT5, and optionally any further miR-146a regulated gene is determined in a biological sample of said subject to obtain an expression value. In some further specific embodiments, to determine if a subject infected with a viral disease for example influenza will respond to interferon treatment, the level of expression of miR-146a and of IFIT2, IFIT1, IFIT3, OASL, RSDA2 and IFIT5 gene is determined in a biological sample of said subject to obtain an expression value.

According to some further embodiments, the level of expression of miR-146a and of at least seventeen miR-146a regulated genes, specifically, IFI44L, MX2, RSAD2, IFIT5, IFITM1, IFITM3, IRF7, ISG15, IF127, TRAF6, IF144, IFIT3, OASL, TRIM22, IFIT1, IRAK1 and IRAK2, and optionally any further miR-146a regulated gene, is determined in a biological sample of said subject to obtain an expression value. In some other specific embodiments, the level of expression of miR-146a and of IFI44L, MX2, RSAD2, IFIT5, IFITM1, IFITM3, IRF7, ISG15, IF127, TRAF6, IF144, IFIT3, OASL, TRIM22, IFIT1, IRAK1 and IRAK2 genes is determined in a biological sample of said subject to obtain an expression value. Still further, according to another specific embodiment, the method of the invention comprises the step of determining the level of expression of IFI44L, MX2, RSAD2, IFIT5, IFITM1, IFITM3, IRF7, ISG15, IF127, TRAF6, IF144, IFIT3, OASL, TRIM22, IFIT1, IRAK1, and IRAK2 in a sample of the tested subject.

In yet some specific embodiments, the method of the invention involves determining the level of expression of any one of IFI44L, MX2, RSAD2, IFIT5, IFITM1, IFITM3, IRF7, ISG15, IF127, TRAF6, IF144, IFIT3, OASL, TRIM22, IFIT1, IRAK1, IRAK2 and any combination thereof and optionally, any combinations thereof with any other miR-146a regulated genes, in a sample obtained from the tested subject. In one specific embodiment, such other miR-146a regulated genes may include at least one of CCL2, SERPING1, LAMP3, CFB, G1P3, TNFSF10, LY6E, specifically, G1P3, TNFSF10, LY6E. It should be noted that any combination of these genes is encompassed by the invention provided that said combination is not any one of OAS3, IF16, ISG15, OAS2, IFIT1, KIR3DL3, KIR3DL2, KIR3DL1, KIR2DL1, KIR2DL2, KIR2DL3, KLRG1, KIR3DS1, CD160, HLA-A, HLA-B, HLA-C, HLA-F, HLA-G and IF127 or OAS3, IF16, ISG15, OAS2 and IFIT1. In yet another embodiment, the method of the invention encompasses the option of determining the level of expression of at least one of IFI44L, MX2, RSAD2, IFIT5, IFITM1, IFITM3, IRF7, TRAF6, IF144, IFIT3, OASL, TRIM22, IRAK1, and IRAK2.

According to specific embodiments, determining the level of expression of miR-146a and optionally of at least one of miR-146a regulated genes in a biological sample of the examined subject may be performed by the step of contacting detecting molecules specific for miR-146a and optionally for at least one of miR-146a regulated genes with a biological sample of said subject, or with any nucleic acid or protein product obtained therefrom.

As indicated above, the first step of the diagnostic method of the invention may involve contacting the sample or any aliquot thereof with detecting molecules specific for miR-146a and optionally of at least one of miR-146a regulated genes.

The term “contacting” means to bring, put, incubate or mix together. As such, a first item is contacted with a second item when the two items are brought or put together, e.g., by touching them to each other or combining them. In the context of the present invention, the term “contacting” includes all measures or steps which allow interaction between the at least one of the detection molecules for miR-146a and at least one of miR-146a regulated genes and optionally one suitable control reference gene or miRNA and the nucleic acid or amino acid molecules of the tested sample. The contacting is performed in a manner so that the at least one of detecting molecule of miR-146a and miR-146a regulated genes and at least one suitable control reference gene or miRNA can interact with or bind to the nucleic acid molecules or alternatively, a protein product of the at least one miR-146a regulated genes, in the tested sample. The binding will preferably be non-covalent, reversible binding, e.g., binding via salt bridges, hydrogen bonds, hydrophobic interactions or a combination thereof.

In certain embodiments, the detection step further involves detecting a signal from the detecting molecules that correlates with the expression level of said miR-146a or miR-146a regulated genes or product by a suitable means thereof in the sample from the subject. According to some embodiments, the signal detected from the sample by any one of the experimental methods detailed herein below reflects the expression level of said miR-146a or miR-146a regulated genes or product thereof. Such signal-to-expression level data may be calculated and derived from a calibration curve.

Thus, in certain embodiments, the method of the invention may optionally further involve the use of a calibration curve created by detecting a signal for each one of increasing pre-determined concentrations of said miR-146a or miR-146a regulated genes or product. Obtaining such a calibration curve may be indicative to evaluate the range at which the expression levels correlate linearly with the concentrations of said miR-146a or miR-146a regulated genes or product. It should be noted in this connection that at times when no change in expression level of miR-146a or miR-146a regulated genes or product is observed, the calibration curve should be evaluated in order to rule out the possibility that the measured expression level is not exhibiting a saturation type curve, namely a range at which increasing concentrations exhibit the same signal.

It must be appreciated that in certain embodiments such calibration curve as described above may by also part or component in any of the kits provided by the invention herein after.

In other embodiments of the invention, the detecting molecules used for determining the expression levels of the biomarkers of the invention, may be either isolated detecting nucleic acid molecules or isolated detecting amino acid molecules. It should be noted that the invention further encompasses any combination of nucleic and amino acids for use as detecting molecules for the method of the invention. As noted above, in the first step of the method of the invention, the sample or any nucleic acid or protein product derived therefrom is contacted with the detecting molecules of the invention.

In more specific embodiments, for determining the expression level of the biomarkers of the invention, nucleic acid detecting molecule may be used. More specifically, such nucleic acid detecting molecules may comprise isolated oligonucleotides, each oligonucleotide specifically hybridizes to a nucleic acid sequence of miR-146a or of at least one of miR-146a regulated genes. In an optional embodiment, were the expression level of the biomarkers of the invention are normalized, the method of the invention may use nucleic acid detecting molecules specific for a control miRNA or control reference gene.

According to more specific embodiment, the nucleic acid detecting molecules used by the method of the invention may be at least one of a pair of primers or nucleotide probes.

As used herein, “nucleic acids” or “nucleic acid sequence” are interchangeable with the term “polynucleotide(s)” and it generally refers to any polyribonucleotide or poly-deoxyribonucleotide, which may be unmodified RNA or DNA or modified RNA or DNA or any combination thereof. “Nucleic acids” include, without limitation, single- and double-stranded nucleic acids. As used herein, the term “nucleic acid(s)” also includes DNAs or RNAs as described above that contain one or more modified bases. Thus, DNAs or RNAs with backbones modified for stability or for other reasons are “nucleic acids”. The term “nucleic acids” as it is used herein embraces such chemically, enzymatically or metabolically modified forms of nucleic acids, as well as the chemical forms of DNA and RNA characteristic of viruses and cells, including for example, simple and complex cells. A “nucleic acid” or “nucleic acid sequence” may also include regions of single- or double-stranded RNA or DNA or any combinations.

As used herein, the term “oligonucleotide” is defined as a molecule comprised of two or more deoxyribonucleotides and/or ribonucleotides, and preferably more than three. Its exact size will depend upon many factors which in turn, depend upon the ultimate function and use of the oligonucleotide. The oligonucleotides may be from about 3 to about 1,000 nucleotides long. Although oligonucleotides of 5 to 100 nucleotides are useful in the invention, preferred oligonucleotides range from about 5 to about 15 bases in length, from about 5 to about 20 bases in length, from about 5 to about 25 bases in length, from about 5 to about 30 bases in length, from about 5 to about 40 bases in length or from about 5 to about 50 bases in length. More specifically, the detecting oligonucleotides molecule used by the composition of the invention may comprise any one of 5, 6, 7, 8, 9, 10, 11, 12, 13, 14, 15, 16, 17, 18, 19, 20, 21, 22, 23, 24, 25, 26, 27, 28, 29, 30, 35, 40, 45, 50 bases in length. It should be further noted that the term “oligonucleotide” refers to a single stranded or double stranded oligomer or polymer of ribonucleic acid (RNA) or deoxyribonucleic acid (DNA) or mimetics thereof. This term includes oligonucleotides composed of naturally-occurring bases, sugars and covalent internucleoside linkages (e.g., backbone) as well as oligonucleotides having non-naturally-occurring portions which function similarly.

As indicated throughout, in certain embodiments when the detecting molecules used are nucleic acid based molecules, specifically, oligonucleotides. It should be noted that the oligonucleotides used in here specifically hybridize to nucleic acid sequences of miR-146a. Optionally, where also the expression of at least one of miR-146a regulated genes is being examined, the method of the invention may use as detecting molecules oligonucleotides that specifically hybridize to a nucleic acid sequence of said at least one miR-146a regulated genes. As used herein, the term “hybridize” refers to a process where two complementary nucleic acid strands anneal to each other under appropriately stringent conditions. Hybridizations are typically and preferably conducted with probe-length nucleic acid molecules, for example, 5-100 nucleotides in length, 5-50, 5-40, 5-30 or 5-20.

As used herein “selective or specific hybridization” in the context of this invention refers to a hybridization which occurs between a polynucleotide encompassed by the invention as detecting molecules, and miR-146a and/or at least one of miR-146a regulated gene and/or any control reference gene or miRNA, wherein the hybridization is such that the polynucleotide binds to miR-146a or to at least one of miR-146a regulated gene or any control reference gene or miRNA preferentially to any other RNA in the tested sample. In a specific embodiment a polynucleotide which “selectively hybridizes” is one which hybridizes with a selectivity of greater than 60 percent, greater than 70 percent, greater than 80 percent, greater than 90 percent and most preferably on 100 percent (i.e. cross hybridization with other RNA species preferably occurs at less than 40 percent, less than 30 percent, less than 20 percent, less than 10 percent). As would be understood to a person skilled in the art, a detecting polynucleotide which “selectively hybridizes” to miR-146a and at least one of miR-146a regulated genes or any control reference gene or miRNA can be designed taking into account the length and composition.

The terms, “specifically hybridizes”, “specific hybridization” refers to hybridization which occurs when two nucleic acid sequences are substantially complementary (at least about 60 percent complementary over a stretch of at least 5 to 25 nucleotides, preferably at least about 70 percent, 75 percent, 80 percent or 85 percent complementary, more preferably at least about 90 percent complementary, and most preferably, about 95 percent complementary).

The measuring of the expression of any one of miR-146a and at least one of miR-146a regulated genes and any control reference gene or miRNA and combination thereof can be done by using those polynucleotides as detecting molecules, which are specific and/or selective for miR-146a and/or at least one of miR-146a regulated genes or any control reference gene or miRNA to quantitate the expression of said miR-146a and at least one of miR-146a regulated genes or any control reference gene or miRNA. In a specific embodiment of the invention, the polynucleotides which are specific and/or selective for said miR-146a and at least one of miR-146a regulated genes or any control reference gene or miRNA may be probes or a pair of primers. It should be further appreciated that the methods, as well as the compositions and kits of the invention may comprise, as an oligonucleotide-based detection molecule, both primers and probes.

The term, “primer”, as used herein refers to an oligonucleotide, whether occurring naturally as in a purified restriction digest, or produced synthetically, which is capable of acting as a point of initiation of synthesis when placed under conditions in which synthesis of a primer extension product, which is complementary to a nucleic acid strand, is induced, i.e., in the presence of nucleotides and an inducing agent such as a DNA polymerase and at a suitable temperature and pH. The primer may be single-stranded or double-stranded and must be sufficiently long to prime the synthesis of the desired extension product in the presence of the inducing agent. The exact length of the primer will depend upon many factors, including temperature, source of primer and the method used. For example, for diagnostic applications, depending on the complexity of the target sequence, the oligonucleotide primer typically contains 10-30 or more nucleotides, although it may contain fewer nucleotides. More specifically, the primer used by the methods, as well as the compositions and kits of the invention may comprise 10, 11, 12, 13, 14, 15, 16, 17, 18, 19, 20, 21, 22, 23, 24, 25, 26, 27, 28, 29 or 30 nucleotides or more. In certain embodiments, such primers may comprise 30, 40, 50, 60, 70, 80, 90, 100 nucleotides or more. In specific embodiments, the primers used by the method of the invention may have a stem and loop structure. The factors involved in determining the appropriate length of primer are known to one of ordinary skill in the art and information regarding them is readily available.

As used herein, the term “probe” means oligonucleotides and analogs thereof and refers to a range of chemical species that recognize polynucleotide target sequences through hydrogen bonding interactions with the nucleotide bases of the target sequences. The probe or the target sequences may be single- or double-stranded RNA or single- or double-stranded DNA or a combination of DNA and RNA bases. A probe is at least 5 or preferably, 8 nucleotides in length and less than the length of a complete miRNA. A probe may be 5, 6, 7, 8, 9, 10, 11, 12, 13, 14, 15, 16, 17, 18, 19, 20, 21, 22, 23, 24, 25, 26, 27, 28, 29 and up to 30 nucleotides in length as long as it is less than the full length of the target miRNA or any gene encoding said miRNA. Probes can include oligonucleotides modified so as to have a tag which is detectable by fluorescence, chemiluminescence and the like. The probe can also be modified so as to have both a detectable tag and a quencher molecule, for example TaqMan(R) and Molecular Beacon(R) probes, that will be described in detail below.

The oligonucleotides and analogs thereof may be RNA or DNA, or analogs of RNA or DNA, commonly referred to as antisense oligomers or antisense oligonucleotides. Such RNA or DNA analogs comprise, but are not limited to, 2-′0-alkyl sugar modifications, methylphosphonate, phosphorothiate, phosphorodithioate, formacetal, 3-thioformacetal, sulfone, sulfamate, and nitroxide backbone modifications, and analogs, for example, LNA analogs, wherein the base moieties have been modified. In addition, analogs of oligomers may be polymers in which the sugar moiety has been modified or replaced by another suitable moiety, resulting in polymers which include, but are not limited to, morpholino analogs and peptide nucleic acid (PNA) analogs. Probes may also be mixtures of any of the oligonucleotide analog types together or in combination with native DNA or RNA. At the same time, the oligonucleotides and analogs thereof may be used alone or in combination with one or more additional oligonucleotides or analogs thereof.

In some specific embodiments, an anti-miRNA comprises the complement of a sequence of a miRNA referred to in SEQ ID NOs: 1 and 2. Preferred molecules are those that are able to hybridize under stringent conditions to the complement of a cDNA encoding a mature miR-146a, for example SEQ ID NO: 1. Particular antisense sequence for miR-146a is provided in SEQ ID NO: 89.

In yet more specific embodiment, detecting molecules specific for miR-146a may be oligonucleotides that specifically recognize and hybridize the miR-146a nucleic acid sequence. Specific, particular and non limiting example for such detecting molecule for miR-146a may be a probe sequence of miR-146a as denoted by SEQ ID NO. 92. In yet another specific, particular and non limiting examples for such detecting molecules for miR-146a may be primer sequence for real-time PCR such as the forward primer sequence as denoted by SEQ ID NO:93 and the reverse primer sequence as denoted by SEQ ID NO:94.

In yet another embodiment, the detecting molecules specific for miR-146a primary transcript may include the forward primer as denoted by any one of SEQ ID NO:95, SEQ ID NO:96, SEQ ID NO:97 or SEQ ID NO:98 and the reverse primer sequences as denoted by any one of SEQ ID NO:99, SEQ ID NO:100, SEQ ID NO:101 or SEQ ID NO:102, respectively.

According to certain embodiments, the methods of the invention, as well as the compositions and kits described herein after, may use detecting molecules specific for any of the miR-146a regulated genes. Non limiting examples relate to the use of specific probes. More specifically, probes sets suitable for determining the expression of miR-146a regulated genes may include IFI44L—Probe Set 204439 as denoted by SEQ ID NO:103. For MX2—Probe Set 204994 as denoted by SEQ ID NO:104, for RSAD2—Probe Set 213797_as denoted by SEQ ID NO:105. For IFIT5—Probe Set 203595_s_as denoted by SEQ ID NO:106, may be used. For IFITM1—Probe Set 201601_x_as denoted by SEQ ID NO:107, for IFITM1—Probe Set 214022_s_as denoted by SEQ ID NO:108, for IFITM3—Probe Set 212203_x_as denoted by SEQ ID NO:109, for IRF7—Probe Set 208436_s_as denoted by SEQ ID NO:110, for ISG15—Probe Set 205483_s_as denoted by SEQ ID NO:111, for IF127—Probe Set 202411_as denoted by SEQ ID NO:112, for TRAF6—Probe Set 205558_as denoted by SEQ ID NO:113. For IF144—Probe Set 214453_s_as denoted by SEQ ID NO:114, for IFIT3—Probe Set 204747_as denoted by SEQ ID NO:115, for OASL—Probe Set 205660_as denoted by SEQ ID NO:116, for OASL—Probe Set 210797_s_as denoted by SEQ ID NO:117, for TRIM22—Probe Set 213293_s_as denoted by SEQ ID NO:118 may be used. For IFIT1—Probe Set 203153_as denoted by SEQ ID NO:119 may be used. For IRAK1—Probe Set 201587_s_as denoted by SEQ ID NO:120, for IRAK1—Probe Set 1555784_s_as denoted by SEQ ID NO:121, for IRAK2—Probe Set 1553740_a_as denoted by SEQ ID NO:90 and for IRAK2—Probe Set 231779_as denoted by SEQ ID NO:91, may be used.

It should be appreciated that the detecting molecules described herein for miR-146a and the regulated genes are only non limiting examples. These examples may be also applicable for other aspects of the invention, namely, the compositions and kits described herein after.

Thus, according to one embodiment, such oligonucleotides are any one of a pair of primers or nucleotide probes, and wherein the level of expression of at least one of the miR-146a and at least one of miR-146a regulated genes is determined using a nucleic acid amplification assay selected from the group consisting of: a Real-Time PCR, micro array, PCR, in situ hybridization and comparative genomic hybridization.

The term “amplification assay”, with respect to nucleic acid sequences, refers to methods that increase the representation of a population of nucleic acid sequences in a sample. Nucleic acid amplification methods, such as PCR, isothermal methods, rolling circle methods, etc., are well known to the skilled artisan. More specifically, as used herein, the term “amplified”, when applied to a nucleic acid sequence, refers to a process whereby one or more copies of a particular nucleic acid sequence is generated from a template nucleic acid, preferably by the method of polymerase chain reaction.

“Polymerase chain reaction” or “PCR” refers to an in vitro method for amplifying a specific nucleic acid template sequence. The PCR reaction involves a repetitive series of temperature cycles and is typically performed in a volume of 50-100 microliter. The reaction mix comprises dNTPs (each of the four deoxynucleotides dATP, dCTP, dGTP, and dTTP), primers, buffers, DNA polymerase, and nucleic acid template. The PCR reaction comprises providing a set of polynucleotide primers wherein a first primer contains a sequence complementary to a region in one strand of the nucleic acid template sequence and primes the synthesis of a complementary DNA strand, and a second primer contains a sequence complementary to a region in a second strand of the target nucleic acid sequence and primes the synthesis of a complementary DNA strand, and amplifying the nucleic acid template sequence employing a nucleic acid polymerase as a template-dependent polymerizing agent under conditions which are permissive for PCR cycling steps of (i) annealing of primers required for amplification to a target nucleic acid sequence contained within the template sequence, (ii) extending the primers wherein the nucleic acid polymerase synthesizes a primer extension product. “A set of polynucleotide primers”, “a set of PCR primers” or “pair of primers” can comprise two, three, four or more primers.

Real time nucleic acid amplification and detection methods are efficient for sequence identification and quantification of a target since no pre-hybridization amplification is required. Amplification and hybridization are combined in a single step and can be performed in a fully automated, large-scale, closed-tube format.

Methods that use hybridization-triggered fluorescent probes for real time PCR are based either on a quench-release fluorescence of a probe digested by DNA Polymerase (e.g., methods using TaqMan(R), MGB-TaqMan(R)), or on a hybridization-triggered fluorescence of intact probes (e.g., molecular beacons, and linear probes). In general, the probes are designed to hybridize to an internal region of a PCR product during annealing stage (also referred to as amplicon). For those methods utilizing TaqMan(R) and MGB-TaqMan(R) the 5′-exonuclease activity of the approaching DNA Polymerase cleaves a probe between a fluorophore and a quencher, releasing fluorescence.

Thus, a “real time PCR” or “RT-PCT” assay provides dynamic fluorescence detection of amplified miR-146a, any of the miR-146a regulated genes or any control reference gene or miRNA produced in a PCR amplification reaction. During PCR, the amplified products created using suitable primers hybridize to probe nucleic acids (TaqMan(R) probe, for example), which may be labeled according to some embodiments with both a reporter dye and a quencher dye. When these two dyes are in close proximity, i.e. both are present in an intact probe oligonucleotide, the fluorescence of the reporter dye is suppressed. However, a polymerase, such as AmpliTaq Gold™, having 5′-3′ nuclease activity can be provided in the PCR reaction. This enzyme cleaves the fluorogenic probe if it is bound specifically to the target nucleic acid sequences between the priming sites. The reporter dye and quencher dye are separated upon cleavage, permitting fluorescent detection of the reporter dye. Upon excitation by a laser provided, e.g., by a sequencing apparatus, the fluorescent signal produced by the reporter dye is detected and/or quantified. The increase in fluorescence is a direct consequence of amplification of target nucleic acids during PCR. The method and hybridization assays using self-quenching fluorescence probes with and/or without internal controls for detection of nucleic acid application products are known in the art, for example, U.S. Pat. Nos. 6,258,569; 6,030,787; 5,952,202; 5,876,930; 5,866,336; 5,736,333; 5,723,591; 5,691,146; and 5,538,848.

More particularly, QRT-PCR or “qPCR” (Quantitative RT-PCR), which is quantitative in nature, can also be performed to provide a quantitative measure of gene expression levels. In QRT-PCR reverse transcription and PCR can be performed in two steps, or reverse transcription combined with PCR can be performed. One of these techniques, for which there are commercially available kits such as TaqMan(R) (Perkin Elmer, Foster City, Calif.), is performed with a transcript-specific antisense probe. This probe is specific for the PCR product (e.g. a nucleic acid fragment derived from a gene, or in this case, from a pre-miRNA) and is prepared with a quencher and fluorescent reporter probe attached to the 5′ end of the oligonucleotide. Different fluorescent markers are attached to different reporters, allowing for measurement of at least two products in one reaction.

When Taq DNA polymerase is activated, it cleaves off the fluorescent reporters of the probe bound to the template by virtue of its 5-to-3′ exonuclease activity. In the absence of the quenchers, the reporters now fluoresce. The color change in the reporters is proportional to the amount of each specific product and is measured by a fluorometer; therefore, the amount of each color is measured and the PCR product is quantified. The PCR reactions can be performed in any solid support, for example, slides, microplates, 96 well plates, 384 well plates and the like so that samples derived from many individuals are processed and measured simultaneously. The TaqMan(R) system has the additional advantage of not requiring gel electrophoresis and allows for quantification when used with a standard curve.

A second technique useful for detecting PCR products quantitatively without is to use an intercalating dye such as the commercially available QuantiTect SYBR Green PCR (Qiagen, Valencia Calif.). RT-PCR is performed using SYBR green as a fluorescent label which is incorporated into the PCR product during the PCR stage and produces fluorescence proportional to the amount of PCR product.

Both TaqMan(R) and QuantiTect SYBR systems can be used subsequent to reverse transcription of RNA. Reverse transcription can either be performed in the same reaction mixture as the PCR step (one-step protocol) or reverse transcription can be performed first prior to amplification utilizing PCR (two-step protocol).

Additionally, other known systems to quantitatively measure mRNA expression products include Molecular Beacons(R) which uses a probe having a fluorescent molecule and a quencher molecule, the probe capable of forming a hairpin structure such that when in the hairpin form, the fluorescence molecule is quenched, and when hybridized, the fluorescence increases giving a quantitative measurement of gene expression, or in this case, miRNA expression.

According to this embodiment, the detecting molecule may be in the form of probe corresponding and thereby hybridizing to any region or part of miR-146a, and at least one of miR-146a regulated genes or any control reference gene or miRNA. More particularly, it is important to choose regions which will permit hybridization to the target nucleic acids. Factors such as the Tm of the oligonucleotide, the percent GC content, the degree of secondary structure and the length of nucleic acid are important factors.

It should be further noted that a standard Northern blot assay can also be used to ascertain an RNA transcript size and the relative amounts of miR-146a and miR-146a regulated genes or any control gene product, in accordance with conventional Northern hybridization techniques known to those persons of ordinary skill in the art.

Particular embodiments of the method of the invention are based on detecting the expression values of miR-146a. According to this embodiment, the detecting nucleic acid molecules used by the method of the invention comprise isolated oligonucleotides that specifically hybridize to a nucleic acid sequence of miR-146a, and isolated oligonucleotides that specifically hybridize to a nucleic acid sequence of at least one of the control reference gene or miRNA.

Yet other embodiments of the method of the invention are based on detecting the expression values of miR-146a and at least one of miR-146a regulated genes. According to this embodiment, the detecting nucleic acid molecules used by the method of the invention comprise isolated oligonucleotides that specifically hybridize to a nucleic acid sequence of miR-146a, isolated oligonucleotides that specifically hybridize to a nucleic acid sequence of at least one of miR-146a regulated genes and isolated oligonucleotides that specifically hybridize to a nucleic acid sequence of at least one of the control reference gene or miRNA. It should be appreciated that all the detecting molecules used by any of the methods, as well as the compositions and kits of the invention described herein after, are isolated and/or purified molecules. As used herein, “isolated” or “purified” when used in reference to a nucleic acid means that a naturally occurring sequence has been removed from its normal cellular (e.g., chromosomal) environment or is synthesized in a non-natural environment (e.g., artificially synthesized). Thus, an “isolated” or “purified” sequence may be in a cell-free solution or placed in a different cellular environment. The term “purified” does not imply that the sequence is the only nucleotide present, but that it is essentially free (about 90-95 percent pure) of non-nucleotide material naturally associated with it, and thus is distinguished from isolated chromosomes.

As detailed above and as used herein the terms “miR-146a”, or any “control reference gene or miRNA” refer to the miRNA expressed by genes encoding miR-146a or any control reference gene or miRNA, and refers to the sequence of miR-146a or any control reference gene miRNA, including pri- and pre-miR-146a or any appropriate control reference gene or miRNA. It should be noted that the miRs sequences used by the present invention were obtained from miRBase. More specifically, the mature sequence: MIMAT0000449 of hsa-miR-146a comprises the nucleic acid sequence of: ugagaacuga auuccauggguu. In certain embodiments, said miR-146a is also denoted by SEQ ID NO. 1. It yet other embodiments, the pre-miRNA-146a sequence: MI0000477 comprises the nucleic acid sequence of ccgauguguauccucagcuu ugagaacuga auuccauggg uugugucagugucagaccucugaaauucaguucuucagcugggauaucucugucaucgu.

More specifically, said pre-miRNA-146a is also denoted by SEQ ID NO. 2.

The invention further contemplates the use of amino acid based molecules such as proteins or polypeptides as detecting molecules disclosed herein and would be known by a person skilled in the art to measure the protein products of the marker miR-146a regulated genes of the invention. Techniques known to persons skilled in the art (for example, techniques such as Western Blotting, Immunoprecipitation, ELISAs, protein microarray analysis, Flow cytometry and the like) can then be used to measure the level of protein products corresponding to the biomarker of the invention. As would be understood to a person skilled in the art, the measure of the level of expression of the protein products of the biomarker of the invention, specifically, miR-146a regulated genes, requires a protein, which specifically and/or selectively binds to the biomarker genes of the invention.

As indicated above, the detecting molecules of the invention may be amino acid based molecules that may be referred to as protein/s or polypeptide/s. As used herein, the terms “protein” and “polypeptide” are used interchangeably to refer to a chain of amino acids linked together by peptide bonds. In a specific embodiment, a protein is composed of less than 200, less than 175, less than 150, less than 125, less than 100, less than 50, less than 45, less than 40, less than 35, less than 30, less than 25, less than 20, less than 15, less than 10, or less than 5 amino acids linked together by peptide bonds. In another embodiment, a protein is composed of at least 200, at least 250, at least 300, at least 350, at least 400, at least 450, at least 500 or more amino acids linked together by peptide bonds. It should be noted that peptide bond as described herein is a covalent amid bond formed between two amino acid residues.

In specific embodiments, the detecting amino acid molecules are isolated antibodies, with specific binding selectively to the proteins encoded by miR-146a regulated genes as detailed above. Using these antibodies, the level of expression of proteins encoded by miR-146a regulated genes may be determined using an immunoassay which is selected from the group consisting of FACS, a Western blot, an ELISA, a RIA, a slot blot, a dot blot, immunohistochemical assay and a radio-imaging assay.

The term “antibody” as used in this invention includes whole antibody molecules as well as functional fragments thereof, such as Fab, F(ab′)2, and Fv that are capable of binding with antigenic portions of the target polypeptide, i.e. proteins encoded by miR-146a regulated genes. The antibody is preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition”, which as used herein refer to a preparation of antibodies or fragments thereof of single molecular composition.

It should be recognized that the antibody can be a human antibody, a chimeric antibody, a recombinant antibody, a humanized antibody, a monoclonal antibody, or a polyclonal antibody. The antibody can be an intact immuno globulin, e.g., an IgA, IgG, IgE, IgD, 1gM or subtypes thereof. The antibody can be conjugated to a functional moiety (e.g., a compound which has a biological or chemical function. The antibody used by the invention interacts with a polypeptide that is a product of any one of miR146a regulated genes, specifically, any one of IFI44L, MX2, RSAD2, IFIT5, IFITM1, IFITM3, IRF7, ISG15, IF127, TRAF6, IF144, IFIT3, OASL, TRIM22, IFIT1, IRAK1 and IRAK2, with high affinity and specificity.

As noted above, the term “antibody” also encompasses antigen-binding fragments of an antibody. The term “antigen-binding fragment” of an antibody (or simply “antibody portion,” or “fragment”), as used herein, may be defined as follows:
(1) Fab, the fragment which contains a monovalent antigen-binding fragment of an antibody molecule, can be produced by digestion of whole antibody with the enzyme papain to yield an intact light chain and a portion of one heavy chain;
(2) Fab′, the fragment of an antibody molecule that can be obtained by treating whole antibody with pepsin, followed by reduction, to yield an intact light chain and a portion of the heavy chain; two Fab′ fragments are obtained per antibody molecule;
(3) (Fab′)2, the fragment of the antibody that can be obtained by treating whole antibody with the enzyme pepsin without subsequent reduction; F(ab′)2 is a dimer of two Fab′ fragments held together by two disulfide bonds;
(4) Fv, defined as a genetically engineered fragment containing the variable region of the light chain and the variable region of the heavy chain expressed as two chains; and
(5) Single chain antibody (“SCA”, or ScFv), a genetically engineered molecule containing the variable region of the light chain and the variable region of the heavy chain, linked by a suitable polypeptide linker as a genetically fused single chain molecule.

Methods of generating such antibody fragments are well known in the art (See for example, Harlow and Lane, Antibodies: A Laboratory Manual, Cold Spring Harbor Laboratory, New York, 1988, incorporated herein by reference).

Purification of serum immunoglobulin antibodies (polyclonal antisera) or reactive portions thereof can be accomplished by a variety of methods known to those of skill in the art including, precipitation by ammonium sulfate or sodium sulfate followed by dialysis against saline, ion exchange chromatography, affinity or immuno-affinity chromatography as well as gel filtration, zone electrophoresis, etc.

Still further, for diagnostic and monitoring uses described herein after, the anti-proteins encoded by miR-146a regulated genes antibodies used by the present invention may optionally be covalently or non-covalently linked to a detectable label. The term “labeled” can refer to direct labeling of the antibody via, e.g., coupling (i.e., physically linking) a detectable substance to the antibody, and can also refer to indirect labeling of the antibody by reactivity with another reagent that is directly labeled. Examples of indirect labeling include detection of a primary antibody using a fluorescently labeled secondary antibody. More specifically, detectable labels suitable for such use include any composition detectable by spectroscopic, photochemical, biochemical, immunochemical, electrical, optical or chemical means. Useful labels in the present invention include magnetic beads (e.g. DYNABEADS), fluorescent dyes (e.g., fluorescein isothiocyanate, Texas red, rhodamine, green fluorescent protein, and the like), radiolabels (e.g., ³H, ¹²⁵I, ³⁵S, ¹⁴C, or ³²P), enzymes (e.g., horseradish peroxidase, alkaline phosphatase and others commonly used in an ELISA and competitive ELISA and other similar methods known in the art) and colorimetric labels such as colloidal gold or colored glass or plastic (e.g. polystyrene, polypropylene, latex, etc.) beads.

Means of detecting such labels are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

The antibody used as a detecting molecule according to the invention, specifically recognizes and binds proteins encoded by miR-146a regulated genes. It should be therefore noted that the term “binding specificity”, “specifically binds to an antigen”, “specifically immuno-reactive with”, “specifically directed against” or “specifically recognizes”, when referring to an epitope, specifically, a recognized epitope within the proteins encoded by miR-146a regulated genes, refers to a binding reaction which is determinative of the presence of the epitope in a heterogeneous population of proteins and other biologics. More particularly, “selectively bind” in the context of proteins encompassed by the invention refers to the specific interaction of a any two of a peptide, a protein, a polypeptide an antibody, wherein the interaction preferentially occurs as between any two of a peptide, protein, polypeptide and antibody preferentially as compared with any other peptide, protein, polypeptide and antibody.

Thus, under designated immunoassay conditions, the specified antibodies bind to a particular epitope at least two times the background and more typically more than 10 to 100 times background. More specifically, “Selective binding”, as the term is used herein, means that a molecule binds its specific binding partner with at least 2-fold greater affinity, and preferably at least 10-fold, 20-fold, 50-fold, 100-fold or higher affinity than it binds a non-specific molecule.

A variety of immunoassay formats may be used to select antibodies specifically immunoreactive with a particular protein or carbohydrate. For example, solid-phase ELISA immunoassays are routinely used to select antibodies specifically immunoreactive with a protein or carbohydrate. The term “epitope” is meant to refer to that portion of any molecule capable of being bound by an antibody which can also be recognized by that antibody. Epitopes or “antigenic determinants” usually consist of chemically active surface groupings of molecules such as amino acids or sugar side chains and have specific three dimensional structural characteristics as well as specific charge characteristics.

According to one embodiment, where amino acid-based detection molecules are used, the expression level of the proteins encoded by miR-146a regulated genes, in the tested sample can be determined using different methods known in the art, specifically method disclosed herein below as non-limiting examples.

Enzyme-Linked Immunosorbent Assay (ELISA) is used herein involves fixation of a sample containing a protein substrate (e.g., fixed cells or a proteinaceous solution) to a surface such as a well of a microtiter plate. A substrate-specific antibody coupled to an enzyme is applied and allowed to bind to the substrate. Presence of the antibody is then detected and quantitated by a colorimetric reaction employing the enzyme coupled to the antibody. Enzymes commonly employed in this method include horseradish peroxidase and alkaline phosphatase. If well calibrated and within the linear range of response, the amount of substrate present in the sample is proportional to the amount of color produced. A substrate standard is generally employed to improve quantitative accuracy.

Western Blot as used herein involves separation of a substrate from other protein by means of an acryl amide gel followed by transfer of the substrate to a membrane (e.g., nitrocellulose, nylon, or PVDF). Presence of the substrate is then detected by antibodies specific to the substrate, which are in turn detected by antibody-binding reagents. Antibody-binding reagents may be, for example, protein A or secondary antibodies. Antibody-binding reagents may be radio labeled or enzyme-linked, as described hereinafter. Detection may be by autoradiography, colorimetric reaction, or chemiluminescence. This method allows both quantization of an amount of substrate and determination of its identity by a relative position on the membrane indicative of the protein's migration distance in the acryl amide gel during electrophoresis, resulting from the size and other characteristics of the protein.

In one version, Radioimmunoassay (RIA) involves precipitation of the desired protein (i.e., the substrate) with a specific antibody and radio labeled antibody-binding protein (e.g., protein A labeled with I¹²⁵) immobilized on a perceptible carrier such as agars beads. The radio-signal detected in the precipitated pellet is proportional to the amount of substrate bound.

In an alternate version of RIA, a labeled substrate and an unlabelled antibody-binding protein are employed. A sample containing an unknown amount of substrate is added in varying amounts. The number of radio counts from the labeled substrate-bound precipitated pellet is proportional to the amount of substrate in the added sample.

Fluorescence-Activated Cell Sorting (FACS) involves detection of a substrate in situ in cells bound by substrate-specific, fluorescently labeled antibodies. The substrate-specific antibodies are linked to fluorophore. Detection is by means of a flow cytometry machine, which reads the wavelength of light emitted from each cell as it passes through a light beam. This method may employ two or more antibodies simultaneously, and is a reliable and reproducible procedure used by the present invention.

Immunohistochemical Analysis involves detection of a substrate in situ in fixed cells by substrate-specific antibodies. The substrate specific antibodies may be enzyme-linked or linked to fluorophore. Detection is by microscopy, and is either subjective or by automatic evaluation. With enzyme-linked antibodies, a calorimetric reaction may be required. It will be appreciated that immunohistochemistry is often followed by counterstaining of the cell nuclei, using, for example, Hematoxyline or Giemsa stain.

Still further, according to certain embodiments, the method of the invention uses any appropriate biological sample. The term “biological sample” in the present specification and claims is meant to include samples obtained from a mammal subject.

It should be recognized that in certain embodiments a biological sample may be for example, bone marrow, lymph fluid, blood cells, blood, serum, plasma, urine, sputum, saliva, faeces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents. More specific embodiments, the sample may be any one of peripheral blood mononuclear cells and biopsies of organs or tissues.

According to an embodiment of the invention, the sample is a cell sample. More specifically, the cell is a blood cell (e.g., white blood cells, macrophages, B- and T-lymphocytes, monocytes, neutrophiles, eosinophiles, and basophiles) which can be obtained using a syringe needle from a vein of the subject. It should be noted that the cell may be isolated from the subject (e.g., for in vitro detection) or may optionally comprise a cell that has not been physically removed from the subject (e.g., in vivo detection).

According to a specific embodiment, the sample used by the method of the invention is a sample of peripheral blood mononuclear cells (PBMCs).

The phrase, “peripheral blood mononuclear cells (PBMCs)” as used herein, refers to a mixture of monocytes and lymphocytes. Several methods for isolating white blood cells are known in the art. For example, PBMCs can be isolated from whole blood samples using density gradient centrifugation procedures. Typically, anticoagulated whole blood is layered over the separating medium. At the end of the centrifugation step, the following layers are visually observed from top to bottom: plasma/platelets, PBMCs, separating medium and erythrocytes/granulocytes. The PBMC layer is then removed and washed to remove contaminants (e.g., red blood cells) prior to determining the expression level of the polynucleotide(s) bio-markers of the invention.

In yet another embodiment, the sample may be a biopsy of human organs or tissue, specifically, liver biopsy.

According to some embodiments, the sample may be biopsies of organs or tissues. The biopsies may be obtained by a surgical operation from an organ or tissue of interest, for example liver biopsy, cerebrospinal fluid (CSF), brain biopsy, skin biopsy.

The term biopsy used herein refers to a medical test commonly performed by a surgeon or an interventional radiologist involving sampling of cells or tissues for examination. It is the medical removal of tissue from a living subject to determine the presence or extent of a disease. The tissue is generally examined under a microscope by a pathologist, and can also be analyzed chemically. When an entire lump or suspicious area is removed, the procedure is called an excisional biopsy. When only a sample of tissue is removed with preservation of the histological architecture of the tissue's cells, the procedure is called an incisional biopsy or core biopsy. When a sample of tissue or fluid is removed with a needle in such a way that cells are removed without preserving the histological architecture of the tissue cells, the procedure is called a needle aspiration biopsy.

According to some embodiments of the invention, the cell is a liver cell. It should be noted that liver cells (hepatic cell) can be obtained by a liver biopsy (e.g., using a surgical tool or a needle). It should be noted that certain embodiments of the invention contemplate the use of different biological samples.

The invention further encompasses the use of the miR-146a and at least one of miR-146a regulated genes of the invention as a biomarker for predicting, assessing and monitoring response to interferon treatment in subjects in need of interferon treatment. Such subject may be for example a subject suffering from an immune-related disorder.

It should be noted that an “Immune-related disorder” is a condition that is associated with the immune system of a subject, either through activation or inhibition of the immune system, or that can be treated, prevented or diagnosed by targeting a certain component of the immune response in a subject, such as the adaptive or innate immune response.

In specific embodiments, such immune-related disorder may be any one of an autoimmune disease, an infectious condition and a proliferative disorder.

A subset of immune-mediated diseases is known as autoimmune diseases. As used herein autoimmune diseases arise from an inappropriate immune response of the body against substances and tissues normally present in the body. In other words, the immune system mistakes some part of the body as a pathogen and attacks its own cells. This may be restricted to certain organs (e.g. in autoimmune thyroiditis) or involve a particular tissue in different places (e.g. Goodpasture's disease which may affect the basement membrane in both the lung and the kidney). Autoimmune disease are categorized by Witebsky's postulates (first formulated by Ernst Witebsky and colleagues in 1957) and include (i) direct evidence from transfer of pathogenic antibody or pathogenic T cells, (ii) indirect evidence based on reproduction of the autoimmune disease in experimental animals and (iii) circumstantial evidence from clinical clues. The treatment of autoimmune diseases is typically done by compounds that decrease the immune response.

Non-limiting examples for autoimmune disorders include Multiple Sclerosis (MS), inflammatory arthritis. rheumatoid arthritis (RA), Eaton-Lambert syndrome, Goodpasture's syndrome, Greave's disease, Guillain-Barr syndrome, autoimmune hemolytic anemia (AIHA), hepatitis, insulin-dependent diabetes mellitus (IDDM) and NIDDM, systemic lupus erythematosus (SLE), myasthenia gravis, plexus disorders e.g. acute brachial neuritis, polyglandular deficiency syndrome, primary biliary cirrhosis, rheumatoid arthritis, scleroderma, thrombocytopenia, thyroiditis e.g. Hashimoto's disease, Sjogren's syndrome, allergic purpura, psoriasis, mixed connective tissue disease, polymyositis, dermatomyositis, vasculitis, polyarteritis nodosa, arthritis, alopecia areata, polymyalgia rheumatica, Wegener's granulomatosis, Reiter's syndrome, Behget's syndrome, ankylosing spondylitis, pemphigus, bullous pemphigoid, dermatitis herpetiformis, inflammatory bowel disease, ulcerative colitis and Crohn's disease and fatty liver disease.

As shown in Examples 1 and 3, the levels of miR-146a regulated genes are differently expressed in different stages of MS. Thus, in more specific embodiment, the method of the invention may be particularly useful for predicting responsiveness to interferon treatment in a subject suffering from an autoimmune disorder, specifically, Multiple sclerosis (MS).

As used herein the phrase “multiple sclerosis” (abbreviated MS, formerly known as disseminated sclerosis or encephalomyelitis disseminata) is a chronic, inflammatory, demyelinating disease that affects the central nervous system (CNS). Disease onset usually occurs in young adults, is more common in women, and has a prevalence that ranges between 2 and 150 per 100,000 depending on the country or specific population. MS is characterized by presence of at least two neurological attacks affecting the central nervous system (CNS) and accompanied by demyelinating lesions on brain magnetic resonance imaging (MRI). MS takes several forms, with new symptoms occurring either in discrete episodes (relapsing forms) or slowly accumulating over time (progressive forms). Most people are first diagnosed with relapsing-remitting MS (RRMS) but develop secondary-progressive MS (SPMS) after a number of years. Between episodes or attacks, symptoms may go away completely, but permanent neurological problems often persist, especially as the disease advances.

Relapsing-remitting multiple sclerosis (RRMS) occurring in 85 percent of the patients and a progressive multiple sclerosis occurring in 15 percent of the patients.

According to some embodiments of the invention, the method of the invention may be particularly applicable for subjects diagnosed with RRMS, where early diagnosis of relapse may improve the treatment.

In yet another embodiment, the method of the invention may be suitable for predicting responsiveness to interferon treatment in a subject suffering from an inflammatory disorder, specifically, an infectious condition caused by a pathogenic agent. More specifically, such infectious conditions may be any one of viral diseases, protozoan diseases, bacterial diseases, parasitic diseases, fungal diseases and mycoplasma diseases.

It should be appreciated that an infectious disease as used herein also encompasses any infectious disease caused by a pathogenic agent. Pathogenic agents include prokaryotic microorganisms, lower eukaryotic microorganisms, complex eukaryotic organisms, viruses, fungi, prions, parasites and yeasts.

A prokaryotic microorganism includes bacteria such as Gram positive, Gram negative and Gram variable bacteria and intracellular bacteria. Examples of bacteria contemplated herein include the species of the genera Treponema sp., Borrelia sp., Neisseria sp., Legionella sp., Bordetella sp., Escherichia sp., Salmonella sp., Shigella sp., Klebsiella sp., Yersinia sp., Vibrio sp., Hemophilus sp., Rickettsia sp., Chlamydia sp., Mycoplasma sp., Staphylococcus sp., Streptococcus sp., Bacillus sp., Clostridium sp., Corynebacterium sp., Proprionibacterium sp., Mycobacterium sp., Ureaplasma sp. and Listeria sp.

Particular species include Treponema pallidum, Borrelia burgdorferi, Neisseria gonorrhea, Neisseria meningitidis, Legionella pneumophila, Bordetella pertussis, Escherichia coli, Salmonella typhi, Salmonella typhimurium, Shigella dysenteriae, Klebsiella pneumoniae, Yersinia pestis, Vibrio cholerae, Hemophilus influenzae, Rickettsia rickettsii, Chlamydia trachomatis, Mycoplasma pneumoniae, Staphylococcus aureus, Streptococcus pneumoniae, Streptococcus pyogenes, Bacillus anthracis, Clostridium botulinum, Clostridium tetani, Clostridium perfringens, Corynebacterium diphtheriae, Proprionibacterium acnes, Mycobacterium tuberculosis, Mycobacterium leprae and Listeria monocytogenes.

A lower eukaryotic organism includes a yeast or fungus such as but not limited to Pneumocystis carinii, Candida albicans, Aspergillus, Histoplasma capsulatum, Blastomyces dermatitidis, Cryptococcus neoformans, Trichophyton and Microsporum.

A complex eukaryotic organism includes worms, insects, arachnids, nematodes, aemobe, Entamoeba histolytica, Giardia lamblia, Trichomonas vaginalis, Trypanosoma brucei gambiense, Trypanosoma cruzi, Balantidium coli, Toxoplasma gondii, Cryptosporidium or Leishmania.

The term “fungi” includes for example, fungi that cause diseases such as ringworm, histoplasmosis, blastomycosis, aspergillosis, cryptococcosis, sporotrichosis, coccidioidomycosis, paracoccidio-idoinycosis, and candidiasis.

The term parasite includes, but not limited to, infections caused by somatic tapeworms, blood flukes, tissue roundworms, ameba, and Plasmodium, Trypanosoma, Leishmania, and Toxoplasma species.

The term “viruses” is used in its broadest sense to include viruses of the families adenoviruses, papovaviruses, herpesviruses: simplex, varicella-zoster, Epstein-Barr, CMV, pox viruses: smallpox, vaccinia, hepatitis B, rhinoviruses, hepatitis A, poliovirus, rubella virus, hepatitis C, arboviruses, rabies virus, influenza viruses A and B, measles virus, mumps virus, HIV, HTLV I and II.

As shown by Examples 5 and 6, the biomarkers used by method of the invention distinguish between interferon responders and non-responders HCV infected subjects. Therefore, the method of the invention may be used for predicting interferon responsiveness in subjects suffering from viral infections, for example, Hepatitis C virus infection (type 1, 2, 3 or 4), or HCV or influenza infections.

In specific embodiments, the infectious condition may be hepatitis C virus (HCV) infection.

As used herein the term “HCV” refers to hepatitis C virus having genotype 1 (also known as HCV Type 1), genotype 2 (also known as HCV Type 2), genotype 3 (also known as HCV Type 3), genotype 4 (also known as HCV Type 4), genotype 5 (also known as HCV Type 5) or genotype 6 (also known as HCV Type 6).

The phrase “HCV infection” encompasses acute (refers to the first 6 months after infection) and chronic (refers to infection with hepatitis C virus which persists more than 6 month) infection with the hepatitis C virus. Thus, according to some embodiments of the invention, the subject is diagnosed with chronic HCV infection. According to some embodiments of the invention, the subject is infected with HCV type 1. According to some embodiments of the invention, the subject is infected with HCV type 2, 3 or 4.

As shown by Example 6, the method of the invention may be applicable for predicting responsiveness for interferon treatment in subjects suffering from influenza infections. Thus, in specific embodiments, the infectious condition is a virus of the Orthomyxoviridae, family, such as, but not limited to, Influenza virus A, Influenza virus B, Influenza virus C or any subtype and reassortants thereof.

As used herein the term Influenza viruses refers to orthomyxoviruses, and fall into three types; A, B and C. Influenza A and B virus particles contain a genome of negative sense, single-strand RNA divided into 8 linear segments. Co-infection of a single host with two different influenza viruses may result in the generation of reassortant progeny viruses having a new combination of genome segments, derived from each of the parental viruses Influenza A viruses have been responsible for four recent pandemics of severe human respiratory illness.

Type A influenza viruses are divided into subtypes based on two proteins on the surface of the virus, hemagglutinin (HA) and neuraminidase (NA). There are 15 different HA subtypes and 9 different NA subtypes. Subtypes of influenza A virus are named according to their HA and NA surface proteins. For example, an “H7N2 virus” designates influenza A subtype that has an HA 7 protein and an NA 2 protein. Similarly an “H5N1” virus has an HA 5 protein and an NA 1 protein. “Human flu viruses” are those subtypes that occur widely in humans. There are only three known A subtypes of human flu viruses (H1N1, H2N2, and H3N2). All known subtypes of A viruses can be found in birds. Symptoms of human infection with avian viruses have ranged from typical flu-like symptoms (fever, cough, sore throat and muscle aches) to eye infections, pneumonia, severe respiratory diseases (such as acute respiratory distress), and other severe and life-threatening complications.

As shown by Example 4, the levels of miR146a are elevated in subjects suffering from multiple melanoma. Thus, according to specific embodiments, the method of the invention may be suitable for subjects suffering from a proliferative disorder, specifically, any one of melanoma, carcinoma sarcoma, glioma, leukemia and lymphoma.

It should be noted that a proliferative disorder as used herein, encompasses malignant and non-malignant proliferative disorders.

As used herein to describe the present invention, “cancer”, “tumor” and “malignancy” all relate equivalently to a hyperplasia of a tissue or organ. If the tissue is a part of the lymphatic or immune systems, malignant cells may include non-solid tumors of circulating cells. Malignancies of other tissues or organs may produce solid tumors. In general, the methods of the present invention may be applicable for predicting, assessing and monitoring the response of patients suffering of non-solid and solid tumors to interferon treatment.

Malignancy, as contemplated in the present invention may be any one of melanomas, carcinomas, lymphomas, leukemias, myeloma and sarcomas.

Melanoma as used herein and will be described in more detail hereinafter, is a malignant tumor of melanocytes. Melanocytes are cells that produce the dark pigment, melanin, which is responsible for the color of skin. They predominantly occur in skin, but are also found in other parts of the body, including the bowel and the eye. Melanoma can occur in any part of the body that contains melanocytes.

Carcinoma as used herein, refers to an invasive malignant tumor consisting of transformed epithelial cells. Alternatively, it refers to a malignant tumor composed of transformed cells of unknown histogenesis, but which possess specific molecular or histological characteristics that are associated with epithelial cells, such as the production of cytokeratins or intercellular bridges.

Leukemia refers to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic).

Sarcoma is a cancer that arises from transformed connective tissue cells. These cells originate from embryonic mesoderm, or middle layer, which forms the bone, cartilage, and fat tissues. This is in contrast to carcinomas, which originate in the epithelium. The epithelium lines the surface of structures throughout the body, and is the origin of cancers in the breast, colon, and pancreas.

Myeloma as mentioned herein, is a cancer of plasma cells, a type of white blood cell normally responsible for the production of antibodies. Collections of abnormal cells accumulate in bones, where they cause bone lesions, and in the bone marrow where they interfere with the production of normal blood cells. Most cases of myeloma also feature the production of a paraprotein, an abnormal antibody that can cause kidney problems and interferes with the production of normal antibodies leading to immunodeficiency. Hypercalcemia (high calcium levels) is often encountered.

Lymphoma is a cancer in the lymphatic cells of the immune system. Typically, lymphomas present as a solid tumor of lymphoid cells. These malignant cells often originate in lymph nodes, presenting as an enlargement of the node (a tumor). It can also affect other organs in which case it is referred to as extranodal lymphoma.

Further malignancies that may find utility in the present invention can comprise but are not limited to hematological malignancies (including lymphoma, leukemia and myeloproliferative disorders), hypoplastic and aplastic anemia (both virally induced and idiopathic), myelodysplastic syndromes, all types of paraneoplastic syndromes (both immune mediated and idiopathic) and solid tumors (including GI tract, colon, lung, liver, breast, prostate, pancreas and Kaposi's sarcoma). More particularly, the malignant disorder may be lymphoma. Non-limiting examples of cancers treatable according to the invention include hematopoietic malignancies such as all types of lymphomas, leukemia, e.g. acute lymphocytic leukemia (ALL), acute myelogenous leukemia (AML), chronic lymphocytic leukemia (CLL), chronic myelogenous leukemia (CML), myelodysplastic syndrome (MDS), mast cell leukemia, hairy cell leukemia, Hodgkin's disease, non-Hodgkin's lymphomas, Burkitt's lymphoma and multiple myeloma, as well as for the treatment or inhibition of solid tumors such as tumors in lip and oral cavity, pharynx, larynx, paranasal sinuses, major salivary glands, thyroid gland, esophagus, stomach, small intestine, colon, colorectum, anal canal, liver, gallbladder, extraliepatic bile ducts, ampulla of vater, exocrine pancreas, lung, pleural mesothelioma, bone, soft tissue sarcoma, carcinoma and malignant melanoma of the skin, breast, vulva, vagina, cervix uteri, corpus uteri, ovary, fallopian tube, gestational trophoblastic tumors, penis, prostate, testis, kidney, renal pelvis, ureter, urinary bladder, urethra, carcinoma of the eyelid, carcinoma of the conjunctiva, malignant melanoma of the conjunctiva, malignant melanoma of the uvea, retinoblastoma, carcinoma of the lacrimal gland, sarcoma of the orbit, brain, spinal cord, vascular system, hemangiosarcoma and Kaposi's sarcoma.

As noted above, Example 4 demonstrates the feasibility of using miR-146a as a biomarker for melanoma patients. Thus, in one specific embodiment, the prognostic method of the invention may be used for predicting, assessing and monitoring the response of patient suffering from melanoma to interferon treatment. The term melanoma includes, but is not limited to, melanoma, metastatic melanoma, melanoma derived from either melanocytes or melanocyte-related nevus cells, melanocarcinoma, melanoepithelioma, melanosarcoma, melanoma in situ, superficial spreading melanoma, nodular melanoma, lentigo maligna melanoma, acral lentiginoous melanoma, invasive melanoma or familial atypical mole and melanoma (FAM-M) syndrome. Such melanomas may be caused by chromosomal abnormalities, degenerative growth and developmental disorders, mitogenic agents, ultraviolet radiation (UV), viral infections, inappropriate tissue gene expression, alterations in gene expression, or carcinogenic agents. The aforementioned melanomas can be treated by the method and the composition described in the present invention.

The invention further encompasses the use of the miR-146a and at least one of miR-146a regulated genes of the invention as a biomarker for predicting, assessing and monitoring the response to interferon treatment in subjects suffering from any condition related to the conditions described above. It is understood that the interchangeably used terms “linked”, “associated” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition” and the like, as they relate to a subject's health, are used interchangeably and have meanings ascribed to each and all of such terms.

In yet other alternative embodiments, determining the level of expression of miR-146a may further comprise detecting the presence of a single-nucleotide polymorphism (SNP) in at least one of immature or mature miR-146a.

A single-nucleotide polymorphism (SNP) as used herein encompasses a variation in the DNA sequence occurring when a single nucleotide—A, T, C or G—in the genome (or other shared sequence) differs between members of a biological species or paired chromosomes in an individual. For example, two sequenced DNA fragments from different individuals, AAGCCTA to AAGCTTA, contain a difference in a single nucleotide. In this case we say that there are two alleles: C and T.

For example in miR-146a, it has been previously found (Jazdzewski et al. (2008)) that the rarer C allele of a common G/C SNP (rs2910164) within the pre-miR-146a sequence reduced the amount of pre- and mature miR146A 1.9- and 1.8-fold, respectively, compared with the G allele. The SNP was reported to be located on the passenger strand of pre-miR146A, at position+60 relative to the first nucleotide, and the C allele is predicted to cause mispairing within the hairpin.

EMSA experiments showed that the C allele interfered with binding of HeLa cell nuclear proteins to pre-miR146a, and it also caused inefficient inhibition of the miR146a target genes TRAF6 and IRAK1, as well as of PTC1 (CCDC6; 601985), in reporter gene assays. Jazdzewski et al. (2008) genotyped 608 patients with papillary thyroid carcinoma (PTC; 188550) and 901 controls and found that GC heterozygosity was associated with increased risk of acquiring PTC, whereas both homozygous states were protective. They concluded that the G/C SNP alters pre-miR146a processing and contributes to predisposition to PTC by altering expression of miR146a target genes in the Toll-like receptor and cytokine signaling pathway.

A second aspect of the invention relates to a prognostic composition comprising:

(a) detecting molecules specific for determining the level of expression of miR-146a (denoted by SEQ ID NO:1) in a biological sample; and
(b) detecting molecules specific for determining the level of expression of at least one of miR-146a regulated genes (as provided in Table 1 in the Examples) in a biological sample. In an optional embodiment, the detecting molecules of (a) and (b) may be attached to a solid support.

According to one embodiment, the prognostic composition of the invention is particularly useful for predicting, assessing and monitoring responsiveness of a mammalian subject to interferon treatment.

In certain embodiments, the prognostic composition of the invention comprises detecting molecules that are selected from isolated detecting nucleic acid molecules and isolated detecting amino acid molecules.

In other embodiments the detecting molecules comprise isolated oligonucleotides, each oligonucleotide specifically hybridizes to a nucleic acid sequence of miR-146a or of at least one of miR-146a regulated genes and optionally, to a control miRNA or control reference gene.

More specifically, the detecting molecules may be at least one of a pair of primers or nucleotide probes. It should be appreciated that the different combinations of the detecting molecules used by the prognostic methods of the invention, are also applicable for any aspect disclosed by the invention, including the compositions and kits described herein after.

In certain embodiments, the compositions of the invention may further comprise detecting molecules specific for control reference gene or miRNA. Such miRNAs may be used for normalizing the detected expression levels for miR-146a and at least one of miR-146a regulated genes.

In one embodiment, the polynucleotide-based detection molecules of the invention may be in the form of nucleic acid probes which can be spotted onto an array to measure RNA from the sample of a subject to be diagnosed.

As defined herein, a “nucleic acid array” refers to a plurality of nucleic acids (or “nucleic acid members”), optionally attached to a support where each of the nucleic acid members is attached to a support in a unique pre-selected and defined region. These nucleic acid sequences are used herein as detecting nucleic acid molecules. In one embodiment, the nucleic acid member attached to the surface of the support is DNA. In a preferred embodiment, the nucleic acid member attached to the surface of the support is either cDNA or oligonucleotides. In another embodiment, the nucleic acid member attached to the surface of the support is cDNA synthesized by polymerase chain reaction (PCR). In another embodiment, a “nucleic acid array” refers to a plurality of unique nucleic acid detecting molecules attached to nitrocellulose or other membranes used in Southern and/or Northern blotting techniques. For oligonucleotide-based arrays, the selection of oligonucleotides corresponding to the gene of interest which are useful as probes is well understood in the art.

As indicated above, assay based on micro array or RT-PCR may involve attaching or spotting of the probes in a solid support. As used herein, the terms “attaching” and “spotting” refer to a process of depositing a nucleic acid onto a substrate to form a nucleic acid array such that the nucleic acid is stably bound to the substrate via covalent bonds, hydrogen bonds or ionic interactions.

As used herein, “stably associated” or “stably bound” refers to a nucleic acid that is stably bound to a solid substrate to form an array via covalent bonds, hydrogen bonds or ionic interactions such that the nucleic acid retains its unique pre-selected position relative to all other nucleic acids that are stably associated with an array, or to all other pre-selected regions on the solid substrate under conditions in which an array is typically analyzed (i.e., during one or more steps of hybridization, washes, and/or scanning, etc.).

As used herein, “substrate” or “support” or “solid support”, when referring to an array, refers to a material having a rigid or semi-rigid surface. The support may be biological, non-biological, organic, inorganic, or a combination of any of these, existing as particles, strands, precipitates, gels, sheets, tubing, spheres, beads, containers, capillaries, pads, slices, films, plates, slides, chips, etc. Often, the substrate is a silicon or glass surface, (poly)tetrafluoroethylene, (poly) vinylidendifmoride, polystyrene, polycarbonate, a charged membrane, such as nylon or nitrocellulose, or combinations thereof. Preferably, at least one surface of the substrate will be substantially flat. The support may optionally contain reactive groups, including, but not limited to, carboxyl, amino, hydroxyl, thiol, and the like. In one embodiment, the support may be optically transparent. As noted above, the solid support may include polymers, such as polystyrene, agarose, sepharose, cellulose, glass, glass beads and magnetizable particles of cellulose or other polymers. The solid-support can be in the form of large or small beads, chips or particles, tubes, plates, or other forms.

According to certain embodiments, the level of expression of at least one of said miR-146a or of at least one of miR-146a regulated genes may be determined using a nucleic acid amplification assay selected from the group consisting of: a Real-Time PCR, micro arrays, PCR, in situ Hybridization and Comparative Genomic Hybridization. It should be noted that the nucleic acid based procedures described herein for the prognostic methods of the invention may be applicable also for any of the aspects of the invention.

In yet other alternative embodiments, the composition of the invention may comprise detecting amino acid molecules such as isolated antibodies, each antibody binds selectively to a protein product of at least one of said at least one of miR-146a regulated genes. In such embodiments, the level of expression of the at least one miR-146a regulated genes may be determined using an immunoassay selected from the group consisting of an ELISA, a RIA, a slot blot, a dot blot, immunohistochemical assay, FACS, a radio-imaging assay and a Western blot.

As explained earlier, the inventors have analyzed the expression values of miR-146a and miR-146a regaled genes and found that changes in the expression level of the above are indicative of an increased likelihood for respond to interferon treatment and to be in a relapse stage.

As indicated herein before, the compositions and methods of the invention are particularly intended for predicting assessing and monitoring response to interferon treatment in a subject suffering from a disease treated with interferon.

In certain embodiments, the prognostic compositions of the invention are particularly suitable for use according to the prognostic method of the invention.

Thus, the invention further provides compositions for use in the prognosis of disease treated with interferon as well as monitoring and predicting responsiveness to interferon treatment and early diagnosis of relapse.

It should be appreciated that the composition of the invention may be used for predicating response of a mammalian subject to interferon treatment. According to one embodiment of the composition of the invention, the composition may be used to perform the prognostic method of the invention using a test sample of the subject obtained during diagnosis of a disease. The expression value of miR-146a and optionally of at least one of miR-146a regulated genes obtained from the examined sample is compared to a predetermined standard expression value or cutoff value. A positive expression value, or in other words, a higher expression value of the biomarker of the invention miR146a and optionally of at least one of miR-146a regulated genes, as compared to the predetermined standard expression value (cutoff value), indicates that said subject belongs to a pre-established population associated with lack of responsiveness to interferon treatment and therefore, the subject may be considered as a non-responsive subject.

It should be appreciated that the composition of the invention may be used for assessing responsiveness of a mammalian subject to interferon treatment or evaluating the efficacy of interferon treatment on a subject and for diagnosis of relapse.

Furthermore, in another embodiment of the composition of the invention, the composition may be used according to the prognostic method of the invention using at least two test samples of the subject, preferably three or more samples, wherein the samples are collected at different times from the subject.

The at least two time points are adjusted such that the required information is obtained. For example, in order to asses responsiveness to treatment, the first time point is before initiation of treatment and the second time point is at any time after initiation of treatment.

For example, in order to determine relapse, the at least two time points are obtained after initiation of treatment, preferably one of the time points is at remission.

The rate of change of the normalized expression values of miR-146a and at least one of miR-146a regulated genes between said temporally-separated test samples is being calculated.

The composition of the invention may therefore facilitate the prediction of probability of a patient to respond to interferon treatment, the monitoring and early sub-symptomatic diagnosis or prediction of a relapse in a subject when used according to the method of the invention for analysis of more than a single sample along the time-course of diagnosis, treatment and follow-up.

In yet another aspect, the invention provides a kit comprising: (a) detecting molecules specific for determining the level of expression of miR-146a in a biological sample; and (b) detecting molecules specific for determining the level of expression of at least one of miR-146a regulated genes in a biological sample. In certain embodiments, the kit of the invention may optionally further comprises at least one of:

(c) pre-determined calibration curve providing standard expression values of at least one of miR-146a and of at least one of miR-146a regulated genes; and (d) at least one control sample.

It should be noted that in certain embodiments, the control sample may be either a “negative” or a “positive” control. A “negative” or “positive” control is dependent upon the use of the kit.

According to another embodiment, the kit of the invention may be a prognostic kit for predicting, assessing and monitoring responsiveness of a mammalian subject to interferon treatment.

According to another embodiment, the kit of the invention may further comprise instructions for use. In more specific embodiments, such instructions comprises may include at least one of: (a) instructions for carrying out the detection and quantification of expression of said at least one of miR-146a or said at least one miR-146a regulated gene and optionally, of the control reference miRNA or a control reference gene; and (b) instructions for comparing the expression values of at least one of said miR-146a and at least one of miR-146a regulated genes with a corresponding predetermined standard expression value.

In yet other specific embodiments the kit of the invention may comprise detecting molecules specific for miR-146a regulated genes. In more specific embodiments, such miR-146a regulated genes may be selected from a group consisting of IFI44L, MX2, RSAD2, IFIT5, IFITM1, IFITM3, IRF7, ISG15, IF127, TRAF6, IF144, IFIT3, OASL, TRIM22, IFIT1, IRAK1 and IRAK2.

According to another embodiment the detecting molecules comprised in the kit of the invention may be isolated detecting nucleic acid molecules, isolated detecting amino acid molecules or any combinations thereof.

In more specific embodiments, the kit of the invention may comprise nucleic acid based detecting molecules, specifically, isolated oligonucleotides, each oligonucleotide specifically hybridize to a nucleic acid sequence of miR-146a or of at least one of miR-146a regulated genes. In an optional embodiment, the kit of the invention may further comprise nucleic acid based detecting molecules specific for a control miRNA or control reference gene. Such control gene or miRs may be used for normalizing the expression value measured in a specific test sample.

In yet other specific embodiments, the detecting molecules comprised in the kit of the invention may be at least one of a pair of primers or nucleotide probes.

In optional embodiments, the kit of the invention may further comprise at least one reagent for conducting a nucleic acid amplification based assay selected from the group consisting of a Real-Time PCR, micro arrays, PCR, in situ Hybridization and Comparative Genomic Hybridization.

According to certain embodiments, the kit of the invention is particularly suitable for predicting, assessing and monitoring response to interferon treatment in a subject diagnosed with a disease. According to specific embodiments, the disease to be treated may be any one of an autoimmune disease, a proliferative disorder and an infectious disease.

According to certain embodiments, the autoimmune disease may be multiple sclerosis.

According to another embodiment, the kit of the invention may be applicable in cases that the tested subject is suffering from a proliferative disorder, for example, any one of melanoma, carcinoma sarcoma, glioma, leukemia and lymphoma. More specific embodiments relate to melanoma.

Still further, in certain embodiments, the infectious disease is any one of protozoan diseases, viral diseases, bacterial diseases, parasitic diseases, fungal diseases and mycoplasma diseases. In a specific embodiment, the infectious disease is viral disease infection. In more specific embodiments, the viral infection is hepatitis C or influenza.

It should be appreciated that the kit of the invention is suitable for determining the expression level of miR-146a and miR-146a regulated genes in a biological sample. In some embodiments the biological sample may be any one of a blood cells, blood, bone marrow, lymph fluid, serum, plasma, urine, sputum, saliva, faeces, semen, spinal fluid or CSF, the external secretions of the skin, respiratory, intestinal, and genitourinary tracts, tears, milk, any human organ or tissue, any sample obtained by lavage, optionally of the breast ducal system, plural effusion, sample of in vitro or ex vivo cell culture and cell culture constituents.

According to specific embodiments, the biological sample may be a blood sample. Specifically, the biological sample is a sample of peripheral blood mononuclear cells (PBMCs). The kit of the invention may therefore optionally comprise suitable mans for obtaining said sample. More specifically, for using the kit of the invention, one must first obtain samples from the tested subjects. To do so, means for obtaining such samples may be required. Such means for obtaining a sample from the mammalian subject can be by any means for obtaining a sample from the subject known in the art. Examples for obtaining e.g. blood or bone marrow samples are known in the art and could be any kind of finger or skin prick or lancet based device, which basically pierces the skin and results in a drop of blood being released from the skin. In addition, aspirating or biopsy needles may be also used for obtaining spleen lymph nodes tissue samples. Samples may of course be taken from any other living tissue, or body secretions comprising viable cells, such as biopsies, saliva or even urine.

It should be appreciated that the kit of the invention may be applicable for assessing and monitoring responsiveness of a subject suffering from a condition to a treatment with interferon. In such case, the kit may further comprise as a further element (g), instructions for calculating the rate of change of the expression values (preferably, normalized values) of said miR-146a and miR-146a regulated genes between said temporally-separated test samples. It should be noted that a positive rate of change of said expression values in a sample obtained after initiation of said treatment as compared to the miR-146a and miR-146a regulated genes expression value in a sample obtained prior to initiation of said treatment, is indicative of the responsiveness of said subject to said treatment.

The inventors consider the kit of the invention in compartmental form. It should be therefore noted that the detecting molecules used for detecting the expression levels of miR-146a and miR-146a regulated genes may be provided in a kit attached to an array. As defined herein, a “detecting molecule array” refers to a plurality of detection molecules that may be nucleic acids based or protein based detecting molecules (specifically, probes, primers and antibodies), optionally attached to a support where each of the detecting molecules is attached to a support in a unique pre-selected and defined region.

For example, an array may contain different detecting molecules, such as specific antibodies or primers. As indicated herein before, in case a combined detection of miR-146a and miR-146a regulated genes expression level, the different detecting molecules for each target may be spatially arranged in a predetermined and separated location in an array. For example, an array may be a plurality of vessels (test tubes), plates, micro-wells in a micro-plate, each containing different detecting molecules, specifically, probes, primers and antibodies, against polypeptides encoded by the miR-146a regulated genes. An array may also be any solid support holding in distinct regions (dots, lines, columns) different and known, predetermined detecting molecules.

As used herein, “solid support” is defined as any surface to which molecules may be attached through either covalent or non-covalent bonds. Thus, useful solid supports include solid and semi-solid matrixes, such as aero gels and hydro gels, resins, beads, biochips (including thin film coated biochips), micro fluidic chip, a silicon chip, multi-well plates (also referred to as microtiter plates or microplates), membranes, filters, conducting and no conducting metals, glass (including microscope slides) and magnetic supports. More specific examples of useful solid supports include silica gels, polymeric membranes, particles, derivative plastic films, glass beads, cotton, plastic beads, alumina gels, polysaccharides such as Sepharose, nylon, latex bead, magnetic bead, paramagnetic bead, super paramagnetic bead, starch and the like. This also includes, but is not limited to, microsphere particles such as Lumavidin™ Or LS-beads, magnetic beads, charged paper, Langmuir-Blodgett films, functionalized glass, germanium, silicon, PTFE, polystyrene, gallium arsenide, gold, and silver. Any other material known in the art that is capable of having functional groups such as amino, carboxyl, thiol or hydroxyl incorporated on its surface, is also contemplated. This includes surfaces with any topology, including, but not limited to, spherical surfaces and grooved surfaces.

It should be further appreciated that any of the reagents, substances or ingredients included in any of the methods and kits of the invention may be provided as reagents embedded, linked, connected, attached, placed or fused to any of the solid support materials described above.

According to another aspect, the invention provides a method for treating, preventing, ameliorating or delaying the onset of an immune-related disorder in a subject. More specifically, the method of the invention may comprise the step of: (a) predicting, assessing and monitoring responsiveness of the tested subject to interferon treatment according to the method of the invention; and (b) selecting an interferon treatment regimen based on said responsiveness thereby treating said subject.

In still a further aspect, the invention provides a method for treating, preventing, ameliorating or delaying the onset of an immune-related disorder in a subject treated with interferon by modulating of the expression of miR-146a, the method comprising the step of administering to said subject a therapeutically effective amount of any one of: (a) antisense specific for miR-146a; (b) siRNA specific for miR-146a; and (c) miR-146a oligonucleotide or any composition comprising the same. In case that down-regulation of miR-146a regulated genes is desired, up-regulation of miR-146a expression may be achieved by administering miR-146a oligonucleotide or any composition comprising the same.

Optionally the method of treatment provided by the invention may include up-regulating the expression of at least one of miR-146a regulated genes.

According to specific embodiments, modulation of miR-146a expression may lead to any one of increasing or decreasing the expression of miR-146a.

The terms “decrease”, “inhibition”, “moderation” or “attenuation” as referred to herein, relate to the retardation, restraining or reduction of miR-146a and at least one of miR-146a regulated genes expression or levels by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

The terms “increase”, “elevation”, “enhancement” or “elevation” as referred to herein, relate to the enhancement and increase of miR-146a and at least one of miR-146a regulated genes expression or levels by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

According to specific embodiments, modulation of miR-146a regulated genes expression may lead either to an increase or decrease in the expression or the intracellular, extracellular or serum levels of polypeptide coded by miR-146a regulated genes or any one of increasing or decreasing the expression of miR-146a regulated genes.

According to one specific embodiment, where an increase in the expression of miR-146a is desired, the compound used by the method of the invention increases miR-146a expression.

According to one specific embodiment, where an increase in the expression or the intracellular, extracellular or serum levels of polypeptide encoded by miR-146a regulated genes is desired, the compound used by the method of the invention increases miR-146a regulated genes expression.

Alternatively, according to another specific embodiment, where a decrease in the expression of miR-146a is desired, the compound used by the method of the invention may decrease miR-146a expression. Similarly, according to another specific embodiment, where a decrease in the expression or the intracellular, extracellular or serum levels of polypeptide encoded by miR-146a regulated genes is desired, the compound used by the method of the invention may reduce miR-146a regulated genes expression.

The method of the invention involves administration of therapeutically effective amount of any one of: (a) antisense specific for miR-146a; (b) siRNA specific for miR-146a; that reduce miR146a levels or alternatively, (c) miR-146a oligonucleotide that modulates, specifically increase its expression and levels. The term “effective amount” as used herein is that determined by such considerations as are known to the man of skill in the art. The amount must be sufficient to prevent or ameliorate immune-related disorders, specifically, MS, HCV infection, influenza infection and melanoma. Dosing is dependent on the severity of the symptoms and on the responsiveness of the subject to the active drug. Medically trained professionals can easily determine the optimum dosage, dosing methodology and repetition rates. In any case, the attending physician, taking into consideration the age, sex, weight and state of the disease of the subject to be treated, as well as other clinical parameters according to the invention, will determine the dose.

The invention further provides method and compositions for treating, preventing, ameliorating or delaying the onset of an immune-related disorder in a subject treated with interferon in a subject in need thereof. The composition of the invention comprises as an active ingredient a therapeutically effective amount of any one of: (a) antisense specific for miR-146a; (b) siRNA specific for miR-146a; and (c) miR-146a oligonucleotide. It should be noted that according to certain embodiments, the compound may either increase or decrease miR-146a expression and at least one of miR-146a regulated genes expression or products thereof.

More specifically, the compositions containing of any one of: (a) antisense specific for miR-146a; (b) siRNA specific for miR-146a; and (c) miR-146a oligonucleotide or any compound that modulates its expression and levels of the present invention, or any combination, mixture or cocktail thereof can be administered for prophylactic and/or therapeutic treatments. In therapeutic application, compositions are administered to a patient already affected by an immune-related disorder in an amount sufficient to cure or at least partially arrest the condition and its complications, specifically, relapse or recurrence of the disease. An amount adequate to accomplish this is defined as a “therapeutically effective dose.” Amounts effective for this use will depend upon the severity of the condition and the general state of the patient. Single or multiple administrations on a daily, weekly or monthly schedule can be carried out with dose levels and pattern being selected by the treating physician.

The term “prophylaxis” refers to prevention or reduction the risk of occurrence of the biological or medical event that is sought to be prevented in a tissue, a system, animal or human by a researcher, veterinarian, medical doctor or other clinician, and the term “prophylactic ally effective amount” is intended to mean that amount of a pharmaceutical composition that will achieve this goal.

In prophylactic applications, compositions containing any one of: (a) antisense specific for miR-146a and (b) siRNA specific for miR-146a or any compound that modulates its expression and levels or any combination, mixture or cocktail thereof are administered to a patient who is at risk of developing the disease state to enhance the patient's resistance. Such an amount is defined to be a “prophylactic ally effective dose”. In this use, the precise amounts again depend upon the patient's state of health and general level of immunity, as well as other clinical parameters according to the invention.

As used herein, “disease”, “disorder”, “condition” and the like, as they relate to a subject's health, are used interchangeably and have meanings ascribed to each and all of such terms.

The present invention relates to the treatment of subjects, or patients, in need thereof. By “patient” or “subject in need” it is meant any organism who may be affected by the above-mentioned conditions, and to whom the treatment and diagnosis methods herein described is desired, including humans. More specifically, the composition of the invention is intended for mammals. By “mammalian subject” is meant any mammal for which the proposed therapy is desired, including human, equine, canine, and feline subjects, most specifically humans.

It should be noted that specifically in cases of non-human subjects, the method of the invention may be performed using administration via injection, drinking water, feed, spraying, oral gavages and directly into the digestive tract of subjects in need thereof. It should be further noted that particularly in case of human subject, administering of any one of: (a) antisense specific for miR-146a; (b) siRNA specific for miR-146a; and (c) miR-146a oligonucleotide or any compound that modulates its expression and levels to the patient includes both self-administration and administration to the patient by another person.

The term “treatment or prevention” refers to the complete range of therapeutically positive effects of administrating to a subject including inhibition, reduction of, alleviation of, and relief from, a condition known to be treated with interferon, for example an immune-related disorder as detailed herein. More specifically, treatment or prevention of relapse or recurrence of the disease includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying metabolic causes of symptoms. It should be appreciated that the terms “inhibition”, “moderation”, “reduction” or “attenuation” as referred to herein, relate to the retardation, restraining or reduction of a process by any one of about 1% to 99.9%, specifically, about 1% to about 5%, about 5% to 10%, about 10% to 15%, about 15% to 20%, about 20% to 25%, about 25% to 30%, about 30% to 35%, about 35% to 40%, about 40% to 45%, about 45% to 50%, about 50% to 55%, about 55% to 60%, about 60% to 65%, about 65% to 70%, about 75% to 80%, about 80% to 85% about 85% to 90%, about 90% to 95%, about 95% to 99%, or about 99% to 99.9%.

With regards to the above, it is to be understood that, where provided, percentage values such as, for example, 10%, 50%, 120%, 500%, etc., are interchangeable with “fold change” values, i.e., 0.1, 0.5, 1.2, 5, etc., respectively.

All scientific and technical terms used herein have meanings commonly used in the art unless otherwise specified. The definitions provided herein are to facilitate understanding of certain terms used frequently herein and are not meant to limit the scope of the present disclosure.

As used herein the term “about” refers to ±10% The terms “comprises”, “comprising”, “includes”, “including”, “having” and their conjugates mean “including but not limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

As used herein the term “about” refers to ±10%. The terms “comprises”, “comprising”, “includes”, “including”, “having” and their conjugates mean “including but not limited to”. This term encompasses the terms “consisting of” and “consisting essentially of”. The phrase “consisting essentially of” means that the composition or method may include additional ingredients and/or steps, but only if the additional ingredients and/or steps do not materially alter the basic and novel characteristics of the claimed composition or method. Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

The term “about” as used herein indicates values that may deviate up to 1 percent, more specifically 5 percent, more specifically 10 percent, more specifically 15 percent, and in some cases up to 20 percent higher or lower than the value referred to, the deviation range including integer values, and, if applicable, non-integer values as well, constituting a continuous range.

It must be noted that, as used in this specification and the appended claims, the singular forms “a”, “an” and “the” include plural referents unless the content clearly dictates otherwise.

### EXAMPLES

### Experimental Procedures

The expression levels of the genes of interest were obtained from publicly available data bases [http://www.ncbi.nlm.nih.gov/geo/] using the following Gene Expression Omnibus Accession Nos:

Gene Expression Omnibus Accession No. GSE26104 (described in Example 1) provides gene expression microarrays data obtained from peripheral blood mononuclear cells (PBMC) of eight Multiple Sclerosis (MS) patients before treatment (baseline) and at 3, 12 and 24 months after IFN-13 treatment with BETAFERON or REBIF (total of 32 samples).

Gene Expression Omnibus Accession No. GSE17846 (described in Example 2) provides miRNA profiling data from total blood of MS patients (n=20) and of donors without known affection (n=21).

Gene Expression Omnibus Accession No GSE19224 (described in Example 3) provides paired comparison of RNA expression in PBMC of the same group of fourteen MS patients while stable and while in relapse. Microarrays were used to measure mRNA expression in the peripheral blood of the MS patients during clinical relapse and while stable.

Gene Expression Omnibus Accession No GSE20994 (described in Example 4) provides analysis of complete miRNA repertoire from peripheral blood of melanoma cancer patients (n=35) and normal controls (n=22).

Gene Expression Omnibus Accession No GSE11190 (described in Example 5) corresponded to a total of 78 samples obtained from biopsies (before and after interferon treatment) that were analyzed using Affymetrix Human U133 Plus 2.0 Array.

Gene Expression Omnibus Accession No GSE17183 (described in Example 5) provides hepatic gene expression in liver biopsy from 30 patients before and one week after starting combination therapy with IFN+Rib. Hepatocytes and liver-infiltrating lymphocytes were obtained from 12 patients using laser capture micro dissection.

Gene Expression Omnibus Accession No GSE18816 (described in Example 6) provides gene expression profiles in primary human macrophages after influenza A virus infection. Peripheral-blood leucocytes were separated from buffy coats of three healthy blood donors and cells were differentiated for 14 days before use. Differentiated macrophages were infected with H1N1 and H5N1 at a multiplicity of infection (MOI) of two. Total RNA was extracted from cells after 1, 3, and 6 h post-infection, and gene expression profiling was performed using an Affymetrix Human Gene 1.0 ST microarray platform.

The data was downloaded from the each one of these selected Gene Expression Omnibus Accession and was analyzed using custom programs written in MATLAB. Specifically, after verifying normalization of data (such as RMA quantile on Affymetrix arrays) and averaging multiple probes per gene, MATLAB mattest is carried out with permutations to calculate pvals. In brief, mattest perform two-sample t-test to evaluate differential expression of genes from two experimental conditions or phenotypes. This is used for the next step to perform the matlab mavolcano routine for example by using responders and non responders gene average values.

### Example 1

### Signature Genes that can Predict Response to Interferon Treatment in Multiple Sclerosis (MS) Patients

The changes in gene expression levels in MS patients before and after treatment with interferon were analyzed using the data available in Gene Expression Omnibus Accession No. GSE26104. The information provided in GSE26104 and the subsequent analysis was described above.

FIG. 1 shows a representation of genes, each depicted by a different point, such that each point represents the ratio of the specific gene between its expression after treatment and its base line value. Each point corresponds to an average value of the ratio of the specific gene calculated for all the eight MS patients in the cohort of patients. Each gene (point) is assigned with a value along the X axis that corresponds to the regulation fold (either up regulation or down regulation) and with a value along the Y axis corresponding to the significant of the regulation. Thus, this analysis provides a quantitative indication for the dominating genes that are regulated in MS patients treated for 3 month with respect to a baseline level determined before initiation of treatment.

The results indicate that MS patients that were found responsive to interferon treatment showed a distribution of genes expression with a high number of genes showing an up regulated profile after treatment. Specifically, as shown in Table 1, the following genes were found to be up regulated by interferon treatment IFI44L, MX2, RSAD2, IFIT5, IFITM1, IFITM3, IRF7, ISG15, IF127, TRAF6, IF144, IFIT3, OASL, TRIM22, IFIT1, IRAK1 and IRAK2,

TABLE 1
Up regulated genes in responsive MS patients.

| Gene | | RefSeq | RefSeq |
|---|---|---|---|
| Symbol | Gene Title | Transcript ID | Protein ID |
| IFI44L | Interferon-induced | NM_006820 | NP_006811 |
| | protein 44-like¹ | (SEQ ID NO: 39) | (SEQ ID NO: 40) |
| MX2 | Myxovirus (influenza | NM_002463 | NP_002454 |
| | virus) resistance 2 | (SEQ ID NO: 41) | (SEQ ID NO: 42) |
| | (mouse) | | |
| RSAD2 | Radical S-adenosyl | NM_080657 | NP_542388 |
| | methionine domain | (SEQ ID NO: 43) | (SEQ ID NO: 44) |
| | containing 2 | | |
| IFIT5 | Interferon-induced | NM_012420 | NP_036552 |
| | protein with | (SEQ ID NO: 45) | (SEQ ID NO: 46) |
| | tetratricopeptide | | |
| | repeats 5 | | |
| IFITM1 | Interferon induced | NM_003641 | NP_003632 |
| | transmembrane | (SEQ ID NO: 47) | (SEQ ID NO: 48) |
| | protein 1 | | |
| IFITM3 | Interferon induced | NM_021034 | NP_066362 |
| | transmembrane | (SEQ ID NO: 49) | (SEQ ID NO: 50) |
| | protein 3 | | |
| IRF7 | Interferon regulatory | NM_001572 | NP_001563 |
| | factor 7 | (SEQ D NO: 51) | (SEQ ID NO: 52) |
| | | NM_004029 | NP_004020 |
| | | (SEQ ID NO: 53) | (SEQ ID NO: 54) |
| ISG15 | ISG15 ubiquitin-like | NM_005101 | NM_005101 |
| | modifier | (SEQ ID NO: 55) | (SEQ ID NO: 56) |
| IFI27 | Interferon alpha- | NM_001130080 | NP_001123552 |
| | inducible protein 27 | (SEQ ID NO: 57) | (SEQ ID NO: 59) |
| | | NM_005532 | NP_005523 |
| | | (SEQ ID NO: 58) | (SEQ ID NO: 60) |
| TRAF6 | TNF receptor- | NM_145803 | NP_665802 |
| | associated factor 6, E3 | (SEQ ID NO: 61) | (SEQ ID NO: 62) |
| | ubiquitin protein ligase | NM_004620 | NP_004611 |
| | | (SEQ ID NO: 63) | (SEQ ID NO: 64) |
| IFI44 | Interferon-induced | NM_006417 | NP_006408 |
| | protein 44 | (SEQ ID NO: 65) | (SEQ ID NO: 66) |
| IFIT3 | Interferon-induced | NM_001031683 | NP_001026853 |
| | protein with | (SEQ ID NO: 67) | (SEQ ID NO: 68) |
| | tetratricopeptide | NM_001549 | NP_001540 |
| | repeats 3 | (SEQ ID NO: 69) | (SEQ ID NO: 70) |
| OASL | 2′-5′-oligoadenylate | NM_003733 | NP_003724.1 |
| | synthetase-like | (SEQ ID NO: 71) | (SEQ ID NO: 72) |
| | | NM_198213 | NP_937856.1 |
| | | (SEQ ID NO: 73) | (SEQ ID NO: 74) |
| TRIM22 | Tripartite motif | NM_001199573 | NP_001186502 |
| | containing 22 | (DEQ ID NO: 75) | (SEQ ID NO: 76) |
| | | NM_006074 | NP_006065 |
| | | (SEQ ID NO: 77) | (SEQ ID NO: 78) |
| IFIT1 | Interferon-induced | NM_001548 | NP_001539 |
| | protein with | (SEQ IS NO: 79) | (SEQ ID NO: 80) |
| | tetratricopeptide | | |
| | repeats 1 | | |
| IRAK1 | Interleukin-1 receptor- | NM_001025242 | NP_001020413 |
| | associated kinase 1 | (SEQ ID NO: 81) | (SEQ ID NO: 82) |
| | | NM_001025243 | NP_001020414 |
| | | (SEQ ID NO: 83) | (SEQ ID NO: 84) |
| | | NM_001569 | NP_001560 |
| | | (SEQ ID NO: 85) | (SEQ ID NO: 86) |
| IRKA2 | Interleukin-1 receptor- | NM_001570 | NP_001561 |
| | associated kinase 2 | (SEQ ID NO: 87) | (SEQ ID NO: 88) |

In the non-responder MS patients, this up regulation in the gene expression was not observed.

These results demonstrate the feasibility of using the expression level of this arsenal of genes (at least a predetermined group thereof) as a specific genetic biomarker to predict the response to interferon treatment. As the prediction can be obtained after a short treatment period, for example 3 month of treatment, those patients that do not show this genetic profile are considered to have a low probability to respond to further treatment. Additional unnecessary treatment can be thus avoided.

In addition, the inventors have found that some of the genes that were up regulated after treatment as compared to base line levels (as shown in FIG. 1) correspond to the genes previously found by Cameron et al., 2008 to be suppressed in miR-146a-expressing Akata cells.

Table 2 shows the expression of the miR-146a-controlled genes after three month treatment in each one of the MS patients separately (relative to a base line value).

TABLE 2
Change in gene expression of MS patients
after 3 month treatment with interferon.

| | Gene expression in MS patients (expression data of these | | | | | | | |
|---|---|---|---|---|---|---|---|---|
| Gene | genes was obtained from GSE26104) | | | | | | | |
| symbol | #1 | #2 | #3 | #4 | #5 | #6 | #7 | #8 |
| FI44L | 4.11 | 6.01 | 1.77 | 3.27 | 2.61 | 5.56 | 4.36 | 4.25 |
| IFI44 | 1.81 | 2.87 | 0.76 | 2.22 | 1.41 | 3.35 | 3.09 | 3.01 |
| MX2 | 1.14 | 1.92 | 0.73 | 2.54 | 0.91 | 2.52 | 2.28 | 2.52 |
| RSAD2 | 2.8 | 4.45 | 1.05 | 4.58 | 3.56 | 4.75 | 5.72 | 4.97 |
| IFIT3 | 1.84 | 0.69 | 1.04 | 4.17 | 2.44 | 3.43 | 4.55 | 2.19 |
| OASL | 1.15 | 2.08 | 0.7 | 2.83 | 2.18 | 3.6 | 4.19 | 3.68 |
| TRIM22 | 0.86 | 0.98 | 0.26 | 1.14 | 0.9 | 0.66 | 1.35 | 1.27 |
| IFIT1 | 2.06 | 1.57 | 1.11 | 4.59 | 2.72 | 3.98 | 4.89 | 2.74 |
| IFIT5 | 0.71 | 0.02 | 0.82 | 1.14 | 1.76 | 1.71 | 2.22 | 1.56 |
| IFITM1 | 0.64 | 1.16 | 0.13 | 1.25 | 1.75 | 1.06 | 2.39 | 1.66 |
| IFITM3 | 1.01 | 1.77 | 1.14 | 2.1 | 1.17 | 1.91 | 2.6 | 1.64 |
| IRF7 | 0.56 | 1.31 | 0.63 | 2.27 | 1.41 | 2.24 | 2.1 | 1.88 |
| ISG15 | 1.24 | 2.78 | 0.28 | 3.08 | 2.22 | 3.46 | 3.52 | 3.06 |
| IFI27 | 5.38 | 7.16 | 1.54 | 5.96 | 7.98 | 7.27 | 8.35 | 6.8 |

As shown in Table 2, patient #3 shows a different gene distribution pattern that does not include up regulation of most these genes. Without being bound by any theory, it can be assumed that the genes were not up regulated in patient #3 since there is a high expression of miR-146a gene that interferes with this up regulation and lead to non responsive.

Based on these results, the inventors have concluded that the miR-146a-controlled genes are being up-regulated in MS patients after 3, 12 and 24 months of interferon treatment.

Patients diagnosed with high level of miR-146a are most likely to have a genetic predisposition of interferon resistance. Thus, the miR-146a gene can be considered a proportional negative attenuator of the interferon response genes.

### Example 2

### miR-146a Expression in Healthy and MS Patients

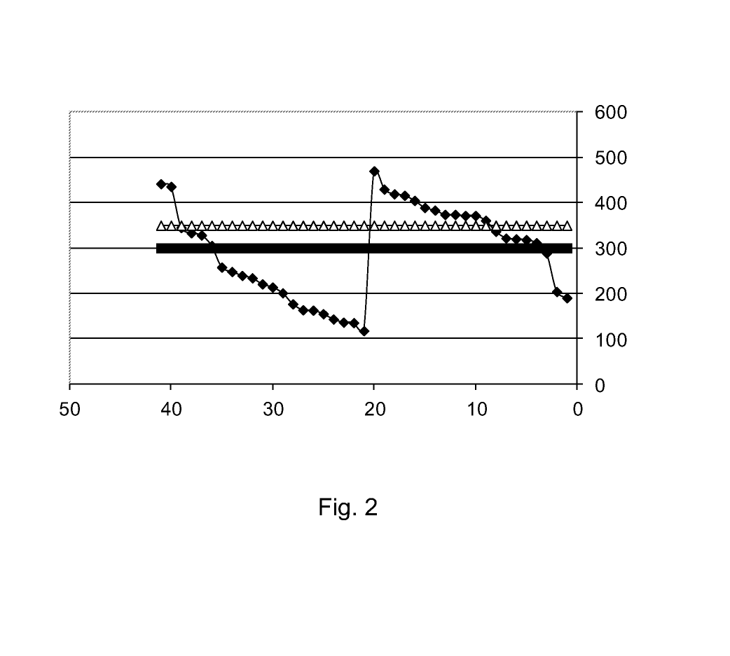

Expression profile of miR-146 in MS patients was obtained from GSE17846. The information provided in GSE17846 and the data analyses were described above. The normalized values of the expression level of the miR-146a gene that were computed using the freely available R software are presented by FIG. 2.

As shown in FIG. 2, there is a difference in the overall expression level of miR-146a in MS patients and healthy donors with the expression level in the healthy donors (subjects 21 to 41) being lower than the level in the MS patients (subjects 1 to 20).

By sorting the values of both MS and healthy miR-146a expression and quantitatively comparing the values of the patients, in comparison to normal healthy controls, a diagnostic predictor can be developed providing means for avoiding a non-response to interferon treatment for MS patients.

On the left hand side of FIG. 2, almost all healthy controls have an expression level lower than 350 (which are normalized read out values from the miR microarray). On the right hand side of FIG. 2, almost all the MS patients have expression values above this value (approx. 12) and are assumed to have a level of miR-146a that will not enable up regulation of IFN responsive genes, turning the patient to a non responder.

Thus, the data shown here can provide a diagnostic marker for identifying MS patients that will not be responsive to interferon treatment based on the normalized expression level of miR-146a. It can be also assumed that in order to avoid non responsiveness of patients, the expression level of miR-146a should be down regulated and thus turning the patients to a responsive genetic profile. There are several methods known in the art for down regulation of miR-146a described for example in US2007232553A, US2009203136, or treating the patient with other means.

### Example 3

### Signature Genes that can Predict Remission or Relapse in MS Patients

Multiple sclerosis is often characterized by the occurrence of clinical relapses separated by periods of clinical stability and thus identifying and understanding the events related to clinical relapse might be helpful in assessing the patient's condition and treatment requirements. To evaluate which genes can predict if MS patients treated with interferon will experience a stable condition or a relapse of the disease, data from GSE19224 was analyzed. The information provided in GSE192244 and the analysis was described above.

The graph shown in FIG. 3 is as explained in Example 1. The data shown in FIG. 3 depicts the ratio between the expression of a specific gene in the same patient during relapse vs. its expression when stable. Thus, the genes present in the left hand side of FIG. 3 having a negative log 2 value correspond to genes that are down regulated in a relapse period.

As can be seen in FIG. 3, some of the genes that are down regulated during relapse are interferon genes. Specifically, the following interferon genes were found to be down regulated by interferon treatment IFIT3, IFITM3, and IFIT2.

This down regulation observed during relapse can be explained by an over-expression of miR-146a. This analysis is in line with the results obtained in Example 1, which show that interferon genes are unregulated in responsive MS patients after interferon treatment and thus a down regulation in their expression level can predict that the patient is no longer in a responsive state and is thus genetically predisposed to relapse of the disease.

### Example 4

### miR-146a Expression in Melanoma Patients

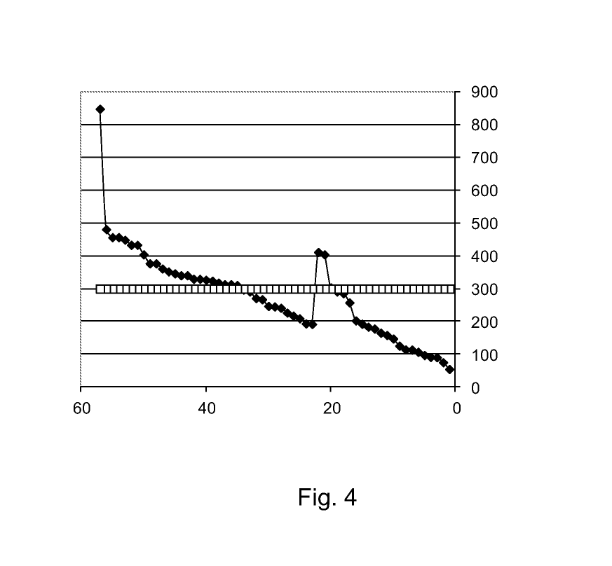

The role of miR-146a gene in multiple melanoma patients was evaluated, by using Expression data from GSE20994. The information obtained from GSE20994 and the analyses were described above. Normalized values of the expression level of the miR-146a gene that were computed by using the freely available R software are presented by FIG. 4.

As shown in FIG. 4, there is a difference in the overall expression level of miR-146a in melanoma patients and healthy volunteers. Specifically, the expression level of the miR-146a gene in the healthy donors (subjects 1 to 22) is somewhat lower than the level in the melanoma patients (subjects 23 to 57).

By sorting the values of both melanoma and healthy miR-146a expression and quantitatively comparing the values of the patients, in comparison to normal healthy controls, a diagnostic predictor of melanoma can be obtained. Moreover, the data shown here can provide a diagnostic marker for identifying melanoma patients.

Specifically, on the right hand side of FIG. 4, almost all healthy controls are at the level below the line at number 300. On the left hand side of FIG. 4, most of the melanoma patients have an expression level that is above the yellow line (nos. 35-57 have a miR-146A expression level of 300 or more). These melanoma patients are assumed to have a level of miR-146a that will not enable up regulation of interferon, making the patient a non responder that will not enable up regulation of interferon.

Thus, the results shown here serve as a diagnostic marker and can be used for example by measuring the miR-146a level before or during the treatment. A level above a normalized value of 300 obtained from a miR-array predicts a patient to be considered a non responder to interferon treatment. In addition, the higher the expression level, the possibility for a person to respond decrease. It can be also assumed that in order to avoid non responsiveness of patients, the expression level of miR-146a may be down regulated using any method described in Example 2 above.

### Example 5

### Genes Associated with Interferon Treatment IN Hepatitis C Patients

This example was aimed to evaluate the changes in the expression level of genes controlled by miR-146a in patients diagnosed with Hepatitis C virus (HCV), measured in tissue extracted one week before and one week after interferon treatment.

The information obtained from GSE11190 and GSE17183 and the analyses were described above.

FIG. 5 shows the gene expression pattern obtained one week after treatment that includes an up regulation pattern in a variety of genes, some of which are associated with interferon. As shown by the Figure, a clear up-regulation of miR-146a genes was demonstrated for responder patients.

International Patent Application WO10076788, that is a previous application by the inventor, describes five signature genes that are up regulated in patients that are considered non-responders to interferon treatment. Thus, based on the expression of the five signature genes before treatment, one can assess the probability to respond to treatment. In addition, four hours following an interferon treatment, these five signature genes were not up regulated in non-responders (as their initial expression value was higher before treatment). In the non-responders patients no up regulation of genes were observed after treatment.

Thus, for non-responders HCV patients, an up-regulation of miR-146a can be assumed. Accordingly, hepatic C virus may be treated by determining the patients that are considered non-responders, namely having a high miR-146a expression and providing them a treatment to reduce this expression as described inheres above in Example 2. Thereafter the interferon treatment would be expected to be more effective as it will be effective in patients originally considered as non-responders.

Performing receiver operating characteristic (ROC) curve assessment on the previous Canadian microarray dataset (Chen (2005); Dill (2011) and Onomoto 1 (2011) and additional similar sets reveals not much ROC curve area changes when adding more genes from the signature genes meaning they all operate correlated and in synchrony, which strengthen the potential role of one key ruler such as the miR-146a.

### Example 6

### Genes Associated with Influenza Virus Infection

This example was aimed to evaluate the changes in the expression level of genes following viral infections. The information obtained from GSE18816 and the analysis was described above.

FIGS. 6A and 6B show the distribution of the gene expression as measured one hour, and six hours, respectively post-infection with H5N1 virus in vitro. FIG. 6C shows the distribution of the gene expression as measured six hours, post-infection with H1N1 virus in vitro.

The results show that one hour post infection, none of the tested gene is up regulated or down regulated by more than two fold compared to control (FIG. 6A). However, six hours post infection with H5N1 (FIG. 6B) a pattern of up regulation in different genes is observed. In addition, a large number of genes are up regulated after six hours in the H1N1 infected cells (FIG. 6C) compared with the H5N1 infected cells after 6 hours (FIG. 6B).

These results provide insight into the host response to H5N1 and H1N1 infections and provide diagnostic means to identify infections.

Accordingly, when a host is infected with H5N1 or H1N1 virus, endogenous interferon is being secreted leading to an up regulation of interferon related genes (as seen in FIGS. 6B and 6C). This indicates that the host is responding to interferon and thus can be treated with additional amounts of exogenous interferon.

Without being bound by theory, it may be assumed that an up regulation of these genes in response to a viral infection indicates that the immune response in the host being infected by the virus has produced endogenous interferon that in turn led to up regulation of the genes. Such a host may be considered responder to interferon treatment.

Without being bound by theory, it may also be assumed that an up regulation of these genes is associated with a low expression level of miR146a that enables the up regulation of the genes.

As can be seen in FIGS. 6B and 6C, the up regulated genes are miR-146a controlled genes. Thus, affecting miR-146a level provides a potential route to battle the virus.

The examples herein thus show, that the expression level of miR-146A and/or a miR-146A regulated gene in a patient suffering from a disease may be used to define whether an additional treatment, should be provided to that patient, prior to an interferon treatment, to make the interferon treatment more effective in that particular patient.

TABLE 3
List of Sequences

| SEQ | |
|---|---|
| ID NO: | Details |
| 1 | RNA sequence of mature miR-146a |
| 2 | RNA sequence of pre-miR-146a |
| 3 | cDNA of mature miR-146a |
| 4 | cDNA of pre-miR-146a |
| 5 | DNA of miR-146a primary transcript |
| 6 | DNA of miR-146a primary transcript |
| 7 | DNA sequence of interferon alpha 1 |
| 8 | Protein sequence interferon alpha 1 |
| 9 | DNA sequence of interferon alpha 2 |
| 10 | Protein sequence of interferon alpha 2 |
| 11 | DNA sequence of Interferon alpha-4 |
| 12 | Protein sequence of Interferon alpha-4 |
| 13 | DNA sequence of Interferon alpha-5 |
| 14 | Protein sequence of Interferon alpha-5 |
| 15 | DNA sequence of Interferon alpha-6 |
| 16 | Protein sequence of Interferon alpha-6 |
| 17 | DNA sequence of Interferon alpha-7 |
| 18 | Protein sequence of Interferon alpha-7 |
| 19 | DNA sequence of Interferon alpha-8 |
| 20 | Protein sequence of Interferon alpha-8 |
| 21 | DNA sequence of Interferon alpha-10 |
| 22 | Protein sequence of Interferon alpha-10 |
| 23 | DNA sequence of Interferon alpha-1/13 |
| 24 | Protein sequence of Interferon alpha-1/13 |
| 25 | DNA sequence of Interferon alpha-14 |
| 26 | Protein sequence of Interferon alpha-14 |
| 27 | DNA sequence of Interferon alpha-16 |
| 28 | Protein sequence of Interferon alpha-16 |
| 29 | DNA sequence of Interferon alpha-17 |
| 30 | Protein sequence of Interferon alpha-17 |
| 31 | DNA sequence of Interferon alpha-21 |
| 32 | Protein sequence of Interferon alpha-21 |
| 33 | DNA sequence of Interferon, beta 1 |
| 34 | Protein sequence of Interferon, beta 1 |
| 35 | DNA sequence of Interferon omega-1 |
| 36 | Protein sequence of Interferon omega-1 |
| 37 | DNA sequence of Interferon-gamma |
| 38 | Protein sequence of Interferon-gamma |
| 39 | DNA sequence of Interferon-induced protein 44-like (IFI44L) |
| 40 | Protein sequence of Interferon-induced protein 44-like (IFI44L) |
| 41 | DNA sequence of Myxovirus (influenza virus) resistance 2 |
| | (MX2) |
| 42 | Protein sequence of Myxovirus (influenza virus) resistance 2 |
| | (MX2) |
| 43 | DNA sequence of Radical S-adenosyl methionine domain |
| | containing 2 (RSAD2) |
| 44 | Protein sequence of Radical S-adenosyl methionine domain |
| | containing 2 (RSAD2) |
| 45 | DNA sequence of Interferon-induced protein with tetratrico- |
| | peptide repeats 5 (IFIT5) |
| 46 | Protein sequence of Interferon-induced protein with tetratrico- |
| | peptide repeats 5 (IFIT5) |
| 47 | DNA sequence of Interferon induced transmembrane protein 1 |
| | (IFITM1) |
| 48 | Protein sequence of Interferon induced transmembrane protein 1 |
| | (IFITM1) |
| 49 | DNA sequence of Interferon induced transmembrane protein 3 |
| | (IFITM3) |
| 50 | Protein sequence of Interferon induced transmembrane protein 3 |
| | (IFITM3) |
| 51 | DNA sequence of Interferon regulatory factor 7 (IRF7) |
| 52 | Protein sequence of Interferon regulatory factor 7 (IRF7) |
| 53 | DNA sequence of Interferon regulatory factor 7 (IRF7) |
| 54 | Protein sequence of Interferon regulatory factor 7 (IRF7) |
| 55 | DNA sequence of ISG15 ubiquitin-like modifier (ISG15) |
| 56 | Protein sequence of ISG15 ubiquitin-like modifier (ISG15) |
| 57 | DNA sequence of Interferon alpha-inducible protein 27 (IFI27) |
| 58 | protein sequence of Interferon alpha-inducible protein 27 (IFI27) |
| 59 | DNA sequence of Interferon alpha-inducible protein 27 (IFI27) |
| 60 | Protein sequence of Interferon alpha-inducible protein 27 (IFI27) |
| 61 | DNA sequence of TNF receptor-associated factor 6, E3 ubiquitin |
| | protein ligase (TRAF6) |
| 62 | Protein sequence of TNF receptor-associated factor 6, E3 |
| | ubiquitin protein ligase (TRAF6)e |
| 63 | DNA sequence of TNF receptor-associated factor 6, E3 ubiquitin |
| | protein ligase (TRAF6) |
| 64 | protein sequence of TNF receptor-associated factor 6, E3 |
| | ubiquitin protein ligase (TRAF6) |
| 65 | DNA sequence of Interferon-induced protein 44 (IFI44) |
| 66 | Protein sequence of Interferon-induced protein 44 (IFI44) |
| 67 | DNA sequence of Interferon-induced protein with tetratrico- |
| | peptide repeats 3 (IFIT3) |
| 68 | Protein sequence of Interferon-induced protein with tetratrico- |
| | peptide repeats 3 (IFIT3) |
| 69 | DNA sequence of Interferon-induced protein with tetratrico- |
| | peptide repeats 3 (IFIT3) |
| 70 | Protein sequence of Interferon-induced protein with tetratrico- |
| | peptide repeats 3 (IFIT3) |
| 71 | DNA sequence of 2′-5′-oligoadenylate synthetase-like (OASL) |
| 72 | Protein sequence of 2′-5′-oligoadenylate synthetase-like (OASL) |
| 73 | DNA sequence of 2′-5′-oligoadenylate synthetase-like (OASL) |
| 74 | Protein sequence of 2′-5′-oligoadenylate synthetase-like (OASL) |
| 75 | DNA sequence of Tripartite motif containing 22 (TRIM22) |
| 76 | Protein sequence of Tripartite motif containing 22 (TRIM22) |
| 77 | DNA sequence of Tripartite motif containing 22 (TRIM22) |
| 78 | Protein sequence of Tripartite motif containing 22 (TRIM22) |
| 79 | DNA sequence of Interferon-induced protein with tetratrico- |
| | peptide repeats 1 (IFIT1) |
| 80 | Protein sequence of Interferon-induced protein with tetratrico- |
| | peptide repeats 1 (IFIT1) |
| 81 | DNA sequence of Interleukin-1 receptor-associated kinase 1 |
| | (IRAK1) |
| 82 | Protein sequence of Interleukin-1 receptor-associated kinase 1 |
| | (IRAK1) |
| 83 | DNA sequence of Interleukin-1 receptor-associated kinase 1 |
| | (IRAK1) |
| 84 | Protein sequence of Interleukin-1 receptor-associated kinase 1 |
| | (IRAK1) |
| 85 | DNA sequence of Interleukin-1 receptor-associated kinase 1 |
| | (IRAK1) |
| 86 | Protein sequence of Interleukin-1 receptor-associated kinase 1 |
| | (IRAK1) |
| 87 | DNA sequence of Interleukin-1 receptor-associated kinase 2 |
| | (IRAK2) |
| 88 | Protein sequence of Interleukin-1 receptor-associated kinase 2 |
| | (IRAK2) |
| 89 | RNA sequence of anti-sense miR-146a (Artificial Sequence) |
| 90 | Probe sequence for IRAK2 |
| 91 | Probe sequence for IRAK2 |
| 92 | DNA sequence of probe for mature miR-146a(Artificial |
| | Sequence) |
| 93 | 5′-primer for miR-146a |
| 94 | 3′-primer for miR-146a |
| 95 | 5′-primer for miR-146a primary transcript |
| 96 | 5′-primer for miR-146a primary transcript |
| 97 | 5′-primer for miR-146a primary transcript |
| 98 | 5′-primer for miR-146a primary transcript |
| 99 | 3′-primer for miR-146a primary transcript |
| 100 | 3′-primer for miR-146a primary transcript |
| 101 | 3′-primer for miR-146a primary transcript |
| 102 | 3′-primer for miR-146a primary transcript |
| 103 | Probe sequence for IFI44L |
| 104 | Probe sequence for MX2 |
| 105 | Probe sequence for RSAD2 |
| 106 | Probe sequence for IFIT5 |
| 107 | Probe sequence for IFITM1 |
| 108 | Probe sequence for IFITM1 |
| 109 | Probe sequence for IFITM3 |
| 110 | Probe sequence for IRF7 |
| 111 | Probe sequence for ISG15 |
| 112 | Probe sequence for IFI27 |
| 113 | Probe sequence for TRAF6 |
| 114 | Probe sequence for IFI44 |
| 115 | Probe sequence for IFIT3 |
| 116 | Probe sequence for OASL |
| 117 | Probe sequence for OASL |
| 118 | Probe sequence for TRIM22 |
| 119 | Probe sequence for IFIT1 |
| 120 | Probe sequence for IRAK1 |
| 121 | Probe sequence for IRAK1 |

### LIST OF PUBLICATIONS

Chen Limin, et al., Gastroenterology 128:1437-1444 (2005).
Taylor, M W, et al., Journal of Virology 81:3391-3401 (2007).
van Baarsen L G, et al., PLoS ONE 3:e1927 (2008).
Zeremski M, et al., J. Acquir. Immune Defic. Syndr. 45:262-268 (2007).
Tarantino G, et al., Digestive and Liver Disease 40:A1-A40 (2008).
US2009/157324
WO10/076788
Williams A E, Cell Mol Life Sci. 65:545-562 (2008).
Taganov K D, et al., Proc. Natl. Acad. Sci. USA. 103:12481-12486 (2006).
U.S. Pat. No. 6,258,569
U.S. Pat. No. 6,030,787
U.S. Pat. No. 5,952,202
U.S. Pat. No. 5,876,930
U.S. Pat. No. 5,866,336
U.S. Pat. No. 5,736,333
U.S. Pat. No. 5,723,591
U.S. Pat. No. 5,691,146
U.S. Pat. No. 5,538,848
Harlow and Lane, Antibodies: A Laboratory Manual, Cold Spring Harbor Laboratory, New York, 1988.
Witebsky E, et al., J. Am. Med. Assoc. 164: 1439-47 (1957).
Jazdzewski K, et al., Proc. Natl. Acad. Sci. USA. 105:7269-74 (2008).
Cameron J E, et al., Journal of Virology 82:1946-1958 (2008).
US2007/232553
US2009/203136
Limin Chen et al., Gastroenterology 128:1437-1444 (2005)
Michael T. Dill, et al., Gastroenterology 140:1021-1031 (2011)
Koji Onomotol et al., Plos one 6 (5):19799 (2011)

1. A prognostic method for predicting, assessing and monitoring responsiveness of a mammalian subject to interferon treatment, said method comprising the steps of:
(a) determining the level of expression of miR-146a and optionally of at least one of miR-146a regulated genes in a biological sample of said subject to obtain an expression value;
(b) comparing the expression value obtained in step (a) to a predetermined standard expression value or to an expression value of miR146a and optionally of at least one of miR-146a regulated genes in at least one control sample; thereby predicting, assessing and monitoring responsiveness of a mammalian subject to interferon treatment

2. The method according to claim 1, for predicting responsiveness of a mammalian subject to interferon treatment, said method comprising the steps of:
(a) determining the level of expression of miR-146a and optionally of at least one of miR-146a regulated genes in at least one biological sample of said subject to obtain an expression value;
(b) comparing the expression value obtained in step (a) to a predetermined standard expression value or to an expression value of miR146a and optionally of at least one of miR-146a regulated genes in a control sample;
wherein a positive expression value (OR a higher expression value) of said miR146a and optionally of at least one of miR-146a regulated genes as compared to said predetermined standard expression value or optionally, to said expression value of at least one control sample, indicates that said subject belongs to a pre-established population associated with lack of responsiveness to interferon treatment, thereby predicting responsiveness of a mammalian subject to interferon treatment.

3. The method according to claim 1, for assessing responsiveness of a mammalian subject to interferon treatment or evaluating the efficacy of interferon treatment on a subject, said method comprises the step of:
(a) determining the level of expression of at least one of miR-146a and of at least one of miR-146a regulated genes in a biological sample of said subject to obtain an expression value, wherein said sample is obtained prior to initiation of said treatment;
(b) determining the level of expression of at least one of miR-146a and of at least one of miR-146a regulated genes in at least one other biological sample of said subject, to obtain an expression value in said sample, wherein said at least one other sample is obtained after initiation of said treatment;
(c) calculating the rate of change between the expression value obtained in step (a), and the expression value obtained in step (b);
(d) comparing the rate of change obtained in step (c) with a predetermined standard rate of change determined between at least one sample obtained prior to and at least one sample obtained following interferon treatment, or to the rate of change calculated for expression values in at least one control sample obtained prior and following interferon treatment;
wherein at least one of a negative or equal rate of change of miR-146a expression value and a positive rate of change in the expression values of at least one of miR-146a regulated genes in said sample as compared to a predetermined standard rate of change or to the rate of change calculated for expression values in at least one control sample obtained prior and following interferon treatment, indicates that said subject belongs to a pre-established population associated with responsiveness to interferon treatment, thereby assessing responsiveness of a mammalian subject to interferon treatment or evaluating the efficacy of interferon treatment on said subject.

4. The method according to claim 1, for monitoring disease progression or early prognosis for disease relapse, said method comprises the steps of:
(a) determining the level of expression of miR-146a and optionally of at least one of miR-146a regulated genes in a biological sample of said subject to obtain an expression value;
(b) repeating step (a) to obtain expression values of at least one of miR-146a and of at least one of miR-146a regulated genes, for at least one more temporally-separated test sample;
(c) calculating the rate of change of said expression values of at least one of miR-146a and of at least one of miR-146a regulated genes between said temporally-separated test samples;
(d) comparing the rate of change obtained in step (c) with a predetermined standard rate of change determined for expression value between samples obtained from at least one subject in remission and in relapse following interferon treatment or to the rate of change calculated for expression values in at least one control sample obtained in remission and in relapse following interferon treatment;
wherein at least one of a positive rate of change of miR-146a expression value and a negative rate of change in the expression values of at least one of miR-146a regulated genes in said sample as compared to a predetermined standard rate of change or to the rate of change calculated for expression values in said at least one control sample, indicates that said subject belongs to a pre-established population associated with relapse, thereby monitoring disease progression or providing an early prognosis for disease relapse.

5. The method according to claim 1, wherein determining the level of expression of miR-146a and optionally of at least one of miR-146a regulated genes in a biological sample of said subject is performed by the step of contacting detecting molecules specific for miR-146a and optionally for at least one of miR-146a regulated genes with a biological sample of said subject, or with any nucleic acid or protein product obtained therefrom, wherein said detecting molecules are selected from isolated detecting nucleic acid molecules and isolated detecting amino acid molecules, said nucleic acid detecting molecules comprise isolated oligonucleotides, each oligonucleotide specifically hybridizes to a nucleic acid sequence of miR-146a or of one of said at least one of miR-146a regulated genes and optionally, to a control miRNA or control reference gene and wherein said detecting molecule is at least one of a pair of primers or nucleotide probes.

6. The method according to claim 1, wherein said miR-146a regulated genes are selected from a group consisting of IFI44L, MX2, RSAD2, IFIT5, IFITM1, IFITM3, IRF7, ISG15, IF127, TRAF6, IF144, IFIT3, OASL, TRIM22, IFIT1, IRAK1 and IRAK2.

7-9. (canceled)

10. The method according to claim 1, wherein said sample is any one of peripheral blood mononuclear cells and biopsies of organs or tissues.

11. The method according to claim 1, wherein said subject is suffering from an immune-related disorder, said immune-related disorder is any one of autoimmune disease, an infectious condition and a proliferative disorder.

12. (canceled)

13. The method according to claim 11, wherein said subject is suffering from Multiple sclerosis (MS).

14. The method according to claim 11, wherein said subject is suffering from an infectious condition selected from HCV or influenza infection.

15. The method according to claim 11, wherein said subject is suffering from melanoma.

16. The method according to claim 1, wherein determining the level of expression of miR-146a further comprises detecting the presence of a single-nucleotide polymorphism (SNP) in at least one of immature or mature miR-146a.

17. A prognostic composition comprising:
(a) detecting molecules specific for determining the level of expression of miR-146a in a biological sample; and
(b) detecting molecules specific for determining the level of expression of at least one of miR-146a regulated genes in a biological sample;
optionally, said detecting molecules of (a) and (b) are attached to a solid support,
wherein said composition is for predicting, assessing and monitoring responsiveness of a mammalian subject to interferon treatment.

18. (canceled)

19. A kit comprising:
(a) detecting molecules specific for determining the level of expression of miR-146a in a biological sample;
(b) detecting molecules specific for determining the level of expression of at least one of miR-146a regulated genes in a biological sample; and optionally at least one of:
(c) pre-determined calibration curve providing standard expression values of at least one of miR-146a and of at least one of miR-146a regulated genes;
(d) at least one control sample.

20. The kit according to claim 19, wherein said kit is a prognostic kit for predicting, assessing and monitoring responsiveness of a mammalian subject to interferon treatment.

21. The kit according to claim 20, further comprising instructions for use, wherein the instructions comprises at least one of:
(a) instructions for carrying out the detection and quantification of expression of said at least one of miR-146a or said at least one miR-146a regulated gene and optionally, of the control reference miRNA or a control reference gene; and
(b) instructions for comparing the expression values of at least one of said miR-146a and at least one of miR-146a regulated genes with a corresponding predetermined standard expression value.

22. The kit according to claim 19, wherein said miR-146a regulated genes are selected from a group consisting of IFI44L, MX2, RSAD2, IFIT5, IFITM1, IFITM3, IRF7, ISG15, IF127, TRAF6, IF144, IFIT3, OASL, TRIM22, IFIT1, IRAK1 and IRAK2.

23. The kit according to claim 19, wherein said detecting molecules are selected from isolated detecting nucleic acid molecules and isolated detecting amino acid molecules, wherein said nucleic acid detecting molecules comprises isolated oligonucleotides, each oligonucleotide specifically hybridize to a nucleic acid sequence of miR-146a or of one of said at least one of miR-146a regulated genes and optionally, to a control miRNA or control reference gene, said detecting molecule is at least one of a pair of primers or nucleotide probes.

24-25. (canceled)

26. The kit according to claim 19, further comprising at least one reagent for conducting a nucleic acid amplification based assay selected from the group consisting of a Real-Time PCR, micro arrays, PCR, in situ Hybridization and Comparative Genomic Hybridization.

27. A method for treating, preventing, ameliorating or delaying the onset of an immune-related disorder in a subject, said method comprises:
(a) predicting, assessing and monitoring responsiveness of said subject to interferon treatment according to the method of claim 1; and
(b) selecting an interferon treatment regimen based on said responsiveness thereby treating said subject.

28. (canceled)


SEQUENCE LISTING (121 sequences)

SEQ ID NO: 1 - RNA, 22 nt, Homo sapiens
ugagaacugaauuccauggguu

SEQ ID NO: 2 - RNA, 99 nt, Homo sapiens
ccgauguguauccucagcuuugagaacugaauuccauggguugugucagugucagaccucugaaauucaguucuucagcugggauaucucugucaucgu

SEQ ID NO: 3 - DNA, 22 bp, Homo sapiens
tgagaactgaattccatgggtt

SEQ ID NO: 4 - DNA, 99 bp, Homo sapiens
ccgatgtgtatcctcagctttgagaactgaattccatgggttgtgtcagtgtcagacctctgaaattcagttcttcagctgggatatctctgtcatcgt

SEQ ID NO: 5 - DNA, 2329 bp, Homo sapiens
gaaagatgctctttctccaagacgcttgaccgctcttcctttcctggatggcaccagcagggccgattggagtggtaaaccctgggccggaaggcatgccaaagggtggacaggatggacaggagacagtagcacaacgaggagggggagaacagcggctgaattggaaatgataaaataaaatgaaattttaggagctcgctggctgggacaggcctggactgcaaggaggggtctttgcaccatctctgaaaagccgatgtgtatcctcagctttgagaactgaattccatgggttgtgtcagtgtcagacctctgaaattcagttcttcagctgggatatctctgtcatcgtgggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattacttacgatgattcaggtactgttgtaagtgctttacatgctgttatacgagactcttgggagaaatcactttaatgaagcttgagacacatggcattgccatgcaatgatttttcccccctcttcacgggatcagagggaactaatagaatgtgacaatgattctttagcagggactgctgaggcttctggttcctttttaagatctgcagtgaaagaagatgagaaacatggatatgcccttcttttggtccccctcttcctttatttgatctctacttccttctataaatatattagggctacattgtccctttgtatttcaaacaaggcaaaaagaggttgtaattacactttactgcaatcctcagtttctccagggaacaggaatgcaaaggctttgaaggcctctctatttgctgacatggtcagctgggtgccatgggccaagtccttctgttgccctcctctgtcaccaagtaagctaggtcctttctgaggctcaggtttgctgtgatgatgatcacttttaggcagaaggttagaggcctcatgagtgctatatggactttattaggctttagatttgatggggaataagggatgtgatttgtcttttgggaactcatctttgattcatcattgtctcttggtatcttggaatttccatgtcattacagtctacagaatgaaagagtaacctgtcccagaggagaggcaggtgaaagactccacagcatgctcattctcattctgtcttctcagtgacaccgaggtttactgagtgcccactatgtgccaagcactgtgctcagggctttctttgtatgcatgatctcagtgaatctcaccaagcctcatctggaaaacggggacaaattaacaacaggatggcaaattgaaaaacacgtaaccatgttctacagatggaaaggggtgcttggttattatgaaggccccctcgcaagcgtgtgggacatgggtgtgttctctgggttgtactgatcagatcaaggacctcccccacccttctcacactctgcccacttccgccctttgcttatcagacccttagccagtgactcattccagaaccagaaccttggtgaaatctcaaccgacaccagagatcggtgtcttcagtcctagactgatggagaaaatccagaatatatactagaagctccaaatgctctgggtttcagctcctctgtgctgtggacactgactttggctcagaactccgatttagtacaaaaggctcatttttatttcaggggcactcttcctaaagcaaacctaataaatgaaatatggaattcacagatacacacacacattaaaaaattaacctagtgtatctgtgaggagtaggcagaaattcactgtataaaagaatgcttcatttcatagagaatttgtgttaagattccattagatagtacatttctcaaagatttttgaggttgtatttgctttaccaaaacttggtttatgtaagtggaaaaagcatgttgcaaaataacttggtgtctatgattcagtttatgtaaaataataaatgtatgtaggaatacgtgtgttgaaagatgtacatcaatttgctaacaatggttatctctgacgtggtgggatttgagatgtgtttttctttttggttgtatttttctctattgtttgacttaacacagaacatgtttggttacaacaataaagttattgaagac

SEQ ID NO: 6 - DNA, 2337 bp, Homo sapiens
tctccaagacgcttgaccgctcttcctttcctggatggcaccagcagggccgattggagtggtaaaccctgggccggaaggcatgccaaagggtggacaggatggacaggagacagtagcacaacgaggagggggagaacagtggctgaattggaaatgataaaataaaatgaaattttaggagctcgctggctgggacaggcctggactgcaaggaggggtctttgcaccatctctgaaaagccgatgtgtatcctcagctttgagaactgaattccatgggttgtgtcagtgtcagacctgtgaaattcagttcttcagctgggatatctctgtcatcgtgggcttgaggacctggagagagtagatcctgaagaactttttcagtctgctgaagagcttggaagactggagacagaaggcagagtctcaggctctgaaggtataaggagtgtgagttcctgtgagaaacactcatttgattgtgaaaagacttgaattctatgctaagcagggttccaagtagctaaatgaatgatctcagcaagtctctcttgctgctgctgctactcgtttacatttattgattacttacgatgattcaggtactgttgtaagtgctttacatgctgttatacgagactcttgggagaaatcactttaatgaagcttgagacacatggcattgccatgcaatgatttttcccccctcttcacgggatcagagggaactaatagaatgtgacaatgattctttagcagggactgctgaggcttctggttcctttttaagatctgcagtgaaagaagatgagaaacatggatatgcccttcttttggtccccctcttcctttatttgatctctacttccttctataaatatattagggctacattgtccctttgtatttcaaacaaggcaaaaagaggttgtaattacactttactgcaatcctcagtttctccagggaacaggaatgcaaaggctttgaaggcctctctatttgctgacatggtcagctgggtgccatgggccaagtccttctgttgccctcctctgtcaccaagtaagctaggtcctttctgaggctcaggtttgctgtgatgatgatcacttttaggcagaaggttagaggcctcatgagtgctatatggactttattaggctttagatttgatggggaataagggatgtgatttgtcttttgggaactcatctttgattcatcattgtctcttggtatcttggaatttccatgtcattacagtctacagaatgaaagagtaacctgtcccagaggagaggcaggtgaaagactccacagcatgctcattctcattctgtcttctcagtgacaccgaggtttactgagtgcccactatgtgccaagcactgtgctcagggctttctttgtatgcatgatctcagtgaatctcaccaagcctcatctggaaaacggggacaaattaacaacaggatggcaaattgaaaaacacgtaaccatgttctacagatggaaaggggtgcttggttattatgaaggccccctcgcaagcgtgtgggacatgggtgtgttctctgggttgtactgatcagatcaaggacctcccccacccttctcacactctgcccacttccgccctttgcttatcagacccttagccagtgactcattccagaaccagaaccttggtgaaatctcaaccgacaccagagatcggtgtcttcagtcctagactgatggagaaaatccagaatatatactagaagctccaaatgctctgggtttcagctcctctgtgctgtggacactgactttggctcagaactccgatttagtacaaaaggctcatttttatttcaggggcactcttcctaaagcaaacctaataaatgaaatatggaattcacagatacacacacacattaaaaaattaacctagtgtatctgtgaggagtaggcagaaattcactgtataaaagaatgcttcatttcatagagaatttgtgttaagattccattagatagtacatttctcaaagatttttgaggttgtatttgctttaccaaaacttggtttatgtaagtggaaaaagcatgttgcaaaataacttggtgtctatgattcagtttatgtaaaataataaatgtatgtaggaatacgtgtgttgaaagatgtacatcaatttgctaacaatggttatctctgacgtggtgggatttgagatgtgtttttctttttggttgtatttttctctattgtttgacttaacacagaacatgcttggttacaacaataaagttattgaagacaaaaaaaaaaaaaaaaaaaaa

SEQ ID NO: 7 - DNA, 863 bp, Homo sapiens
caaggttcagagtcacccatctcagcaagcccagaagtatctgcaatatctacgatggcctcgccctttgctttactgatggtcctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataacatctggtccaacatgaaaacaattcttattgactcatacaccaggtcacgctttcatgaattctgtcatttcaaagactctcacccctgctataactatgaccatgctgataaactgatttatctatttaaatatttatttaactattcataagatttaaattatttttgttcatataacgtcatgtgcacctttacactgtggttagtgtaataaaacatgttccttatatttactc

SEQ ID NO: 8 - protein, 189 aa, Homo sapiens
MASPFALLMVLVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 9 - DNA, 1143 bp, Homo sapiens
gagaacctggagcctaaggtttaggctcacccatttcaaccagtctagcagcatctgcaacatctacaatggccttgacctttgctttactggtggccctcctggtgctcagctgcaagtcaagctgctctgtgggctgtgatctgcctcaaacccacagcctgggtagcaggaggaccttgatgctcctggcacagatgaggagaatctctcttttctcctgcttgaaggacagacatgactttggatttccccaggaggagtttggcaaccagttccaaaaggctgaaaccatccctgtcctccatgagatgatccagcagatcttcaatctcttcagcacaaaggactcatctgctgcttgggatgagaccctcctagacaaattctacactgaactctaccagcagctgaatgacctggaagcctgtgtgatacagggggtgggggtgacagagactcccctgatgaaggaggactccattctggctgtgaggaaatacttccaaagaatcactctctatctgaaagagaagaaatacagcccttgtgcctgggaggttgtcagagcagaaatcatgagatctttttctttgtcaacaaacttgcaagaaagtttaagaagtaaggaatgaaaactggttcaacatggaaatgattttcattgattcgtatgccagctcacctttttatgatctgccatttcaaagactcatgtttctgctatgaccatgacacgatttaaatcttttcaaatgtttttaggagtattaatcaacattgtattcagctcttaaggcactagtcccttacagaggaccatgctgactgatccattatctatttaaatatttttaaaatattatttatttaactatttataaaacaacttatttttgttcatattatgtcatgtgcacctttgcacagtggttaatgtaataaaatatgttctttgtatttggtaaatttattttgtgttgttcattgaacttttgctatggaaacttttgtacttgtttattctttaaaatgaaattccaagcctaattgtgcaacctgattacagaataactggtacacttcatttatccatcaatattatattcaagatataagtaaaaataaactttctgtaaacca

SEQ ID NO: 10 - protein, 188 aa, Homo sapiens
MALTFALLVALLVLSCKSSCSVGCDLPQTHSLGSRRTLMLLAQMRRISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 11 - DNA, 982 bp, Homo sapiens
agaaaacctagaggccgaagttcaaggttatccatctcaagtagcctagcaatatttgcaacatcccaatggccctgtccttttctttactgatggccgtgctggtgctcagctacaaatccatctgttctctgggctgtgatctgcctcagacccacagcctgggtaataggagggccttgatactcctggcacaaatgggaagaatctctcatttctcctgcctgaaggacagacatgatttcggattccccgaggaggagtttgatggccaccagttccagaaggctcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggactccatcctggctgtgaggaaatacttccaaagaatcactctttatctaacagagaagaaatacagcccttgtgcctgggaggttgtcagagcagaaatcatgagatccctctcgttttcaacaaacttgcaaaaaagattaaggaggaaggattgaaacctggttcaacatggaaatgatcctgattgactaatacattatctcacactttcatgagttcttccatttcaaagactcacttctataaccaccacgagttgaatcaaaattttcaaatgttttcagcagtgtgaagaagcttggtgtatacctgtgcaggcactagtcctttacagatgacaatgctgatgtctctgttcatctatttatttaaatatttatttatttttaaaatttaaattattttttatgtgatatcatgagtacctttacattgtggtgaatgtaacaatatatgttcttcatatttagccaatatattaatttcctttttcattaaa

SEQ ID NO: 12 - protein, 189 aa, Homo sapiens
MALSFSLLMAVLVLSYKSICSLGCDLPQTHSLGNRRALILLAQMGRISHFSCLKDRHDFGFPEEEFDGHQFQKAQAISVLHEMIQQTFNLFSTEDSSAAWEQSLLEKFSTELYQQLNDLEACVIQEVGVEETPLMNEDSILAVRKYFQRITLYLTEKKYSPCAWEVVRAEIMRSLSFSTNLQKRLRRKD

SEQ ID NO: 13 - DNA, 700 bp, Homo sapiens
gcccaaggttcagggtcactcaatctcaacagcccagaagcatctgcaacctccccaatggccttgccctttgttttactgatggccctggtggtgctcaactgcaagtcaatctgttctctgggctgtgatctgcctcagacccacagcctgagtaacaggaggactttgatgataatggcacaaatgggaagaatctctcctttctcctgcctgaaggacagacatgactttggatttcctcaggaggagtttgatggcaaccagttccagaaggctcaagccatctctgtcctccatgagatgatccagcagaccttcaatctcttcagcacaaaggactcatctgctacttgggatgagacacttctagacaaattctacactgaactttaccagcagctgaatgacctggaagcctgtatgatgcaggaggttggagtggaagacactcctctgatgaatgtggactctatcctgactgtgagaaaatactttcaaagaatcaccctctatctgacagagaagaaatacagcccttgtgcatgggaggttgtcagagcagaaatcatgagatccttctctttatcagcaaacttgcaagaaagattaaggaggaaggaatgaaaactggttcaacatcgaaatgattctcattgactagtacaccatttcacacttcttgagttctgccgtttca

SEQ ID NO: 14 - protein, 189 aa, Homo sapiens
MALPFVLLMALVVLNCKSICSLGCDLPQTHSLSNRRTLMIMAQMGRISPFSCLKDRHDFGFPQEEFDGNQFQKAQAISVLHEMIQQTFNLFSTKDSSATWDETLLDKFYTELYQQLNDLEACMMQEVGVEDTPLMNVDSILTVRKYFQRITLYLTEKKYSPCAWEVVRAEIMRSFSLSANLQERLRRKE

SEQ ID NO: 15 - DNA, 570 bp, Homo sapiens
atggctttgccttttgctttactgatggccctggtggtgctcagctgcaagtcaagctgctctctggactgtgatctgcctcagacccacagcctgggtcacaggaggaccatgatgctcctggcacaaatgaggagaatctctcttttctcctgtctgaaggacagacatgacttcagatttccccaggaggagtttgatggcaaccagttccagaaggctgaagccatctctgtcctccatgaggtgattcagcagaccttcaacctcttcagcacaaaggactcatctgttgcttgggatgagaggcttctagacaaactctatactgaactttaccagcagctgaatgacctggaagcctgtgtgatgcaggaggtgtgggtgggagggactcccctgatgaatgaggactccatcctggctgtgagaaaatacttccaaagaatcactctctacctgacagagaaaaagtacagcccttgtgcctgggaggttgtcagagcagaaatcatgagatccttctcttcatcaagaaacttgcaagaaaggttaaggaggaaggaataa

SEQ ID NO: 16 - protein, 189 aa, Homo sapiens
MALPFALLMALVVLSCKSSCSLDCDLPQTHSLGHRRTMMLLAQMRRISLFSCLKDRHDFRFPQEEFDGNQFQKAEAISVLHEVIQQTFNLFSTKDSSVAWDERLLDKLYTELYQQLNDLEACVMQEVWVGGTPLMNEDSILAVRKYFQRITLYLTEKKYSPCAWEVVRAEIMRSFSSSRNLQERLRRKE

SEQ ID NO: 17 - DNA, 736 bp, Homo sapiens
tacccacctcaggtagcctagtgatatttgcaaaatcccaatggcccggtccttttctttactgatggtcgtgctggtactcagctacaaatccatctgctctctgggctgtgatctgcctcagacccacagcctgcgtaataggagggccttgatactcctggcacaaatgggaagaatctctcctttctcctgcttgaaggacagacatgaattcagattcccagaggaggagtttgatggccaccagttccagaagactcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggacttcatcctggctgtgaggaaatacttccaaagaatcactctttatctaatggagaagaaatacagcccttgtgcctgggaggttgtcagagcagaaatcatgagatccttctctttttcaacaaacttgaaaaaaggattaaggaggaaggattgaaaactggttcatcatggaaatgattctcattgactaatgcatcatctcacactttcatgagttcttccatttcaaagactcacttctataaccaccacaagttaatcaaaatttccaaatgttttc

SEQ ID NO: 18 - protein, 189 aa, Homo sapiens
MARSFSLLMVVLVLSYKSICSLGCDLPQTHSLRNRRALILLAQMGRISPFSCLKDRHEFRFPEEEFDGHQFQKTQAISVLHEMIQQTFNLFSTEDSSAAWEQSLLEKFSTELYQQLNDLEACVIQEVGVEETPLMNEDFILAVRKYFQRITLYLMEKKYSPCAWEVVRAEIMRSFSFSTNLKKGLRRKD

SEQ ID NO: 19 - DNA, 1039 bp, Homo sapiens
accagctcagcagcatccacaacatctacaatggccttgactttttatttactggtggccctagtggtgctcagctacaagtcattcagctctctgggctgtgatctgcctcagactcacagcctgggtaacaggagggccttgatactcctggcacaaatgcgaagaatctctcctttctcctgcctgaaggacagacatgactttgaattcccccaggaggagtttgatgataaacagttccagaaggctcaagccatctctgtcctccatgagatgatccagcagaccttcaacctcttcagcacaaaggactcatctgctgctttggatgagacccttctagatgaattctacatcgaacttgaccagcagctgaatgacctggagtcctgtgtgatgcaggaagtgggggtgatagagtctcccctgatgtacgaggactccatcctggctgtgaggaaatacttccaaagaatcactctatatctgacagagaagaaatacagctcttgtgcctgggaggttgtcagagcagaaatcatgagatccttctctttatcaatcaacttgcaaaaaagattgaagagtaaggaatgagacctggtacaacacggaaatgattcttatagactaatacagcagctcacacttcgacaagttgtgctctttcaaagacccttgtttctgccaaaaccatgctatgaattgaatcaaatgtgtcaagtgttttcaggagtgttaagcaacatcctgttcagctgtatgggcactagtcccttacagatgaccatgctgatggatctattcatctatttatttaaatctttatttagttaactatctatagggcttaaattagttttgttcatattatattatgtgaacttttacattgtgaattgtgtaacaaaaacatgttctttatatttattattttgccttgtttattaaatttttactatagaaaaattctttatttattctttaaaattgaactccaaccctgattgtgcaaactgattaaagaatggatggt

SEQ ID NO: 20 - protein, 189 aa, Homo sapiens
MALTFYLLVALVVLSYKSFSSLGCDLPQTHSLGNRRALILLAQMRRISPFSCLKDRHDFEFPQEEFDDKQFQKAQAISVLHEMIQQTFNLFSTKDSSAALDETLLDEFYIELDQQLNDLESCVMQEVGVIESPLMYEDSILAVRKYFQRITLYLTEKKYSSCAWEVVRAEIMRSFSLSINLQKRLKSKE

SEQ ID NO: 21 - DNA, 963 bp, Homo sapiens
caaggttatccatctcaagtagcctagcaatatttgcaacatcccaatggccctgtccttttctttacttatggccgtgctggtgctcagctacaaatccatctgttctctgggctgtgatctgcctcagacccacagcctcggtaataggagggccttgatactcctgggacaaatgggaagaatctctcctttctcctgcctgaaggacagacatgatttccgaatcccccaggaggagtttgatggcaaccagttccagaaggctcaagccatctctgtcctccatgagatgatccagcagaccttcaatctcttcagcacagaggactcatctgctgcttgggaacagagcctcctagaaaaattttccactgaactttaccagcaactgaatgacctggaagcatgtgtgatacaggaggttggggtggaagagactcccctgatgaatgaggactccatcctggctgtgaggaaatacttccaaagaatcactctttatctaatagagaggaaatacagcccttgtgcctgggaggttgtcagagcagaaatcatgagatccctctcgttttcaacaaacttgcaaaaaagattaaggaggaaggattgaaaactggttcaacatggcaatgatcctgattgactaatacattatctcacactttcatgagttcttccatttcaaagactcacttctataaccacgacgcgttgaatcaaaattttcaaatgttttcagcagtgtaaagaagtgtcgtgtatacctgtgcaggcactagtcctttacagatgaccattctgatgtctctgttcatcttttgtttaaatatttatttaattatttttaaaatttatgtaatatcatgagtcgctttacattgtggttaatgtaacaatatatgttcttcatatttagccaatatattaatttcctttttcattaaatttttactatac

SEQ ID NO: 22 - protein, 189 aa, Homo sapiens
MALSFSLLMAVLVLSYKSICSLGCDLPQTHSLGNRRALILLGQMGRISPFSCLKDRHDFRIPQEEFDGNQFQKAQAISVLHEMIQQTFNLFSTEDSSAAWEQSLLEKFSTELYQQLNDLEACVIQEVGVEETPLMNEDSILAVRKYFQRITLYLIERKYSPCAWEVVRAEIMRSLSFSTNLQKRLRRKD

SEQ ID NO: 23 - DNA, 705 bp, Homo sapiens
agagaacctagagcccaaggttcagagtcacccatctcagcaagcccagaagcatctgcaatatctatgatggcctcgccctttgctttactgatggccctggtggtgctcagctgcaagtcaagctgctctctgggctgtgatctccctgagacccacagcctggataacaggaggaccttgatgctcctggcacaaatgagcagaatctctccttcctcctgtctgatggacagacatgactttggatttccccaggaggagtttgatggcaaccagttccagaaggctccagccatctctgtcctccatgagctgatccagcagatcttcaacctctttaccacaaaagattcatctgctgcttgggatgaggacctcctagacaaattctgcaccgaactctaccagcagctgaatgacttggaagcctgtgtgatgcaggaggagagggtgggagaaactcccctgatgaatgcggactccatcttggctgtgaagaaatacttccgaagaatcactctctatctgacagagaagaaatacagcccttgtgcctgggaggttgtcagagcagaaatcatgagatccctctctttatcaacaaacttgcaagaaagattaaggaggaaggaataacacctggtccaacatgaaacaattcttattgactcatataccaggtcacgctttcatgaattctgc

SEQ ID NO: 24 - protein, 190 aa, Homo sapiens
MMASPFALLMALVVLSCKSSCSLGCDLPETHSLDNRRTLMLLAQMSRISPSSCLMDRHDFGFPQEEFDGNQFQKAPAISVLHELIQQIFNLFTTKDSSAAWDEDLLDKFCTELYQQLNDLEACVMQEERVGETPLMNADSILAVKKYFRRITLYLTEKKYSPCAWEVVRAEIMRSLSLSTNLQERLRRKE

SEQ ID NO: 25 - DNA, 778 bp, Homo sapiens
gttacccctcatcaaccagcccagcagcatcttcgggattcccaatggcattgccctttgctttaatgatggccctggtggtgctcagctgcaagtcaagctgctctctgggctgtaatctgtctcaaacccacagcctgaataacaggaggactttgatgctcatggcacaaatgaggagaatctctcctttctcctgcctgaaggacagacatgactttgaatttccccaggaggaatttgatggcaaccagttccagaaagctcaagccatctctgtcctccatgagatgatgcagcagaccttcaatctcttcagcacaaagaactcatctgctgcttgggatgagaccctcctagaaaaattctacattgaacttttccagcaaatgaatgacctggaagcctgtgtgatacaggaggttggggtggaagagactcccctgatgaatgaggactccatcctggctgtgaagaaatacttccaaagaatcactctttatctgatggagaagaaatacagcccttgtgcctgggaggttgtcagagcagaaatcatgagatccctctctttttcaacaaacttgcaaaaaagattaaggaggaaggattgaaaactggttcatcatggaaatgattctcattgactaatacatcatctcacactttcatgagttcttccatttcaaagactcacttctcctataaccaccacaagttgaatcaaaattttcaaatgttttcaggagtgtaaagaagcatcatgtatacctgtgca

SEQ ID NO: 26 - protein, 189 aa, Homo sapiens
MALPFALMMALVVLSCKSSCSLGCNLSQTHSLNNRRTLMLMAQMRRISPFSCLKDRHDFEFPQEEFDGNQFQKAQAISVLHEMMQQTFNLFSTKNSSAAWDETLLEKFYIELFQQMNDLEACVIQEVGVEETPLMNEDSILAVKKYFQRITLYLMEKKYSPCAWEVVRAEIMRSLSFSTNLQKRLRRKD

SEQ ID NO: 27 - DNA, 939 bp, Homo sapiens
atcccaatggccctgtccttttctttactgatggccgtgctggtgctcagctacaaatccatctgttctctgggctgtgatctgcctcagactcacagcctgggtaataggagggccttgatactcctggcacaaatgggaagaatctctcatttctcctgcctgaaggacagatatgatttcggattcccccaggaggtgtttgatggcaaccagttccagaaggctcaagccatctctgccttccatgagatgatccagcagaccttcaatctcttcagcacaaaggattcatctgctgcttgggatgagaccctcctagacaaattctacattgaacttttccagcaactgaatgacctagaagcctgtgtgacacaggaggttggggtggaagagattgccctgatgaatgaggactccatcctggctgtgaggaaatactttcaaagaatcactctttatctgatggggaagaaatacagcccttgtgcctgggaggttgtcagagcagaaatcatgagatccttctctttttcaacaaacttgcaaaaaggattaagaaggaaggattgaaaactcattcaacatggaaatgatcctcattgattaatacatcatctcacactttcatgagttcttccatttcaaagactcacttctataaccaccacaagttgaatcaaaatttcaaaatgttttcaggagtgtaaagaagcatcgtgtttacctgtgcaggcactagtcctttacagatgaccatgctgatgtctctattcatctatttatttaaatatttatttatttaactatttttaaggtttaaatcatgttttatgtaatatcatgtgtacctttacattttgcttaatgtaacaatatatgttcttcatatttagttaatatattaacttccttttcattaaatttttactatac

SEQ ID NO: 28 - protein, 189 aa, Homo sapiens
MALSFSLLMAVLVLSYKSICSLGCDLPQTHSLGNRRALILLAQMGRISHFSCLKDRYDFGFPQEVFDGNQFQKAQAISAFHEMIQQTFNLFSTKDSSAAWDETLLDKFYIELFQQLNDLEACVTQEVGVEEIALMNEDSILAVRKYFQRITLYLMGKKYSPCAWEVVRAEIMRSFSFSTNLQKGLRRKD

SEQ ID NO: 29 - DNA, 980 bp, Homo sapiens
gttcaaggttacccatctcaagtagcctagcaacatttgcaacatcccaatggccctgtccttttctttactgatggccgtgctggtgctcagctacaaatccatctgttctctaggctgtgatctgcctcagacccacagcctgggtaataggagggccttgatactcctggcacaaatgggaagaatctctcctttctcctgcctgaaggacagacatgactttggacttccccaggaggagtttgatggcaaccagttccagaagactcaagccatctctgtcctccatgagatgatccagcagaccttcaatctcttcagcacagaggactcatctgctgcttgggaacagagcctcctagaaaaattttccactgaactttaccagcaactgaataacctggaagcatgtgtgatacaggaggttgggatggaagagactcccctgatgaatgaggactccatcctggctgtgaggaaatacttccaaagaatcactctttatctaacagagaagaaatacagcccttgtgcctgggaggttgtcagagcagaaatcatgagatctctctctttttcaacaaacttgcaaaaaatattaaggaggaaggattgaaaactggttcaacatggcaatgatcctgattgactaatacattatctcacactttcatgagttcctccatttcaaagactcacttctataaccaccacgagttgaatcaaaattttcaaatgttttcagcagtgtaaagaagcgtcgtgtatacctgtgcaggcactagtactttacagatgaccatgctgatgtctctgttcatctatttatttaaatatttatttaattatttttaagatttaaattatttttttatgtaatatcatgtgtacctttacattgtggtgaatgtaacaatatatgttcttcatatttagccaatatattaatttcctttttcattaaatttttactatac

SEQ ID NO: 30 - protein, 189 aa, Homo sapiens
MALSFSLLMAVLVLSYKSICSLGCDLPQTHSLGNRRALILLAQMGRISPFSCLKDRHDFGLPQEEFDGNQFQKTQAISVLHEMIQQTFNLFSTEDSSAAWEQSLLEKFSTELYQQLNNLEACVIQEVGMEETPLMNEDSILAVRKYFQRITLYLTEKKYSPCAWEVVRAEIMRSLSFSTNLQKILRRKD

SEQ ID NO: 31 - DNA, 1024 bp, Homo sapiens
ttcaaggttacccatctcaagtagcctagcaatattggcaacatcccaatggccctgtccttttctttactgatggccgtgctggtgctcagctacaaatccatctgttctctgggctgtgatctgcctcagacccacagcctgggtaataggagggccttgatactcctggcacaaatgggaagaatctctcctttctcctgcctgaaggacagacatgactttggattcccccaggaggagtttgatggcaaccagttccagaaggctcaagccatctctgtcctccatgagatgatccagcagaccttcaatctcttcagcacaaaggactcatctgctacttgggaacagagcctcctagaaaaattttccactgaacttaaccagcagctgaatgacctggaagcctgcgtgatacaggaggttggggtggaagagactcccctgatgaatgtggactccatcctggctgtgaagaaatacttccaaagaatcactctttatctgacagagaagaaatacagcccttgtgcctgggaggttgtcagagcagaaatcatgagatccttctctttatcaaaaatttttcaagaaagattaaggaggaaggaatgaaacctgtttcaacatggaaatgatctgtattgactaatacaccagtccacacttctatgacttctgccatttcaaagactcatttctcctataaccaccgcatgagttgaatcaaaattttcagatcttttcaggagtgtaaggaaacatcatgtttacctgtgcaggcactagtcctttacagatgaccatgctgatagatctaattatctatctattgaaatatttatttatttattagatttaaattatttttgtccatgtaatattatgtgtacttttacattgtgttatatcaaaatatgttatttatatttagtcaatatattattttctttttattaatttttactattaaaacttcttatattatttgtttattctttaataaagaaataccaagccc

SEQ ID NO: 32 - protein, 189 aa, Homo sapiens
MALSFSLLMAVLVLSYKSICSLGCDLPQTHSLGNRRALILLAQMGRISPFSCLKDRHDFGFPQEEFDGNQFQKAQAISVLHEMIQQTFNLFSTKDSSATWEQSLLEKFSTELNQQLNDLEACVIQEVGVEETPLMNVDSILAVKKYFQRITLYLTEKKYSPCAWEVVRAEIMRSFSLSKIFQERLRRKE

SEQ ID NO: 33 - DNA, 840 bp, Homo sapiens
acattctaactgcaacctttcgaagcctttgctctggcacaacaggtagtaggcgacactgttcgtgttgtcaacatgaccaacaagtgtctcctccaaattgctctcctgttgtgcttctccactacagctctttccatgagctacaacttgcttggattcctacaaagaagcagcaattttcagtgtcagaagctcctgtggcaattgaatgggaggcttgaatactgcctcaaggacaggatgaactttgacatccctgaggagattaagcagctgcagcagttccagaaggaggacgccgcattgaccatctatgagatgctccagaacatctttgctattttcagacaagattcatctagcactggctggaatgagactattgttgagaacctcctggctaatgtctatcatcagataaaccatctgaagacagtcctggaagaaaaactggagaaagaagatttcaccaggggaaaactcatgagcagtctgcacctgaaaagatattatgggaggattctgcattacctgaaggccaaggagtacagtcactgtgcctggaccatagtcagagtggaaatcctaaggaacttttacttcattaacagacttacaggttacctccgaaactgaagatctcctagcctgtgcctctgggactggacaattgcttcaagcattcttcaaccagcagatgctgtttaagtgactgatggctaatgtactgcatatgaaaggacactagaagattttgaaatttttattaaattatgagttatttttatttatttaaattttattttggaaaataaattatttttggtgcaaaagtca

SEQ ID NO: 34 - protein, 187 aa, Homo sapiens
MTNKCLLQIALLLCFSTTALSMSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 35 - DNA, 1514 bp, Homo sapiens
gatctggtaaacctgaagcaaatatagaaacctatagggcctgacttcctacataaagtaaggagggtaaaaatggaggctagaataagggttaaaattttgcttctagaacagagaaaatgatttttttcatatatatatgaatatatattatatatacacatatatacatatattcactatagtgtgtatacataaatatataatatatatattgttagtgtagtgtgtgtctgattatttacatgcatatagtatatacacttatgactttagtacccagacgtttttcatttgattaagcattcatttgtattgacacagctgaagtttactggagtttagctgaagtctaatgcaaaattaatagattgttgtcatcctcttaaggtcatagggagaacacacaaatgaaaacagtaaaagaaactgaaagtacagagaaatgttcagaaaatgaaaaccatgtgtttcctattaaaagccatgcatacaagcaatgtcttcagaaaacctagggtccaaggttaagccatatcccagctcagtaaagccaggagcatcctcatttcccaatggccctcctgttccctctactggcagccctagtgatgaccagctatagccctgttggatctctgggctgtgatctgcctcagaaccatggcctacttagcaggaacaccttggtgcttctgcaccaaatgaggagaatctcccctttcttgtgtctcaaggacagaagagacttcaggttcccccaggagatggtaaaagggagccagttgcagaaggcccatgtcatgtctgtcctccatgagatgctgcagcagatcttcagcctcttccacacagagcgctcctctgctgcctggaacatgaccctcctagaccaactccacactggacttcatcagcaactgcaacacctggagacctgcttgctgcaggtagtgggagaaggagaatctgctggggcaattagcagccctgcactgaccttgaggaggtacttccagggaatccgtgtctacctgaaagagaagaaatacagcgactgtgcctgggaagttgtcagaatggaaatcatgaaatccttgttcttatcaacaaacatgcaagaaagactgagaagtaaagatagagacctgggctcatcttgaaatgattctcattgattaatttgccatataacacttgcacatgtgactctggtcaattcaaaagactcttatttcggctttaatcacagaattgactgaattagttctgcaaatactttgtcggtatattaagccagtatatgttaaaaagacttaggttcaggggcatcagtccctaagatgttatttatttttactcatttatttattcttacattttatcatatttatactatttatattcttatataacaaatgtttgcctttacattgtattaagataacaaaacatgttcagctttccatttggttaaatattgtattttgttatttattaaattattttcaaac

SEQ ID NO: 36 - protein, 195 aa, Homo sapiens
MALLFPLLAALVMTSYSPVGSLGCDLPQNHGLLSRNTLVLLHQMRRISPFLCLKDRRDFRFPQEMVKGSQLQKAHVMSVLHEMLQQIFSLFHTERSSAAWNMTLLDQLHTGLHQQLQHLETCLLQVVGEGESAGAISSPALTLRRYFQGIRVYLKEKKYSDCAWEVVRMEIMKSLFLSTNMQERLRSKDRDLGSS

SEQ ID NO: 37 - DNA, 1240 bp, Homo sapiens
cacattgttctgatcatctgaagatcagctattagaagagaaagatcagttaagtcctttggacctgatcagcttgatacaagaactactgatttcaacttctttggcttaattctctcggaaacgatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaatggttgtcctgcctgcaatatttgaattttaaatctaaatctatttattaatatttaacattatttatatggggaatatatttttagactcatcaatcaaataagtatttataatagcaacttttgtgtaatgaaaatgaatatctattaatatatgtattatttataattcctatatcctgtgactgtctcacttaatcctttgttttctgactaattaggcaaggctatgtgattacaaggctttatctcaggggccaactaggcagccaacctaagcaagatcccatgggttgtgtgtttatttcacttgatgatacaatgaacacttataagtgaagtgatactatccagttactgccggtttgaaaatatgcctgcaatctgagccagtgctttaatggcatgtcagacagaacttgaatgtgtcaggtgaccctgatgaaaacatagcatctcaggagatttcatgcctggtgcttccaaatattgttgacaactgtgactgtacccaaatggaaagtaactcatttgttaaaattatcaatatctaatatatatgaataaagtgtaagttcacaacaaaaaaaaaaaaaaaaaaaaaaaaaaaaaa

SEQ ID NO: 38 - protein, 166 aa, Homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 39 - DNA, 5889 bp, Homo sapiens
gctgccagctgagtttttttgctgctttgagtctcagttttctttctttcctagagtctctgaagccacagatctcttaagaactttctgtctccaaaccgtggctgctcgataaatcagacagaacagttaatcctcaatttaagcctgatctaacccctagaaacagatatagaacaatggaagtgacaacaagattgacatggaatgatgaaaatcatctgcgcaagctgcttggaaatgtttctttgagtcttctctataagtctagtgttcatggaggtagcattgaagatatggttgaaagatgcagccgtcagggatgtactataacaatggcttacattgattacaatatgattgtagcctttatgcttggaaattatattaatttacatgaaagttctacagagccaaatgattccctatggttttcacttcaaaagaaaaatgacaccactgaaatagaaactttactcttaaatacagcaccaaaaattattgatgagcaactggtgtgtcgtttatcgaaaacggatattttcattatatgtcgagataataaaatttatctagataaaatgataacaagaaacttgaaactaaggttttatggccaccgtcagtatttggaatgtgaagtttttcgagttgaaggaattaaggataacctagacgacataaagaggataattaaagccagagagcacagaaataggcttctagcagacatcagagactataggccctatgcagacttggtttcagaaattcgtattcttttggtgggtccagttgggtctggaaagtccagttttttcaattcagtcaagtctatttttcatggccatgtgactggccaagccgtagtggggtctgatatcaccagcataaccgagcggtataggatatattctgttaaagatggaaaaaatggaaaatctctgccatttatgttgtgtgacactatggggctagatggggcagaaggagcaggactgtgcatggatgacattccccacatcttaaaaggttgtatgccagacagatatcagtttaattcccgtaaaccaattacacctgagcattctacttttatcacctctccatctctgaaggacaggattcactgtgtggcttatgtcttagacatcaactctattgacaatctctactctaaaatgttggcaaaagtgaagcaagttcacaaagaagtattaaactgtggtatagcatatgtggccttgcttactaaagtggatgattgcagtgaggttcttcaagacaactttttaaacatgagtagatctatgacttctcaaagccgggtcatgaatgtccataaaatgctaggcattcctatttccaatattttgatggttggaaactatgcttcagatttggaactggaccccatgaaggatattctcatcctctctgcactgaggcagatgctgcgggctgcagatgattttttagaagatttgcctcttgaggaaactggtgcaattgagagagcgttacagccctgcatttgagataagttgccttgattctgacatttggcccagcctgtactggtgtgccgcaatgagagtcaatctctattgacagcctgcttcagattttgcttttgttcgttttgccttctgtccttggaacagtcatatctcaagttcaaaggccaaaacctgagaagcggtgggctaagataggtcctactgcaaaccacccctccatatttccgtaccatttacaattcagtttctgtgacatctttttaaaccactggaggaaaaatgagatattctctaatttattcttctataacactctatatagagctatgtgagtactaatcacattgaataatagttataaaattattgtatagacatctgcttcttaaacagattgtgagttctttgagaaacagcgtggattttacttatctgtgtattcacagagcttagcacagtgcctggtaatgagcaagcatacttgccattacttttccttcccactctctccaacatcacattcactttaaatttttctgtatatagaaaggaaaactagcctgggcaacatgatgaaaccccatctccactgcaaaaaaaaaaaaaaaaaataagaaagaacaaaacaaaccccacaaaaattagctgggtatgatggcacgtgcctgtagtcccagttactcaggatgattgattgagccttggaggtggaggctacagtgagctgagattgtgccactgtactctagccagggagaaagagtgagatcctggctcaaaaaaaccaaataaaacaaaacaaacaaacgaaaaacagaaaggaagactgaaagagaatgaaaagctggggagaggaaataaaaataaagaaggaagagtgtttcatttatatctgaatgaaaatatgaatgactctaagtaattgaattaattaaaatgagccaacttttttttaacaatttacattttatttctatgggaaaaaataaatattcctcttctaacaaacccatgcttgattttcattaattgaattccaaatcatcctagccatgtgtccttccatttaggttactggggcaaatcagtaagaaagttcttatatttatgctccaaataattctgaagtcctcttactagctgtgaaagctagtactattaagaaagaaaacaaaattcccaaaagatagctttcacttttttttttccttaaagacttcctaattctcttctccaaattcttagtcttcttcaaaataatatgctttggttcaatagttatccacattctgacagtctaatttagttttaatcagaattatactcatcttttgggtagtcatagatattaagaaagcaagagtttcttatgtccagttatggaatatttcctaaagcaaggctgcaggtgaagttgtgctcaagtgaatgttcaggagacacaattcagtggaagaaattaagtctttaaaaaagacctaggaataggagaaccatggaaattgaggaggtaggcctacaagtagatattgggaacaaaattagagaggcaaccagaaaaagttattttaggctcaccagagttgttcttattgcacagtaacacaccaatataccaaaacagcaggtattgcagtagagaaagagtttaataattgaatggcagaaaaatgaggaaggttgaggaaacctcaaatctacctccctgctgagtctaagtttaggatttttaagagaaaggcaggtaaggtgctgaaggtctggagctgctgatttgttggggtatagggaatgaaatgaaacatacagagatgaaaactggaagtttttttttgtttgttttgttttttttttgttgttgtttttttttttttttgtttttttgctgagtcaattccttggagggggtcttcagactgactggtgtcagcagacccatgggattccaagatctggaaaactttttagatagaaacttgatgtttcttaacgttacatatattatcttatagaaataactaagggaagttagtgccttgtgaccacatctatgtgacttttaggcagtaagaaactataaggaaaggagctaacagtcatgctgtaagtagctacagggaattggcttaaagggcaagttggttagtacttagctgtgtttttattcaaagtctacattttatgtagtggttaatgtttgctgttcattaggatggtttcacagttaccatacaaatgtagaagcaacaggtccaaaaagtagggcatgattttctccatgtaatccagggagaaaacaagccatgaccattgttggttgggagactgaaggtgattgaaggttcaccatcatcctcaccaacttttgggccataattcacccaaccctttggtggagcctgaaaaaaatctgggcagaatgtaggacttctttattttgtttaaaggggtaacacagagtgcccttatgaaggagttggagatcctgcaaggaagagaaggagtgaaggagagatcaagagagagaaacaatgaggaacatttcatttgacccaacatcctttaggagcataaatgttgacactaagttatcccttttgtgctaaaatggacagtattggcaaaatgataccacaacttcttattctctggctctatattgctttggaaacacttaaacatcaaatggagttaaatacatatttgaaatttaggttaggaaatattggtgaggaggcctcaaaaagggggaaacatcttttgtctgggaggatattttccattttgtggatttccctgatctttttctaccaccctgaggggtggtgggaattatcattttgctacattttagaggtcatccaggatttttgaaactttacattctttacggttaagcaagatgtacagctcagtcaaagacactaaattcttcttagaaaaatagtgctaaggagtatagcagatgacctatatgtgtgttggctgggagaatatcatcttaaagtgagagtgatgttgtggagacagttgaaatgtcaatgctagagcctctgtggtgtgaatgggcacgttaggttgttgcattagaaagtgactgtttctgacagaaatttgtagctttgtgcaaactcacccaccatctacctcaataaaatatagagaaaagaaaaatagagcagtttgagttctatgaggtatgcaggcccagagagacataagtatgttcctttagtcttgcttcctgtgtgccacactgcccctccacaaccatagctgggggcaattgtttaaagtcattttgttcccgactagctgccttgcacattatcttcattttcctggaatttgatacagagagcaatttatagccaattgatagcttatgctgtttcaatgtaaattcgtggtaaataacttaggaactgcctcttctttttctttgaaaacctacttataactgttgctaataagaatgtgtattgttcaggacaacttgtctccatacagttgggttgtaaccctcatgcttggcccaaataaactctctacttatatcagtttttcctacacttcttccttttaggtcaacaataccaagaggggttactgtgctgggtaatgtgtaaacttgtgtcttgtttagaaagataaatttaaagactatcacattgctttttcataaaacaagacaggtctacaattaatttattttgacgcaaattgataggggggccaagtaagccccatatgcttaatgatcagctgatgaataatcatctcctagcaacataactcaatctaatgctaaggtacccacaagatggcaaggctgatcaaagtcgtcatggaatcctgcaaccaaaagccatgggaatttggaagccctcaaatcccattcctaatctgatgagtctatggaccaatttgtggaggacagtagattaaatagatctgatttttgccatcaatgtaaggaggataaaaacttgcataccaattgtacacccttgcaaaatctttctctgatgttggagaaaatgggccagtgagatcatggatatagaagtacagtcaatgttcagctgtaccctcccacaatcccacttccttcctcaacacaattcaaacaaatagactcagactgtttcaggctccaggacaggaagtgcagtgtaggcaaaattgcaaaaattgagggcacaggggtggaggtgggggggttgaataacaagctgtgctaaataattacgtgtaaatatattttttcatttttaaaaattgatttcttttgcacattccatgacaatatatgtcacatttttaaaataaatgcaaagaagcatacatccaaaaaaaaaaaaaaaaa

SEQ ID NO: 40 - protein, 452 aa, Homo sapiens
MEVTTRLTWNDENHLRKLLGNVSLSLLYKSSVHGGSIEDMVERCSRQGCTITMAYIDYNMIVAFMLGNYINLHESSTEPNDSLWFSLQKKNDTTEIETLLLNTAPKIIDEQLVCRLSKTDIFIICRDNKIYLDKMITRNLKLRFYGHRQYLECEVFRVEGIKDNLDDIKRIIKAREHRNRLLADIRDYRPYADLVSEIRILLVGPVGSGKSSFFNSVKSIFHGHVTGQAVVGSDITSITERYRIYSVKDGKNGKSLPFMLCDTMGLDGAEGAGLCMDDIPHILKGCMPDRYQFNSRKPITPEHSTFITSPSLKDRIHCVAYVLDINSIDNLYSKMLAKVKQVHKEVLNCGIAYVALLTKVDDCSEVLQDNFLNMSRSMTSQSRVMNVHKMLGIPISNILMVGNYASDLELDPMKDILILSALRQMLRAADDFLEDLPLEETGAIERALQPCI

SEQ ID NO: 41 - DNA, 2961 bp, Homo sapiens
aagagatgatttctccatcctgaacgtgcagcgagcttgtcaggaagatcggaggtgccaagtagcagagaaagcatcccccagctctgacagggagacagcacatgtctaaggcccacaagccttggccctaccggaggagaagtcaattttcttctcgaaaatacctgaaaaaagaaatgaattccttccagcaacagccaccgccattcggcacagtgccaccacaaatgatgtttcctccaaactggcagggggcagagaaggacgctgctttcctcgccaaggacttcaactttctcactttgaacaatcagccaccaccaggaaacaggagccaaccaagggcaatggggcccgagaacaacctgtacagccagtacgagcagaaggtgcgcccctgcattgacctcatcgactccctgcgggctctgggtgtggagcaggacctggccctgccagccatcgccgtcatcggggaccagagctcgggcaagagctctgtgctggaggcactgtcaggagtcgcgcttcccagaggcagcggaatcgtaaccaggtgtccgctggtgctgaaactgaaaaagcagccctgtgaggcatgggccggaaggatcagctaccggaacaccgagctagagcttcaggaccctggccaggtggagaaagagatacacaaagcccagaacgtcatggccgggaatggccggggcatcagccatgagctcatcagcctggagatcacctcccctgaggttccagacctgaccatcattgaccttcccggcatcaccagggtggctgtggacaaccagccccgagacatcggactgcagatcaaggctctcatcaagaagtacatccagaggcagcagacgatcaacttggtggtggttccctgtaacgtggacattgccaccacggaggcgctgagcatggcccatgaggtggacccggaaggggacaggaccatcggtatcctgaccaaaccagatctaatggacaggggcactgagaaaagcgtcatgaatgtggtgcggaacctcacgtaccccctcaagaagggctacatgattgtgaagtgccggggccagcaggagatcacaaacaggctgagcttggcagaggcaaccaagaaagaaattacattctttcaaacacatccatatttcagagttctcctggaggaggggtcagccacggttccccgactggcagaaagacttaccactgaactcatcatgcatatccaaaaatcgctcccgttgttagaaggacaaataagggagagccaccagaaggcgaccgaggagctgcggcgttgcggggctgacatccccagccaggaggccgacaagatgttctttctaattgagaaaatcaagatgtttaatcaggacatcgaaaagttagtagaaggagaagaagttgtaagggagaatgagacccgtttatacaacaaaatcagagaggattttaaaaactgggtaggcatacttgcaactaatacccaaaaagttaaaaatattatccacgaagaagttgaaaaatatgaaaagcagtatcgaggcaaggagcttctgggatttgtcaactacaagacatttgagatcatcgtgcatcagtacatccagcagctggtggagcccgcccttagcatgctccagaaagccatggaaattatccagcaagctttcattaacgtggccaaaaaacattttggcgaatttttcaaccttaaccaaactgttcagagcacgattgaagacataaaagtgaaacacacagcaaaggcagaaaacatgatccaacttcagttcagaatggagcagatggttttttgtcaagatcagatttacagtgttgttctgaagaaagtccgagaagagatttttaaccctctggggacgccttcacagaatatgaagttgaactctcattttcccagtaatgagtcttcggtttcctcctttactgaaataggcatccacctgaatgcctacttcttggaaaccagcaaacgtctcgccaaccagatcccatttataattcagtattttatgctccgagagaatggtgactccttgcagaaagccatgatgcagatactacaggaaaaaaatcgctattcctggctgcttcaagagcagagtgagaccgctaccaagagaagaatccttaaggagagaatttaccggctcactcaggcgcgacacgcactctgtcaattctccagcaaagagatccactgaagggcggcgatgcctgtggttgttttcttgtgcgtactcattcattctaaggggagtcggtgcaggatgccgcttctgctttggggccaaactcttctgtcactatcagtgtccatctctactgtactccctcagcatcagagcatgcatcaggggtccacacaggctcagctctctccaccacccagctcttccctgaccttcacgaagggatggctctccagtccttgggtcccgtagcacacagttacagtgtcctaagatactgctatcattcttcgctaatttgtatttgtattcccttccccctacaagattatgagaccccagagggggaaggtctgggtcaaattcttcttttgtatgtccagtctcctgcacagcacctgcagcattgtaactgcttaataaatgacatctcactgaacgaatgagtgctgtgtaagtgatggagatacctgaggctattgctcaagcccaggccttggacatttagtgactgttagccggtccctttcagatccagtggccatgccccctgcttcccatggttcactgtcattgtgtttcccagcctctccactcccccgccagaaaggagcctgagtgattctcttttcttcttgtttccctgattatgatgagcttccattgttctgttaagtcttgaagaggaatttaataaagcaaagaaactttttaaaaacgt

SEQ ID NO: 42 - protein, 715 aa, Homo sapiens
MSKAHKPWPYRRRSQFSSRKYLKKEMNSFQQQPPPFGTVPPQMMFPPNWQGAEKDAAFLAKDFNFLTLNNQPPPGNRSQPRAMGPENNLYSQYEQKVRPCIDLIDSLRALGVEQDLALPAIAVIGDQSSGKSSVLEALSGVALPRGSGIVTRCPLVLKLKKQPCEAWAGRISYRNTELELQDPGQVEKEIHKAQNVMAGNGRGISHELISLEITSPEVPDLTIIDLPGITRVAVDNQPRDIGLQIKALIKKYIQRQQTINLVVVPCNVDIATTEALSMAHEVDPEGDRTIGILTKPDLMDRGTEKSVMNVVRNLTYPLKKGYMIVKCRGQQEITNRLSLAEATKKEITFFQTHPYFRVLLEEGSATVPRLAERLTTELIMHIQKSLPLLEGQIRESHQKATEELRRCGADIPSQEADKMFFLIEKIKMFNQDIEKLVEGEEVVRENETRLYNKIREDFKNWVGILATNTQKVKNIIHEEVEKYEKQYRGKELLGFVNYKTFEIIVHQYIQQLVEPALSMLQKAMEIIQQAFINVAKKHFGEFFNLNQTVQSTIEDIKVKHTAKAENMIQLQFRMEQMVFCQDQIYSVVLKKVREEIFNPLGTPSQNMKLNSHFPSNESSVSSFTEIGIHLNAYFLETSKRLANQIPFIIQYFMLRENGDSLQKAMMQILQEKNRYSWLLQEQSETATKRRILKERIYRLTQARHALCQFSSKEIH

SEQ ID NO: 43 - DNA, 3512 bp, Homo sapiens
aactcagctgagtgttagtcaaagaaggtgtgtcctgctccccaatgacaggttgctcagagactgctgatttccatccctatataaagagagtccctggcatacagagactgctctgctccaggcatctgccacaatgtgggtgcttacacctgctgcttttgctgggaagctcttgagtgtgttcaggcaacctctgagctctctgtggaggagcctggtcccgctgttctgctggctgagggcaaccttctggctgctagctaccaagaggagaaagcagcagctggtcctgagagggccagatgagaccaaagaggaggaagaggaccctcctctgcccaccaccccaaccagcgtcaactatcacttcactcgccagtgcaactacaaatgcggcttctgtttccacacagccaaaacatcctttgtgctgccccttgaggaagcaaagagaggattgcttttgcttaaggaagctggtatggagaagatcaacttttcaggtggagagccatttcttcaagaccggggagaatacctgggcaagttggtgaggttctgcaaagtagagttgcggctgcccagcgtgagcatcgtgagcaatggaagcctgatccgggagaggtggttccagaattatggtgagtatttggacattctcgctatctcctgtgacagctttgacgaggaagtcaatgtccttattggccgtggccaaggaaagaagaaccatgtggaaaaccttcaaaagctgaggaggtggtgtagggattatagagtcgctttcaagataaattctgtcattaatcgtttcaacgtggaagaggacatgacggaacagatcaaagcactaaaccctgtccgctggaaagtgttccagtgcctcttaattgagggtgagaattgtggagaagatgctctaagagaagcagaaagatttgttattggtgatgaagaatttgaaagattcttggagcgccacaaagaagtgtcctgcttggtgcctgaatctaaccagaagatgaaagactcctaccttattctggatgaatatatgcgctttctgaactgtagaaagggacggaaggacccttccaagtccatcctggatgttggtgtagaagaagctataaaattcagtggatttgatgaaaagatgtttctgaagcgaggaggaaaatacatatggagtaaggctgatctgaagctggattggtagagcggaaagtggaacgagacttcaacacaccagtgggaaaactcctagagtaactgccattgtctgcaatactatcccgttggtatttcccagtggctgaaaacctgattttctgctgcacgtggcatctgattacctgtggtcactgaacacacgaataacttggatagcaaatcctgagacaatggaaaaccattaactttacttcattggcttataaccttgttgttattgaaacagcacttctgtttttgagtttgttttagctaaaaagaaggaatacacacaggaataatgaccccaaaaatgcttagataaggcccctatacacaggacctgacatttagctcaatgatgcgtttgtaagaaataagctctagtgatatctgtgggggcaaaatttaatttggatttgattttttaaaacaatgtttactgcgatttctatatttccattttgaaactatttcttgttccaggtttgttcatttgacagagtcagtattttttgccaaatatccagataaccagttttcacatctgagacattacaaagtatctgcctcaattatttctgctggttataatgcttttttttttttgcctttatgccattgcagtcttgtactttttactgtgatgtacagaaatagtcaacagatgtttccaagaacatatgatatgataatcctaccaattttcaagaagtctctagaaagagataacacatggaaagacggtgtggtgcagcccagcccacggtggctgttccatgaatgctggctacctatgtgtgtggtacctgttgtgtccctttctcttcaaagatcctgagcaaaacaaagatacgctttccatttgatgatggagttgacatggaggcagtgcttgcattgctttgttcgcctatcatctggccacatgaggctgtcaagcaaaagaataggagtgtagttgagtagctggttggccctacatctctgagaagtgacggcacactgggttggcataagatatcctaaaatcacgctggaaccttgggcaaggaagaatgtgagcaagagtagagagagtgcctggatttcatgtcagtgaagccaagtcaccatatcatatttttgaatgaactctgagtcagttgaaatagggtaccatctaggtcagtttaagaagagtcagctcagagaaagcaagcataagggaaaatgtcacgtaaactagatcagggaacaaaatcctctccttgtggaaatatcccatgcagtttgttgatacaacttagtatcttattgcctaaaaaaaaatttcttatcattgtttcaaaaaagcaaaatcatggaaaatttttgttgtccaggcaaataaaaggtcattttaatttagctgcaatttcagtgttcctcactaggtggcatttaaatgtcgcctgatgtcattaagcaccatccaaaaagtctgcttcataatctattttcaagacttggtgattctgaaagttttggtttttgtgactttgtttctcaggaaaaaaaatattcctacttaaattttaagtctataattcaatttaaatatgtgtgtgtctcatccaggataggataggttgtcttctattttccattttacctatttactttttttgtaagaaaagagaaaaatgaattctaaagatgttccccatgggttttgattgtgtctaagctatgatgaccttcatataatcagcataaacataaaacaaattttttacttaacatgagtgcactttactaatcctcatggcacagtggctcacgcctgtaatcccagcacttgggaggacaatgtgggtggatcacgaggtcaggagttcgagaacagcctggccaacatggtgaaaccccgtctccactaaaaatacaaaaattagccaggcatggtggcgtacacttgtaattccagctactcaagaggctgaggcaggaggattgcttgaaccctgaaggcagaggttacagagccaagatagcgccactgcactccagcctggatgacagagcaagactccgtctcaaaaaaaaaaaaaaaaaaaagcaagagagttcaactaagaaaggtcacatatgtgaaagcccaaggacactgtttgatatacagcaggtattcaatcagtgttatttgaaaccaaatctgaatttgaagtttgaatcttctgagttggaatgaatttttttctagctgagggaaactgtatttttctttccccaaagaggaatgtaatgtaaagtgaaataaaactataagctatgtt

SEQ ID NO: 44 - protein, 361 aa, Homo sapiens
MWVLTPAAFAGKLLSVFRQPLSSLWRSLVPLFCWLRATFWLLATKRRKQQLVLRGPDETKEEEEDPPLPTTPTSVNYHFTRQCNYKCGFCFHTAKTSFVLPLEEAKRGLLLLKEAGMEKINFSGGEPFLQDRGEYLGKLVRFCKVELRLPSVSIVSNGSLIRERWFQNYGEYLDILAISCDSFDEEVNVLIGRGQGKKNHVENLQKLRRWCRDYRVAFKINSVINRFNVEEDMTEQIKALNPVRWKVFQCLLIEGENCGEDALREAERFVIGDEEFERFLERHKEVSCLVPESNQKMKDSYLILDEYMRFLNCRKGRKDPSKSILDVGVEEAIKFSGFDEKMFLKRGGKYIWSKADLKLDW

SEQ ID NO: 45 - DNA, 4034 bp, Homo sapiens
agtttctgagcgctcggcatctgattcaatctccagtttcctgttcttgctggggctggggtctctcctttaacaaagacacgccgcgcggccgagtccaggggctgcagaggcctggcgcgcgcacgcgcacgcgcacgcccaccgcgcggcttcccgcggtccccggtgctgaggagagagcgatccgagggactgcgccgcccggacggcctgcagagcgctgccatcatgagtgaaattcgtaaggacaccttgaaggccattctgttggagttagaatgtcattttacatggaatttacttaaggaagacattgatctgtttgaggtagaagatacaattgggcaacagcttgaatttcttaccacaaaatctagacttgctctttataacctattggcctatgtgaaacacctaaaaggccaaaataaagacgcccttgagtgcttggaacaagcagaagaaataatccagcaagaacactcagacaaagaagaagtacgaagcctggtcacttggggaaactatgcctgggtgtattatcacatggaccagcttgaagaagctcagaagtatacaggtaagatagggaatgtctgtaagaaattgtccagtccttctaactacaagttggagtgtcctgagactgactgtgagaaaggctgggcactcttgaaatttggaggaaagtattatcaaaaggctaaagcggcttttgagaaggctctggaagtggagcctgacaatccagaatttaacatcggctatgctatcacagtgtatcggctggatgattctgatagagaagggtctgtaaagagcttttctctggggcctttgagaaaggctgttaccctgaacccagataacagctatattaaggtttttctggcactgaagcttcaagatgtacatgcagaagctgaaggggaaaagtatattgaagaaatcctggaccaaatatcatcccagccttacgtccttcgttatgcagccaagttctataggagaaaaaattcctggaacaaagctctcgaacttttaaaaaaggccttggaggtgacaccaacttcttctttcctgcatcaccagatgggactttgctacagggcacaaatgatccaaatcaagaaggccacacacaacagacctaaaggaaaggataaactaaaggttgatgagctgatttcatctgctatatttcatttcaaagcagccatggaacgagactctatgtttgcatttgcctacacagacctggccaacatgtacgctgaaggaggccagtatagcaatgctgaggacattttccggaaagctcttcgtctggagaacataaccgatgatcacaaacatcagatccattaccactatggccgctttcaggaatttcaccgtaaatcagaaaatactgccatccatcattatttagaagccttaaaggtcaaagacagatcaccccttcgcaccaaactgacaagtgctctgaagaaattgtctaccaagagactttgtcacaatgctttagatgtgcagagtttaagtgccctagggtttgtttacaagctggaaggagaaaagaggcaagctgctgagtactatgagaaggcacaaaagatagatccagaaaatgcagaattcctgactgctctctgtgagctccgactttccatttaaatacatactctaggaaattagctctaagtttttcccttcattttgggttctcctgtttgttttttttttattattttaatcccttgtttattatagagctaatatttattgaatagttattgtgtaccaagcattgtgctaaatactttatatgcattatgatgaatcttgtgcggttttctttctttttttctttttaattaaaatactataatccattgagaaatagcaatattctagctattgtaacttctaaaaatggtatggccattagatctgtgctttttatctctgctctttgaatttctcatattatatagtaaatatattcctacgtaaacctttgatacctagatcaggaatactcttccaggagtacaaaattacattattgatagttaagctcttaattgtgtagcttgcaaaagacagcactttttagttacagatgttttgactttgatgaggatatttagctatcaatctaatagtcacctaaaatatcttttttgttggaaaaaagtttataataaaaaagtttgtcatctctagtgacttcaataaagaaaaaactagaagaggagaaaaaggatttcctcaaattttaaatatgtaacttcagggattcaatccccaaatgtttattaagtagctagaaataattatgtggaaaaaaatgaataatggaaaatagtgagtctcaaattgttctctttttttttttaactaaaacaaatctgcaatgaatctagatgcaattaattttattccttccaactaaaattacaatatttttaggttaaaattattgagatataaagcagccattgggaaattgggagaaatgataaacaaatggaaaaagaagatgtccctaacctacacccatagattaccaaggtttcagtgtactagttttgaatctgttctgaatggagtttttataccctcaatttctggcctttggctattttagcatttcaaagtgacttctatgaagctttttttttaatgtgaaattttcagaatgttgtttttttcatgtagatactccaggaagagttaagcactgctttcagttttaatatccaccttgaggggtcgctgcttgagggctcttatcccaggggactttttaattcggatgttacttaatgtggcttctctaatgtagtttctttgattaccgactacacaattatgtaccatcacagtattagtggaaaagtaccatgtgatttaattctccattcctccaatgtaactcttaaaattattatgtatgtgtgtgtgttttactttttgttttttatcatctttaaaatttctattatggtttgattattataaaaataatgaattctcactgtaaatttcaaaaaaaaattacaaaagtatgtgaatttaaaaatgagagcagtcctctcaccctaccacagttccacaccctcaaggtaaacttataacttataatttgatatgtaaacttccagatcttttttctatgcgtaatcagacatacatatatactgcagtgtatctcacgtattaatttttaaaaatcttttgttttacttaattctgtttttattattattattattttgtttgatctattaaggaagaacaaggaagggaatgatctttactcaagaatttcagaaagtcagcactgaagtcctgacctatcagtagacacatttgtccctttcagatattttaggatattctagcaaagcaggccatttctcccacctgaaagtacataacttctatcacttgccacataattaaaagaactcacattaagcggttactcagacagttaatcatagaaaagattatttgcttcatcagttcatagaaaagattatttgcttcatcagttaacttgtttttataaatcagggctgtgttcatacacagaaggggcctgagatttctgcactttaaacaagctcctcctaggtgaggatgctgtggctgttctaattacattttgagtagtaaggtctacagcattgttcctcaaacttggctacgtattggaatcacctaaaaagttaaaacaaaacatggatgtctgggtcccgccccatagagaatgacttaattggcatggggtgcagtccaggcatcatgatttttagatttcccagttggaacttgtgcagcaaagtttgggagctactgatggacatgtgaaaagtaagtataaatggaataaaattaattaggctaataggcttaacccaggaaatcctaagttccttgaatatccagtttgcatttggactcctcatcatatacttggtatataatactctaataaaagctgcctgagttgaattgta

SEQ ID NO: 46 - protein, 482 aa, Homo sapiens
MSEIRKDTLKAILLELECHFTWNLLKEDIDLFEVEDTIGQQLEFLTTKSRLALYNLLAYVKHLKGQNKDALECLEQAEEIIQQEHSDKEEVRSLVTWGNYAWVYYHMDQLEEAQKYTGKIGNVCKKLSSPSNYKLECPETDCEKGWALLKFGGKYYQKAKAAFEKALEVEPDNPEFNIGYAITVYRLDDSDREGSVKSFSLGPLRKAVTLNPDNSYIKVFLALKLQDVHAEAEGEKYIEEILDQISSQPYVLRYAAKFYRRKNSWNKALELLKKALEVTPTSSFLHHQMGLCYRAQMIQIKKATHNRPKGKDKLKVDELISSAIFHFKAAMERDSMFAFAYTDLANMYAEGGQYSNAEDIFRKALRLENITDDHKHQIHYHYGRFQEFHRKSENTAIHHYLEALKVKDRSPLRTKLTSALKKLSTKRLCHNALDVQSLSALGFVYKLEGEKRQAAEYYEKAQKIDPENAEFLTALCELRLSI

SEQ ID NO: 47 - DNA, 733 bp, Homo sapiens
aaacagcaggaaatagaaacttaagagaaatacacacttctgagaaactgaaacgacaggggaaaggaggtctcactgagcaccgtcccagcatccggacaccacagcggcccttcgctccacgcagaaaaccacacttctcaaaccttcactcaacacttccttccccaaagccagaagatgcacaaggaggaacatgaggtggctgtgctgggggcaccccccagcaccatccttccaaggtccaccgtgatcaacatccacagcgagacctccgtgcccgaccatgtcgtctggtccctgttcaacaccctcttcttgaactggtgctgtctgggcttcatagcattcgcctactccgtgaagtctagggacaggaagatggttggcgacgtgaccggggcccaggcctatgcctccaccgccaagtgcctgaacatctgggccctgattctgggcatcctcatgaccattggattcatcctgttactggtattcggctctgtgacagtctaccatattatgttacagataatacaggaaaaacggggttactagtagccgcccatagcctgcaacctttgcactccactgtgcaatgctggccctgcacgctggggctgttgcccctgcccccttggtcctgcccctagatacagcagtttatacccacacacctgtctacagtgtcattcaataaagtgcacgtgcttgtgaaaaaaaaaaaaaaaaa

SEQ ID NO: 48 - protein, 125 aa, Homo sapiens
MHKEEHEVAVLGAPPSTILPRSTVINIHSETSVPDHVVWSLFNTLFLNWCCLGFIAFAYSVKSRDRKMVGDVTGAQAYASTAKCLNIWALILGILMTIGFILLLVFGSVTVYHIMLQIIQEKRGY

SEQ ID NO: 49 - DNA, 678 bp, Homo sapiens
aggaaaaggaaactgttgagaaaccgaaactactggggaaagggagggctcactgagaaccatcccagtaacccgaccgccgctggtcttcgctggacaccatgaatcacactgtccaaaccttcttctctcctgtcaacagtggccagccccccaactatgagatgctcaaggaggagcacgaggtggctgtgctgggggcgccccacaaccctgctcccccgacgtccaccgtgatccacatccgcagcgagacctccgtgcccgaccatgtcgtctggtccctgttcaacaccctcttcatgaacccctgctgcctgggcttcatagcattcgcctactccgtgaagtctagggacaggaagatggttggcgacgtgaccggggcccaggcctatgcctccaccgccaagtgcctgaacatctgggccctgattctgggcatcctcatgaccattctgctcatcgtcatcccagtgctgatcttccaggcctatggatagatcaggaggcatcactgaggccaggagctctgcccatgacctgtatcccacgtactccaacttccattcctcgccctgcccccggagccgagtcctgtatcagccctttatcctcacacgcttttctacaatggcattcaataaagtgcacgtgtttctggtgctaaaaaaaaaa

SEQ ID NO: 50 - protein, 133 aa, Homo sapiens
MNHTVQTFFSPVNSGQPPNYEMLKEEHEVAVLGAPHNPAPPTSTVIHIRSETSVPDHVVWSLFNTLFMNPCCLGFIAFAYSVKSRDRKMVGDVTGAQAYASTAKCLNIWALILGILMTILLIVIPVLIFQAYG

SEQ ID NO: 51 - DNA, 1972 bp, Homo sapiens
gaaactcccgcctggccaccataaaagcgccggccctccgcttccccgcgagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcccagggctccctgctggggagctgtacgggtgggcagtagagacgacccccagccccgggccccagcccgcggcactaacgacaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 52 - protein, 503 aa, Homo sapiens
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGPGLPAGELYGWAVETTPSPGPQPAALTTGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 53 - DNA, 1885 bp, Homo sapiens
gaaactcccgcctggccaccataaaagcgccggccctccgcttccccgcgagacgaaacttcccgtcccggcggctctggcacccagggtccggcctgcgccttcccgccaggcctggacactggttcaacacctgtgacttcatgtgtgcgcgccggccacacctgcagtcacacctgtagccccctctgccaagagatccataccgaggcagcgtcggtggctacaagccctcagtccacacctgtggacacctgtgacacctggccacacgacctgtggccgcggcctggcgtctgctgcgacaggagcccttacctcccctgttataacacctgaccgccacctaactgcccctgcagaaggagcaatggccttggctcctgagagggcagccccacgcgtgctgttcggagagtggctccttggagagatcagcagcggctgctatgaggggctgcagtggctggacgaggcccgcacctgtttccgcgtgccctggaagcacttcgcgcgcaaggacctgagcgaggccgacgcgcgcatcttcaaggcctgggctgtggcccgcggcaggtggccgcctagcagcaggggaggtggcccgccccccgaggctgagactgcggagcgcgccggctggaaaaccaacttccgctgcgcactgcgcagcacgcgtcgcttcgtgatgctgcgggataactcgggggacccggccgacccgcacaaggtgtacgcgctcagccgggagctgtgctggcgagaaggcccaggcacggaccagactgaggcagaggcccccgcagctgtcccaccaccacagggtgggcccccagggccattcctggcacacacacatgctggactccaagccccaggccccctccctgccccagctggtgacaagggggacctcctgctccaggcagtgcaacagagctgcctggcagaccatctgctgacagcgtcatggggggcagatccagtcccaaccaaggctcctggagagggacaagaagggcttcccctgactggggcctgtgctggaggcgaggccgcggccccagagtccccgcaccaggcagagccgtacctgtcaccctccccaagcgcctgcaccgcggtgcaagagcccagcccaggggcgctggacgtgaccatcatgtacaagggccgcacggtgctgcagaaggtggtgggacacccgagctgcacgttcctatacggccccccagacccagctgtccgggccacagacccccagcaggtagcattccccagccctgccgagctcccggaccagaagcagctgcgctacacggaggaactgctgcggcacgtggcccctgggttgcacctggagcttcgggggccacagctgtgggcccggcgcatgggcaagtgcaaggtgtactgggaggtgggcggacccccaggctccgccagcccctccaccccagcctgcctgctgcctcggaactgtgacacccccatcttcgacttcagagtcttcttccaagagctggtggaattccgggcacggcagcgccgtggctccccacgctataccatctacctgggcttcgggcaggacctgtcagctgggaggcccaaggagaagagcctggtcctggtgaagctggaaccctggctgtgccgagtgcacctagagggcacgcagcgtgagggtgtgtcttccctggatagcagcagcctcagcctctgcctgtccagcgccaacagcctctatgacgacatcgagtgcttccttatggagctggagcagcccgcctagaacccagtctaatgagaactccagaaagctggagcagcccacctagagctggccgcggccgcccagtctaataaaaagaactccagaaca

SEQ ID NO: 54 - protein, 474 aa, Homo sapiens
MALAPERAAPRVLFGEWLLGEISSGCYEGLQWLDEARTCFRVPWKHFARKDLSEADARIFKAWAVARGRWPPSSRGGGPPPEAETAERAGWKTNFRCALRSTRRFVMLRDNSGDPADPHKVYALSRELCWREGPGTDQTEAEAPAAVPPPQGGPPGPFLAHTHAGLQAPGPLPAPAGDKGDLLLQAVQQSCLADHLLTASWGADPVPTKAPGEGQEGLPLTGACAGGEAAAPESPHQAEPYLSPSPSACTAVQEPSPGALDVTIMYKGRTVLQKVVGHPSCTFLYGPPDPAVRATDPQQVAFPSPAELPDQKQLRYTEELLRHVAPGLHLELRGPQLWARRMGKCKVYWEVGGPPGSASPSTPACLLPRNCDTPIFDFRVFFQELVEFRARQRRGSPRYTIYLGFGQDLSAGRPKEKSLVLVKLEPWLCRVHLEGTQREGVSSLDSSSLSLCLSSANSLYDDIECFLMELEQPA

SEQ ID NO: 55 - DNA, 685 bp, Homo sapiens
ataatagggccggtgctgcctgccgaagccggcggctgagaggcagcgaactcatctttgccagtacaggagcttgtgccgtggcccacagcccacagcccacagccatgggctgggacctgacggtgaagatgctggcgggcaacgaattccaggtgtccctgagcagctccatgtcggtgtcagagctgaaggcgcagatcacccagaagatcggcgtgcacgccttccagcagcgtctggctgtccacccgagcggtgtggcgctgcaggacagggtcccccttgccagccagggcctgggccccggcagcacggtcctgctggtggtggacaaatgcgacgaacctctgagcatcctggtgaggaataacaagggccgcagcagcacctacgaggtacggctgacgcagaccgtggcccacctgaagcagcaagtgagcgggctggagggtgtgcaggacgacctgttctggctgaccttcgaggggaagcccctggaggaccagctcccgctgggggagtacggcctcaagcccctgagcaccgtgttcatgaatctgcgcctgcggggaggcggcacagagcctggcgggcggagctaagggcctccaccagcatccgagcaggatcaagggccggaaataaaggctgttgtaaagagaaaaaaaaaaaaaaaaaaaaa

SEQ ID NO: 56 - protein, 165 aa, Homo sapiens
MGWDLTVKMLAGNEFQVSLSSSMSVSELKAQITQKIGVHAFQQRLAVHPSGVALQDRVPLASQGLGPGSTVLLVVDKCDEPLSILVRNNKGRSSTYEVRLTQTVAHLKQQVSGLEGVQDDLFWLTFEGKPLEDQLPLGEYGLKPLSTVFMNLRLRGGGTEPGGRS

SEQ ID NO: 57 - DNA, 665 bp, Homo sapiens
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggccatggcggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 58 - protein, 122 aa, Homo sapiens
MEASALTSSAVTSVAKVVRVASGSAVVLPLARIATVVIGGVVAMAAVPMVLSAMGFTAAGIASSSIAAKMMSAAAIANGGGVASGSLVATLQSLGATGLSGLTKFILGSIGSAIAAVIARFY

SEQ ID NO: 59 - DNA, 656 bp, Homo sapiens
gggaacacatccaagcttaagacggtgaggtcagcttcacattctcaggaactctccttctttgggtctggctgaagttgaggatctcttactctctaggccacggaattaacccgagcaggcatggaggcctctgctctcacctcatcagcagtgaccagtgtggccaaagtggtcagggtggcctctggctctgccgtagttttgcccctggccaggattgctacagttgtgattggaggagttgtggctgtgcccatggtgctcagtgccatgggcttcactgcggcgggaatcgcctcgtcctccatagcagccaagatgatgtccgcggcggccattgccaatgggggtggagttgcctcgggcagccttgtggctactctgcagtcactgggagcaactggactctccggattgaccaagttcatcctgggctccattgggtctgccattgcggctgtcattgcgaggttctactagctccctgcccctcgccctgcagagaagagaaccatgccaggggagaaggcacccagccatcctgacccagcgaggagccaactatcccaaatatacctggggtgaaatataccaaattctgcatctccagaggaaaataagaaataaagatgaattgttgcaactcttcaaaa

SEQ ID NO: 60 - protein, 119 aa, Homo sapiens
MEASALTSSAVTSVAKVVRVASGSAVVLPLARIATVVIGGVVAVPMVLSAMGFTAAGIASSSIAAKMMSAAAIANGGGVASGSLVATLQSLGATGLSGLTKFILGSIGSAIAAVIARFY

SEQ ID NO: 61 - DNA, 8040 bp, Homo sapiens
ctcctccccggcgcgctccctgcccctcgctccccgcagccagcagagaaggcggaagcagtggcgtccgcagctggggcttggcctgcgggcggccagcgaaggtggcgaaggctcccactggatccagagtttgccgtccaagcagcctcgtctcggcgcgcagtgtctgtgtccgtcctctaccagcgccttggctgagcggagtcgtgcggttggtgggggagccctgccctcctggttcggcctccccgcgcactagaacgatcatgaacttctgaagggacccagctttctttgtgtgctccaagtgatttgcacaaataataatatatatatttattgaaggagagaatcagagcaagtgataatcaagttactatgagtctgctaaactgtgaaaacagctgtggatccagccagtctgaaagtgactgctgtgtggccatggccagctcctgtagcgctgtaacaaaagatgatagtgtgggtggaactgccagcacggggaacctctccagctcatttatggaggagatccagggatatgatgtagagtttgacccacccctggaaagcaagtatgaatgccccatctgcttgatggcattacgagaagcagtgcaaacgccatgcggccataggttctgcaaagcctgcatcataaaatcaataagggatgcaggtcacaaatgtccagttgacaatgaaatactgctggaaaatcaactatttccagacaattttgcaaaacgtgagattctttctctgatggtgaaatgtccaaatgaaggttgtttgcacaagatggaactgagacatcttgaggatcatcaagcacattgtgagtttgctcttatggattgtccccaatgccagcgtcccttccaaaaattccatattaatattcacattctgaaggattgtccaaggagacaggtttcttgtgacaactgtgctgcatcaatggcatttgaagataaagagatccatgaccagaactgtcctttggcaaatgtcatctgtgaatactgcaatactatactcatcagagaacagatgcctaatcattatgatctagactgccctacagccccaattccatgcacattcagtacttttggttgccatgaaaagatgcagaggaatcacttggcacgccacctacaagagaacacccagtcacacatgagaatgttggcccaggctgttcatagtttgagcgttatacccgactctgggtatatctcagaggtccggaatttccaggaaactattcaccagttagagggtcgccttgtaagacaagaccatcaaatccgggagctgactgctaaaatggaaactcagagtatgtatgtaagtgagctcaaacgaaccattcgaacccttgaggacaaagttgctgaaatcgaagcacagcagtgcaatggaatttatatttggaagattggcaactttggaatgcatttgaaatgtcaagaagaggagaaacctgttgtgattcatagccctggattctacactggcaaacccgggtacaaactgtgcatgcgcttgcaccttcagttaccgactgctcagcgctgtgcaaactatatatccctttttgtccacacaatgcaaggagaatatgacagccacctcccttggcccttccagggtacaatacgccttacaattcttgatcagtctgaagcacctgtaaggcaaaaccacgaagagataatggatgccaaaccagagctgcttgctttccagcgacccacaatcccacggaacccaaaaggttttggctatgtaacttttatgcatctggaagccctaagacaaagaactttcattaaggatgacacattattagtgcgctgtgaggtctccacccgctttgacatgggtagccttcggagggagggttttcagccacgaagtactgatgcaggggtatagcttgccctcacttgctcaaaaacaactacctggagaaaacagtgcctttccttgccctgttctcaataacatgcaaacaaacaagccacgggaaatatgtaatatctactagtgagtgttgttagagaggtcacttactatttcttcctgttacaaatgatctgaggcagttttttcctgggaatccacacgttccatgctttttcagaaatgttaggcctgaagtgcctgtggcatgttgcagcagctattttgccagttagtatacctctttgttgtactttcttgggcttttgctctggtgtattttattgtcagaaagtccagactcaagagtactaaacttttaataataatggattttccttaaaacttcagtctttttgtagtattatatgtaatatattaaaagtgaaaatcactaccgccttgtgctagtgccctcgagaagagttattgctctagaaagttgagttctcatttttttaacctgttatagatttcagaggatttgaaccataatccttggaaaacttaagttctcattcaccccagtttttcctccaggttgttactaaggatattcagggatgagtttaaaccctaaatataaccttaattatttagtgtaaacatgtctgttgaataatacttgtttaagtgttccttctgccttgcttacttatttccttgaggttacgaagtagcatcttccccagagtttataatgctgagaaccacgtggataccaactgctcattgttatgctatgtaaccctttttgtctattcagtgcagagtgaatttcacagctctgcatatgtcttcatttgtttaatgcttacaagacaggagatgcacacatacaatcagcaacataaaaattaaaagtgacccaagtagtcagcgcatgtggcatctcattggtggtgacagaagctatgtgagccagaagttttcagctcttttgaataccctctggtttatttcgattaaaaagaacaaaattgatttcctaaaatcagaattttttaaaacttgggagatgattggagatacctaggaggtcaccaaactaggattagaagtcacagtggttgtatcacaacttagcttgagtatgttgctgtagcctaacaactgcaggttctgagaaggatcctgtagaatcctggaagtaaccagattttcctaatagggagatgatttttttgtgtgccatcatgtatttgttaaaggcctatatatagatataaaatatcgtggaatctagttctcagggagacccgcaactagtataagcttataaaggatctaaagatccatccaccatttaaagttgtctggtaatgagagatgacattgtatcccccagagaggccaaatcagagtcgccagccagcgttctagatcagccttaatttcaagagaaagccaaggacctcatctgcaggggagtgtggttttcagccccagcgagtgtcactttgaactttccctttgcttttttctctcttctccctccccacccacccttaggctcctgatctggtgagtttgttatggagtgaaaataaaagtcaagcagagaccttgtttcccgtgccaccattagtaccacaagctcatggctagttaccacattacttcctggcagtttgtgtccctcagctgtgccttccaaccagcgcctgagaatcactgcataccaccctctaggtagggaaacctacactgctgctgttcctgtgattattttacaatgaataaataattgtcaagttccatttaaaaactgaacagtagtatttttgtatttgcgtagaaaaagcctgaaggaaatatactaaactttttgttggcttattttcctttgcgcttgcttatattttttacattttctacaataaatgtgtacttttatcggagaaaaaaattaaatgttgccacaaaacatttaatctccacgcccccagctcaaaaaaggaaatgatatttaaaagcttcctggtcagatttctattaaaagcactggctgtgcattagatacaaagaggagtcatttcctgccttggtgatactatttttttctactaactcaagagtctttattaaaaaaaaaagttgttttgcctaatttcagcttttagcaagcttcccatctgtaaaatgatttggaccagatatttctagagtcccctccagccataacattctgtctcaaattaagttccaaccagcagaacaatgacaatacttaggaaagtattttgccagtataaaatgtctttaacttactctttgctgacactgatactttcctctaatttagtgtctatcagctgggtcacatcttaagtaaaatgagcaattttaacccccaacatttggcattttgtcataaaccagccagttattttatgctggtcattcatcttgactacaaagtagaatagtcaagctgtcattccaaatagaaaactttttacttcaatcagaattaagccttaacctggaaagttggtttcttccttacattttcccaatctcctactctattcttaaacatgctagtttcactcagttgggtatacaagcctttgggctttatgttgtatgttactaaccaccttttaccatatttatcttttggcatcattctgggacattgctaaattaaaaaagaaattgtttccacttttttctggagatgttcaactaaaggttgttttgttttgttttttgttttgagacagtctcaccctgacgctcaggctggagtgcagtggtgcaacctcggctcactgcaacctccacctcccgggctcaagccattctcctgcctcagcctcccaagcagctgggattacaggcacccgccaccacgcccagctaatttttttgtattttgagtagagaccgggtttcaccatattggccagtctcgtctggaactcctgacctcagatgatccgcccgcctcagcctcccaaagtgctgggattacaggcatgagccaccacgcccagcgtccaacccactgttggatgaaacttgctgcacgtcatacattttgctgttggcaaacaagtctgaatgttgatttgaagtttggtagtttattactatctattggcagcaaagactgtttattggtatactacaatatgatttaacttttattttggggataaatagtagaaaaaagtgaaacagaatgaaggcaggtgttttttattctaatgatggaataatacagagatactggacgatctctagcagttaattattgtgacccatataaaattatacaggtcacagtataattctctattaccgtttttacaccagtaagtcttagataaactaagcatgcttatgaattatgtatacagttagaatgcattatttttacagaggaacaattgcttgtatgtactaacactgttctcttggcttgcctcaagttctactcattattttatataaaatactattaggctgggcacggtggctcacgcctataatcccagcactttgggaggtggaggctggcggattacttgaagccaggagttcgagaccagcctggccaaaatggtgaaaccccatctctataaaaatacaaaaattagccaggtgtcatgatacatgcctgtaatcccagcttcttgggaggctgaggcacgggaatcgcttgaacccgggaagcacaggttgcagtgagccaagatcatgccactgcacccccagcctgggtgacagagtgcaacactgtctcacaaaacaaaacaaaaacatcagattctgtttgtgatgcctagttgcttacaacctaaacagtgcaatgccttaaggaaatgaaaaggagccataagtagtcatttatatttttattttgaagtgtgctttttctaaactcccagattgacatgatggactgtaagttagtttctctgtttctgtctttgtgcctgtagagtgtacttggcacttacaaattcccagtatccagaaagatgatctgatgaaatcaaattggatggatcttggcagactgtgacactcaattacagccttcactttcagtcaaaaacggacacttggcaaggaggtgcctggttgtttcactaaatgtcacttgtgtgtgtaatattttaaagctttttccccacaggaaattcgggtcataaaatcctgaaaaataattctaggtgggaaaagcattttaggaaatgagagatgtggtgctgcttttcttctctcagagtgctttctcagcaggacactagccctgcctttaagatggggaagttggggcatgtgcctctgctcttactgtctgcagctctgaaggtaggtgctgtcccactcggacaatcgcccaagcagcagtgaccatagttctcttctatgcaagtccccaggagaaggtaaactgtgtggaatggggatgtgttctggttgctgctgaatcccctcttcttaccacagtgcctggcacgttgcacacactcaaatacgtaataatgaacatttattgaaagcagcagttgaagctgaccaatttctggtaccttgtcatgtaaattttagatggtaaggcgcagatgttactttttttgctttttttcttcagcacttgatgaaatttcccaaacatgcagaaatgttgaaagacttgtatagtgaacatctacgacctagaatctgcagtaatattatgttacatttgctttatcacttgatagatgttacttttaatgagacttcaagtttggtttctctaaacaaaatattctaaaataactgaacaactttaatcaatttgtcttaagttctttgggggaacttgggacatttgctttgtaactggaattgcagccctcacgttaagctaattttaaactttgcaaatttgttatgctgaatttcagtcttatttattttgcctgaaggggtattttttgtaatggatttatttgaaggtccttgataaattgtgcagaatattctcgtgttctttttgcacttgataaattatctaatttctgtggtgagaatgtaatttggggcctattttgtttatacaagcttccagaattatgttctcagagggatgaaaaggtgtaatttagcatataggtcactaaattaggagctaagacacattttctcctgactgaccatgggtcaatcagttttgtcttcgtgtccttttccttgtaaagtagaaactagaatttgaaatttaaatattaaataatgggtaacattcattaatgtatgactctattaagaaagacactgtgaatccagggaggattctcataattctgtaaactgtatgacaagctgtggaatgaaatctgacttttgaaaattgaaagacatccagtggtcttatcacaaagcctgcttttcctcagaacttaactattgccatggaatttgtaagcagttatcctaatccatctggactctgaaaatgcatcctttatgagagggagtgaatgcaaagataagggtggggaaacactaatcatgaaaagaatgaaaatcagtgttcagttttaagagcaggttgtattgaaggaagggattaaaggaattatccagatttgaggtggcacatcttccaccactccctgcaccatcagcatgcacggagcgcataaaacaagccctgctcctaatggcagtgaaacctcggatggcctccatcaggtcaatacaactgaattgctgggctgacttaagattgaaggactccattttagtaagtagagaagtgtgacctttctcaacccaggttgtgaatgtggattcacacttatctcaaaaaggcacctggagttttaactttatgtcatgtctcagtactggttgcaaggtatgaccaaaagtgttccttgaatggcacctttttgaatattaatttagaagaaaacatgccagactgacatacttaccccctccgcactgttactacttccttaccagccctatgtactgcatcaatgtctacaagaaagcactcttcattaaaatgaaatatatatattaaaataaaaaaaaaaaaaaaaaaaa

SEQ ID NO: 62 - protein, 522 aa, Homo sapiens
MSLLNCENSCGSSQSESDCCVAMASSCSAVTKDDSVGGTASTGNLSSSFMEEIQGYDVEFDPPLESKYECPICLMALREAVQTPCGHRFCKACIIKSIRDAGHKCPVDNEILLENQLFPDNFAKREILSLMVKCPNEGCLHKMELRHLEDHQAHCEFALMDCPQCQRPFQKFHINIHILKDCPRRQVSCDNCAASMAFEDKEIHDQNCPLANVICEYCNTILIREQMPNHYDLDCPTAPIPCTFSTFGCHEKMQRNHLARHLQENTQSHMRMLAQAVHSLSVIPDSGYISEVRNFQETIHQLEGRLVRQDHQIRELTAKMETQSMYVSELKRTIRTLEDKVAEIEAQQCNGIYIWKIGNFGMHLKCQEEEKPVVIHSPGFYTGKPGYKLCMRLHLQLPTAQRCANYISLFVHTMQGEYDSHLPWPFQGTIRLTILDQSEAPVRQNHEEIMDAKPELLAFQRPTIPRNPKGFGYVTFMHLEALRQRTFIKDDTLLVRCEVSTRFDMGSLRREGFQPRSTDAGV

SEQ ID NO: 63 - DNA, 7947 bp, Homo sapiens
ctcctccccggcgcgctccctgcccctcgctccccgcagccagcagagaaggcggaagcagtggcgtccgcagctggggcttggcctgcgggcggccagcgaaggtggcgaaggctcccactggatccagagtttgccgtccaagcagcctcgtctcggcgcgcagtgtctgtgtccgtcctctaccagcgccttggctgagcggagtcgtgcggttggtgggggagccctgccctcctggttcggcctccccgcgcactagaacgagcaagtgataatcaagttactatgagtctgctaaactgtgaaaacagctgtggatccagccagtctgaaagtgactgctgtgtggccatggccagctcctgtagcgctgtaacaaaagatgatagtgtgggtggaactgccagcacggggaacctctccagctcatttatggaggagatccagggatatgatgtagagtttgacccacccctggaaagcaagtatgaatgccccatctgcttgatggcattacgagaagcagtgcaaacgccatgcggccataggttctgcaaagcctgcatcataaaatcaataagggatgcaggtcacaaatgtccagttgacaatgaaatactgctggaaaatcaactatttccagacaattttgcaaaacgtgagattctttctctgatggtgaaatgtccaaatgaaggttgtttgcacaagatggaactgagacatcttgaggatcatcaagcacattgtgagtttgctcttatggattgtccccaatgccagcgtcccttccaaaaattccatattaatattcacattctgaaggattgtccaaggagacaggtttcttgtgacaactgtgctgcatcaatggcatttgaagataaagagatccatgaccagaactgtcctttggcaaatgtcatctgtgaatactgcaatactatactcatcagagaacagatgcctaatcattatgatctagactgccctacagccccaattccatgcacattcagtacttttggttgccatgaaaagatgcagaggaatcacttggcacgccacctacaagagaacacccagtcacacatgagaatgttggcccaggctgttcatagtttgagcgttatacccgactctgggtatatctcagaggtccggaatttccaggaaactattcaccagttagagggtcgccttgtaagacaagaccatcaaatccgggagctgactgctaaaatggaaactcagagtatgtatgtaagtgagctcaaacgaaccattcgaacccttgaggacaaagttgctgaaatcgaagcacagcagtgcaatggaatttatatttggaagattggcaactttggaatgcatttgaaatgtcaagaagaggagaaacctgttgtgattcatagccctggattctacactggcaaacccgggtacaaactgtgcatgcgcttgcaccttcagttaccgactgctcagcgctgtgcaaactatatatccctttttgtccacacaatgcaaggagaatatgacagccacctcccttggcccttccagggtacaatacgccttacaattcttgatcagtctgaagcacctgtaaggcaaaaccacgaagagataatggatgccaaaccagagctgcttgctttccagcgacccacaatcccacggaacccaaaaggttttggctatgtaacttttatgcatctggaagccctaagacaaagaactttcattaaggatgacacattattagtgcgctgtgaggtctccacccgctttgacatgggtagccttcggagggagggttttcagccacgaagtactgatgcaggggtatagcttgccctcacttgctcaaaaacaactacctggagaaaacagtgcctttccttgccctgttctcaataacatgcaaacaaacaagccacgggaaatatgtaatatctactagtgagtgttgttagagaggtcacttactatttcttcctgttacaaatgatctgaggcagttttttcctgggaatccacacgttccatgctttttcagaaatgttaggcctgaagtgcctgtggcatgttgcagcagctattttgccagttagtatacctctttgttgtactttcttgggcttttgctctggtgtattttattgtcagaaagtccagactcaagagtactaaacttttaataataatggattttccttaaaacttcagtctttttgtagtattatatgtaatatattaaaagtgaaaatcactaccgccttgtgctagtgccctcgagaagagttattgctctagaaagttgagttctcatttttttaacctgttatagatttcagaggatttgaaccataatccttggaaaacttaagttctcattcaccccagtttttcctccaggttgttactaaggatattcagggatgagtttaaaccctaaatataaccttaattatttagtgtaaacatgtctgttgaataatacttgtttaagtgttccttctgccttgcttacttatttccttgaggttacgaagtagcatcttccccagagtttataatgctgagaaccacgtggataccaactgctcattgttatgctatgtaaccctttttgtctattcagtgcagagtgaatttcacagctctgcatatgtcttcatttgtttaatgcttacaagacaggagatgcacacatacaatcagcaacataaaaattaaaagtgacccaagtagtcagcgcatgtggcatctcattggtggtgacagaagctatgtgagccagaagttttcagctcttttgaataccctctggtttatttcgattaaaaagaacaaaattgatttcctaaaatcagaattttttaaaacttgggagatgattggagatacctaggaggtcaccaaactaggattagaagtcacagtggttgtatcacaacttagcttgagtatgttgctgtagcctaacaactgcaggttctgagaaggatcctgtagaatcctggaagtaaccagattttcctaatagggagatgatttttttgtgtgccatcatgtatttgttaaaggcctatatatagatataaaatatcgtggaatctagttctcagggagacccgcaactagtataagcttataaaggatctaaagatccatccaccatttaaagttgtctggtaatgagagatgacattgtatcccccagagaggccaaatcagagtcgccagccagcgttctagatcagccttaatttcaagagaaagccaaggacctcatctgcaggggagtgtggttttcagccccagcgagtgtcactttgaactttccctttgcttttttctctcttctccctccccacccacccttaggctcctgatctggtgagtttgttatggagtgaaaataaaagtcaagcagagaccttgtttcccgtgccaccattagtaccacaagctcatggctagttaccacattacttcctggcagtttgtgtccctcagctgtgccttccaaccagcgcctgagaatcactgcataccaccctctaggtagggaaacctacactgctgctgttcctgtgattattttacaatgaataaataattgtcaagttccatttaaaaactgaacagtagtatttttgtatttgcgtagaaaaagcctgaaggaaatatactaaactttttgttggcttattttcctttgcgcttgcttatattttttacattttctacaataaatgtgtacttttatcggagaaaaaaattaaatgttgccacaaaacatttaatctccacgcccccagctcaaaaaaggaaatgatatttaaaagcttcctggtcagatttctattaaaagcactggctgtgcattagatacaaagaggagtcatttcctgccttggtgatactatttttttctactaactcaagagtctttattaaaaaaaaaagttgttttgcctaatttcagcttttagcaagcttcccatctgtaaaatgatttggaccagatatttctagagtcccctccagccataacattctgtctcaaattaagttccaaccagcagaacaatgacaatacttaggaaagtattttgccagtataaaatgtctttaacttactctttgctgacactgatactttcctctaatttagtgtctatcagctgggtcacatcttaagtaaaatgagcaattttaacccccaacatttggcattttgtcataaaccagccagttattttatgctggtcattcatcttgactacaaagtagaatagtcaagctgtcattccaaatagaaaactttttacttcaatcagaattaagccttaacctggaaagttggtttcttccttacattttcccaatctcctactctattcttaaacatgctagtttcactcagttgggtatacaagcctttgggctttatgttgtatgttactaaccaccttttaccatatttatcttttggcatcattctgggacattgctaaattaaaaaagaaattgtttccacttttttctggagatgttcaactaaaggttgttttgttttgttttttgttttgagacagtctcaccctgacgctcaggctggagtgcagtggtgcaacctcggctcactgcaacctccacctcccgggctcaagccattctcctgcctcagcctcccaagcagctgggattacaggcacccgccaccacgcccagctaatttttttgtattttgagtagagaccgggtttcaccatattggccagtctcgtctggaactcctgacctcagatgatccgcccgcctcagcctcccaaagtgctgggattacaggcatgagccaccacgcccagcgtccaacccactgttggatgaaacttgctgcacgtcatacattttgctgttggcaaacaagtctgaatgttgatttgaagtttggtagtttattactatctattggcagcaaagactgtttattggtatactacaatatgatttaacttttattttggggataaatagtagaaaaaagtgaaacagaatgaaggcaggtgttttttattctaatgatggaataatacagagatactggacgatctctagcagttaattattgtgacccatataaaattatacaggtcacagtataattctctattaccgtttttacaccagtaagtcttagataaactaagcatgcttatgaattatgtatacagttagaatgcattatttttacagaggaacaattgcttgtatgtactaacactgttctcttggcttgcctcaagttctactcattattttatataaaatactattaggctgggcacggtggctcacgcctataatcccagcactttgggaggtggaggctggcggattacttgaagccaggagttcgagaccagcctggccaaaatggtgaaaccccatctctataaaaatacaaaaattagccaggtgtcatgatacatgcctgtaatcccagcttcttgggaggctgaggcacgggaatcgcttgaacccgggaagcacaggttgcagtgagccaagatcatgccactgcacccccagcctgggtgacagagtgcaacactgtctcacaaaacaaaacaaaaacatcagattctgtttgtgatgcctagttgcttacaacctaaacagtgcaatgccttaaggaaatgaaaaggagccataagtagtcatttatatttttattttgaagtgtgctttttctaaactcccagattgacatgatggactgtaagttagtttctctgtttctgtctttgtgcctgtagagtgtacttggcacttacaaattcccagtatccagaaagatgatctgatgaaatcaaattggatggatcttggcagactgtgacactcaattacagccttcactttcagtcaaaaacggacacttggcaaggaggtgcctggttgtttcactaaatgtcacttgtgtgtgtaatattttaaagctttttccccacaggaaattcgggtcataaaatcctgaaaaataattctaggtgggaaaagcattttaggaaatgagagatgtggtgctgcttttcttctctcagagtgctttctcagcaggacactagccctgcctttaagatggggaagttggggcatgtgcctctgctcttactgtctgcagctctgaaggtaggtgctgtcccactcggacaatcgcccaagcagcagtgaccatagttctcttctatgcaagtccccaggagaaggtaaactgtgtggaatggggatgtgttctggttgctgctgaatcccctcttcttaccacagtgcctggcacgttgcacacactcaaatacgtaataatgaacatttattgaaagcagcagttgaagctgaccaatttctggtaccttgtcatgtaaattttagatggtaaggcgcagatgttactttttttgctttttttcttcagcacttgatgaaatttcccaaacatgcagaaatgttgaaagacttgtatagtgaacatctacgacctagaatctgcagtaatattatgttacatttgctttatcacttgatagatgttacttttaatgagacttcaagtttggtttctctaaacaaaatattctaaaataactgaacaactttaatcaatttgtcttaagttctttgggggaacttgggacatttgctttgtaactggaattgcagccctcacgttaagctaattttaaactttgcaaatttgttatgctgaatttcagtcttatttattttgcctgaaggggtattttttgtaatggatttatttgaaggtccttgataaattgtgcagaatattctcgtgttctttttgcacttgataaattatctaatttctgtggtgagaatgtaatttggggcctattttgtttatacaagcttccagaattatgttctcagagggatgaaaaggtgtaatttagcatataggtcactaaattaggagctaagacacattttctcctgactgaccatgggtcaatcagttttgtcttcgtgtccttttccttgtaaagtagaaactagaatttgaaatttaaatattaaataatgggtaacattcattaatgtatgactctattaagaaagacactgtgaatccagggaggattctcataattctgtaaactgtatgacaagctgtggaatgaaatctgacttttgaaaattgaaagacatccagtggtcttatcacaaagcctgcttttcctcagaacttaactattgccatggaatttgtaagcagttatcctaatccatctggactctgaaaatgcatcctttatgagagggagtgaatgcaaagataagggtggggaaacactaatcatgaaaagaatgaaaatcagtgttcagttttaagagcaggttgtattgaaggaagggattaaaggaattatccagatttgaggtggcacatcttccaccactccctgcaccatcagcatgcacggagcgcataaaacaagccctgctcctaatggcagtgaaacctcggatggcctccatcaggtcaatacaactgaattgctgggctgacttaagattgaaggactccattttagtaagtagagaagtgtgacctttctcaacccaggttgtgaatgtggattcacacttatctcaaaaaggcacctggagttttaactttatgtcatgtctcagtactggttgcaaggtatgaccaaaagtgttccttgaatggcacctttttgaatattaatttagaagaaaacatgccagactgacatacttaccccctccgcactgttactacttccttaccagccctatgtactgcatcaatgtctacaagaaagcactcttcattaaaatgaaatatatatattaaaataaaaaaaaaaaaaaaaaaaa

SEQ ID NO: 64 - protein, 522 aa, Homo sapiens; identical to SEQ ID NO: 62

SEQ ID NO: 65 - DNA, 1742 bp, Homo sapiens
tctttgaagcttcaaggctgctgaataatttccttctcccattttgtgcctgcctagctatccagacagagcagctaccctcagctctagctgatactacagacagtacaacagatcaagaagtatggcagtgacaactcgtttgacatggttgcacgaaaagatcctgcaaaatcattttggagggaagcggcttagccttctctataagggtagtgtccatggattccgtaatggagttttgcttgacagatgttgtaatcaagggcctactctaacagtgatttatagtgaagatcatattattggagcatatgcagaagagagttaccaggaaggaaagtatgcttccatcatcctttttgcacttcaagatactaaaatttcagaatggaaactaggactatgtacaccagaaacactgttttgttgtgatgttacaaaatataactccccaactaatttccagatagatggaagaaatagaaaagtgattatggacttaaagacaatggaaaatcttggacttgctcaaaattgtactatctctattcaggattatgaagtttttcgatgcgaagattcactggatgaaagaaagataaaaggggtcattgagctcaggaagagcttactgtctgccttgagaacttatgaaccatatggatccctggttcaacaaatacgaattctgctgctgggtccaattggagctgggaagtccagctttttcaactcagtgaggtctgttttccaagggcatgtaacgcatcaggctttggtgggcactaatacaactgggatatctgagaagtataggacatactctattagagacgggaaagatggcaaatacctgccgtttattctgtgtgactcactggggctgagtgagaaagaaggcggcctgtgcagggatgacatattctatatcttgaacggtaacattcgtgatagataccagtttaatcccatggaatcaatcaaattaaatcatcatgactacattgattccccatcgctgaaggacagaattcattgtgtggcatttgtatttgatgccagctctattcaatacttctcctctcagatgatagtaaagatcaaaagaattcgaagggagttggtaaacgctggtgtggtacatgtggctttgctcactcatgtggatagcatggatttgattacaaaaggtgaccttatagaaatagagagatgtgagcctgtgaggtccaagctagaggaagtccaaagaaaacttggatttgctctttctgacatctcggtggttagcaattattcctctgagtgggagctggaccctgtaaaggatgttctaattctttctgctctgagacgaatgctatgggctgcagatgacttcttagaggatttgccttttgagcaaatagggaatctaagggaggaaattatcaactgtgcacaaggaaaaaaatagatatgtgaaaggttcacgtaaatttcctcacatcacagaagattaaaattcagaaaggagaaaacacagaccaaagagaagtatctaagaccaaagggatgtgttttattaatgtctaggatgaagaaatgcatagaacattgtagtacttgtaaataactagaaataacatgatttagtcataattgtgaaaaataataataatttttcttggatttatgttctgtatctgtgaaaaaataaatttcttataaaactcgggtctaaaaaaaaaaaaaaaaaa

SEQ ID NO: 66 - protein, 444 aa, Homo sapiens
MAVTTRLTWLHEKILQNHFGGKRLSLLYKGSVHGFRNGVLLDRCCNQGPTLTVIYSEDHIIGAYAEESYQEGKYASIILFALQDTKISEWKLGLCTPETLFCCDVTKYNSPTNFQIDGRNRKVIMDLKTMENLGLAQNCTISIQDYEVFRCEDSLDERKIKGVIELRKSLLSALRTYEPYGSLVQQIRILLLGPIGAGKSSFFNSVRSVFQGHVTHQALVGTNTTGISEKYRTYSIRDGKDGKYLPFILCDSLGLSEKEGGLCRDDIFYILNGNIRDRYQFNPMESIKLNHHDYIDSPSLKDRIHCVAFVFDASSIQYFSSQMIVKIKRIRRELVNAGVVHVALLTHVDSMDLITKGDLIEIERCEPVRSKLEEVQRKLGFALSDISVVSNYSSEWELDPVKDVLILSALRRMLWAADDFLEDLPFEQIGNLREEIINCAQGKK

SEQ ID NO: 67 - DNA, 2464 bp, Homo sapiens
actttcctttcccctttcataaaagcacagacctaacagcaccctgggtggaaacctcttcagcatttgcttggaatcagtaagctaaaaacaaaatcaaccgggaccccagcttttcagaactgcagggaaacagccatcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggacgataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactccatctcaaaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgttaaaaaaaaaaa

SEQ ID NO: 68 - protein, 490 aa, Homo sapiens
MSEVTKNSLEKILPQLKCHFTWNLFKEDSVSRDLEDRVCNQIEFLNTEFKATMYNLLAYIKHLDGNNEAALECLRQAEELIQQEHADQAEIRSLVTWGNYAWVYYHLGRLSDAQIYVDKVKQTCKKFSNPYSIEYSELDCEEGWTQLKCGRNERAKVCFEKALEEKPNNPEFSSGLAIAMYHLDNHPEKQFSTDVLKQAIELSPDNQYVKVLLGLKLQKMNKEAEGEQFVEEALEKSPCQTDVLRSAAKFYRRKGDLDKAIELFQRVLESTPNNGYLYHQIGCCYKAKVRQMQNTGESEASGNKEMIEALKQYAMDYSNKALEKGLNPLNAYSDLAEFLETECYQTPFNKEVPDAEKQQSHQRYCNLQKYNGKSEDTAVQHGLEGLSISKKSTDKEEIKDQPQNVSENLLPQNAPNYWYLQGLIHKQNGDLLQAAKCYEKELGRLLRDAPSGIGSIFLSASELEDGSEEMGQGAVSSSPRELLSNSEQLN

SEQ ID NO: 69 - DNA, 2552 bp, Homo sapiens
attttcctcctcccaacgattttaaattagtttcactttccagtttcctcttccttcccctaaaagcaattactcaaaaacggagaaaacatcagctgatgcgtgccctactctcccacccctttatatagttccttcagtatttacttgaggcagacaggaagacttctgaagaacaaatcagcctggtcaccagcttttcggaacagcagagacacagagggcagtcatgagtgaggtcaccaagaattccctggagaaaatccttccacagctgaaatgccatttcacctggaacttattcaaggaagacagtgtctcaagggatctagaagatagagtgtgtaaccagattgaatttttaaacactgagttcaaagctacaatgtacaacttgttggcctacataaaacacctagatggtaacaacgaggcagccctggaatgcttacggcaagctgaagagttaatccagcaagaacatgctgaccaagcagaaatcagaagtctagtcacttggggaaactacgcctgggtctactatcacttgggcagactctcagatgctcagatttatgtagataaggtgaaacaaacctgcaagaaattttcaaatccatacagtattgagtattctgaacttgactgtgaggaagggtggacacaactgaagtgtggaagaaatgaaagggcgaaggtgtgttttgagaaggctctggaagaaaagcccaacaacccagaattctcctctggactggcaattgcgatgtaccatctggataatcacccagagaaacagttctctactgatgttttgaagcaggccattgagctgagtcctgataaccaatacgtcaaggttctcttgggcctgaaactgcagaagatgaataaagaagctgaaggagagcagtttgttgaagaagccttggaaaagtctccttgccaaacagatgtcctccgcagtgcagccaaattttacagaagaaaaggtgacctagacaaagctattgaactgtttcaacgggtgttggaatccacaccaaacaatggctacctctatcaccagattgggtgctgctacaaggcaaaagtaagacaaatgcagaatacaggagaatctgaagctagtggaaataaagagatgattgaagcactaaagcaatatgctatggactattcgaataaagctcttgagaagggactgaatcctctgaatgcatactccgatctcgctgagttcctggagacggaatgttatcagacaccattcaataaggaagtccctgatgctgaaaagcaacaatcccatcagcgctactgcaaccttcagaaatataatgggaagtctgaagacactgctgtgcaacatggtttagagggtttgtccataagcaaaaaatcaactgacaaggaagagatcaaagaccaaccacagaatgtatctgaaaatctgcttccacaaaatgcaccaaattattggtatcttcaaggattaattcataagcagaatggagatctgctgcaagcagccaaatgttatgagaaggaactgggccgcctgctaagggatgccccttcaggcataggcagtattttcctgtcagcatctgagcttgaggatggtagtgaggaaatgggccagggcgcagtcagctccagtcccagagagctcctctctaactcagagcaactgaactgagacagaggaggaaaacagagcatcagaagcctgcagtggtggttgtgacgggtaggacgataggaagacagggggccccaacctgggattgctgagcagggaagctttgcatgttgctctaaggtacatttttaaagagttgttttttggccgggcgcagtggctcatgcctgtaatcccagcactttgggaggccgaggtgggcggatcacgaggtctggagtttgagaccatcctggctaacacagtgaaatcccgtctctactaaaaatacaaaaaattagccaggcgtggtggctggcacctgtagtcccagctacttgggaggctgaggcaggagaatggcgtgaacctggaaggaagaggttgcagtgagccaagattgcgcccctgcactccagcctgggcaacagagcaagactccatctcaaaaaaaaaaaaaaaaaaaaaaaagagttgttttctcatgttcattatagttcattacagttacatagtccgaaggtcttacaactaatcactggtagcaataaatgcttcaggcccacatgatgctgattagttctcagttttcattcagttcacaatataaccaccattcctgccctccctgccaagggtcataaatggtgactgcctaacaacaaaatttgcagtctcatctcattttcatccagacttctggaactcaaagattaacttttgactaaccctggaatatctcttatctcacttatagcttcaggcatgtatttatatgtattcttgatagcaataccataatcaatgtgtattcctgatagtaatgctacaataaatccaaacatttcaactctgttaaaaaaaaaaa

SEQ ID NO: 70 - protein, 490 aa, Homo sapiens; identical to SEQ ID NO: 68

SEQ ID NO: 71 - DNA, 2103 bp, Homo sapiens
tgactagacggccagcctgttaaggtggccccagatattccagcctcagcccagagtcctcctgtgcccctactgcagcaagggtgtctccaagaagggggacctggagtcagcccgtcacacctggtttcctctctgctagggtccctcctcccacagagcactggagggcagctgaggaggagctaccttaaaaaaggaggtgtgtgccagggagctgggtaggagcctggctatatatctgcccagcagcggtactctcgggacagagatggcactgatgcaggaactgtatagcacaccagcctccaggctggactccttcgtggctcagtggctgcagccccaccgggagtggaaggaagaggtgctagacgctgtgcggaccgtggaggagtttctgaggcaggagcatttccaggggaagcgtgggctggaccaggatgtgcgggtgctgaaggtagtcaaggtgggctccttcgggaatggcacggttctcaggagcaccagagaggtggagctggtggcgtttctgagctgtttccacagcttccaggaggcagccaagcatcacaaagatgttctgaggctgatatggaaaaccatgtggcaaagccaggacctgctggacctcgggctcgaggacctgaggatggagcagagagtccccgatgctctcgtcttcaccatccagaccagggggactgcggagcccatcacggtcaccattgtgcctgcctacagagccctggggccttctcttcccaactcccagccaccccctgaggtctatgtgagcctgatcaaggcctgcggtggtcctggaaatttctgcccatccttcagcgagctgcagagaaatttcgtgaaacatcggccaactaagctgaagagcctcctgcgcctggtgaaacactggtaccagcagtatgtgaaagccaggtcccccagagccaatctgccccctctctatgctcttgaacttctaaccatctatgcctgggaaatgggtactgaagaagacgagaatttcatgttggacgaaggcttcaccactgtgatggacctgctcctggagtatgaagtcatctgtatctactggaccaagtactacacactccacaatgcaatcattgaggattgtgtcagaaaacagctcaaaaaagagaggcccatcatcctggatccggccgaccccaccctcaacgtggcagaagggtacagatgggacatcgttgctcagagggcctcccagtgcctgaaacaggactgttgctatgacaacagggagaaccccatctccagctggaacgtgaagagggcacgagacatccacttgacagtggagcagaggggttacccagatttcaacctcatcgtgaacccttatgagcccataaggaaggttaaagagaaaatccggaggaccaggggctactctggcctgcagcgtctgtccttccaggttcctggcagtgagaggcagcttctcagcagcaggtgctccttagccaaatatgggatcttctcccacactcacatctatctgctggagaccatcccctccgagatccaggtcttcgtgaagaatcctgatggtgggagctacgcctatgccatcaaccccaacagcttcatcctgggtctgaagcagcagattgaagaccagcaggggcttcctaaaaagcagcagcagctggaattccaaggccaagtcctgcaggactggttgggtctggggatctatggcatccaagacagtgacactctcatcctctcgaagaagaaaggagaggctctgtttccagccagttagttttctctgggagacttctctgtacatttctgccatgtactccagaactcatcctgtcaatcactctgtcccattgtctactgggaaggtcccaggtcttcaccagttttacaatgagttatcccaggccagacgtggtagctcacacctgtaatcccagaactttgggaggccgaggtgggaggagcgcttgagccgaggagttcaagaccagcctgggtatcacagggagaccccgtctctacaaaataaaaaaataattcactgggaaaaaaaaaaaaaaaaaa

SEQ ID NO: 72 - protein, 514 aa, Homo sapiens
MALMQELYSTPASRLDSFVAQWLQPHREWKEEVLDAVRTVEEFLRQEHFQGKRGLDQDVRVLKVVKVGSFGNGTVLRSTREVELVAFLSCFHSFQEAAKHHKDVLRLIWKTMWQSQDLLDLGLEDLRMEQRVPDALVFTIQTRGTAEPITVTIVPAYRALGPSLPNSQPPPEVYVSLIKACGGPGNFCPSFSELQRNFVKHRPTKLKSLLRLVKHWYQQYVKARSPRANLPPLYALELLTIYAWEMGTEEDENFMLDEGFTTVMDLLLEYEVICIYWTKYYTLHNAIIEDCVRKQLKKERPIILDPADPTLNVAEGYRWDIVAQRASQCLKQDCCYDNRENPISSWNVKRARDIHLTVEQRGYPDFNLIVNPYEPIRKVKEKIRRTRGYSGLQRLSFQVPGSERQLLSSRCSLAKYGIFSHTHIYLLETIPSEIQVFVKNPDGGSYAYAINPNSFILGLKQQIEDQQGLPKKQQQLEFQGQVLQDWLGLGIYGIQDSDTLILSKKKGEALFPAS

SEQ ID NO: 73 - DNA, 1861 bp, Homo sapiens
tgactagacggccagcctgttaaggtggccccagatattccagcctcagcccagagtcctcctgtgcccctactgcagcaagggtgtctccaagaagggggacctggagtcagcccgtcacacctggtttcctctctgctagggtccctcctcccacagagcactggagggcagctgaggaggagctaccttaaaaaaggaggtgtgtgccagggagctgggtaggagcctggctatatatctgcccagcagcggtactctcgggacagagatggcactgatgcaggaactgtatagcacaccagcctccaggctggactccttcgtggctcagtggctgcagccccaccgggagtggaaggaagaggtgctagacgctgtgcggaccgtggaggagtttctgaggcaggagcatttccaggggaagcgtgggctggaccaggatgtgcgggtgctgaaggtagtcaaggtgggctccttcgggaatggcacggttctcaggagcaccagagaggtggagctggtggcgtttctgagctgtttccacagcttccaggaggcagccaagcatcacaaagatgttctgaggctgatatggaaaaccatgtggcaaagccaggacctgctggacctcgggctcgaggacctgaggatggagcagagagtccccgatgctctcgtcttcaccatccagaccagggggactgcggagcccatcacggtcaccattgtgcctgcctacagagccctggggccttctcttcccaactcccagccaccccctgaggtctatgtgagcctgatcaaggcctgcggtggtcctggaaatttctgcccatccttcagcgagctgcagagaaatttcgtgaaacatcggccaactaagctgaagagcctcctgcgcctggtgaaacactggtaccagcaggcccatcatcctggatccggccgaccccaccctcaacgtggcagaagggtacagatgggacatcgttgctcagagggcctcccagtgcctgaaacaggactgttgctatgacaacagggagaaccccatctccagctggaacgtgaagagggcacgagacatccacttgacagtggagcagaggggttacccagatttcaacctcatcgtgaacccttatgagcccataaggaaggttaaagagaaaatccggaggaccaggggctactctggcctgcagcgtctgtccttccaggttcctggcagtgagaggcagcttctcagcagcaggtgctccttagccaaatatgggatcttctcccacactcacatctatctgctggagaccatcccctccgagatccaggtcttcgtgaagaatcctgatggtgggagctacgcctatgccatcaaccccaacagcttcatcctgggtctgaagcagcagattgaagaccagcaggggcttcctaaaaagcagcagcagctggaattccaaggccaagtcctgcaggactggttgggtctggggatctatggcatccaagacagtgacactctcatcctctcgaagaagaaaggagaggctctgtttccagccagttagttttctctgggagacttctctgtacatttctgccatgtactccagaactcatcctgtcaatcactctgtcccattgtctactgggaaggtcccaggtcttcaccagttttacaatgagttatcccaggccagacgtggtagctcacacctgtaatcccagaactttgggaggccgaggtgggaggagcgcttgagccgaggagttcaagaccagcctgggtatcacagggagaccccgtctctacaaaataaaaaaataattcactgggaaaaaaaaaaaaaaaaaa

SEQ ID NO: 74 - protein, 255 aa, Homo sapiens
MALMQELYSTPASRLDSFVAQWLQPHREWKEEVLDAVRTVEEFLRQEHFQGKRGLDQDVRVLKVVKVGSFGNGTVLRSTREVELVAFLSCFHSFQEAAKHHKDVLRLIWKTMWQSQDLLDLGLEDLRMEQRVPDALVFTIQTRGTAEPITVTIVPAYRALGPSLPNSQPPPEVYVSLIKACGGPGNFCPSFSELQRNFVKHRPTKLKSLLRLVKHWYQQAHHPGSGRPHPQRGRRVQMGHRCSEGLPVPETGLLL

SEQ ID NO: 75 - DNA, 3099 bp, Homo sapiens
aaatattcttgcttgagtaaaccacagtaagaataaggaagtagtgactgagtgccttgccagtacagcagatgctagaacataatgtagcattactttccccagggtttattgttatgtaagttcttgttcagcttcctttgttttctttcacttctgagaatttaactttcgtttctcactcagctcctgtggggaaactcatttgtggagaccagccctctggcttggtgagtgaatctggtttacaccggctcctgccctgccttcactcttctcccctgattcaagactcctctgctttggactgaagcactgcaggagtttgtgaccaagaacttcaagagtcaagacagaaggaagccaagggagcagtgcaatggatttctcagtaaaggtagacatagagaaggaggtgacctgccccatctgcctggagctcctgacagaacctctgagcctagattgtggccacagcttctgccaagcctgcatcactgcaaagatcaaggagtcagtgatcatctcaagaggggaaagcagctgtcctgtgtgtcagaccagattccagcctgggaacctccgacctaatcggcatctggccaacatagttgagagagtcaaagaggtcaagatgagcccacaggaggggcagaagagagatgtctgtgagcaccatggaaaaaaactccagatcttctgtaaggaggatggaaaagtcatttgctgggtttgtgaactgtctcaggaacaccaaggtcaccaaacattccgcataaacgaggtggtcaaggaatgtcaggaaaagctgcaggtagccctgcagaggctgataaaggaggatcaagaggctgagaagctggaagatgacatcagacaagagagaaccgcctggaagatcgagagacagaagattctgaaagggttcaatgaaatgagagtcatcttggacaatgaggagcagagagagctgcaaaagctggaggaaggtgaggtgaatgtgctggataacctggcagcagctacagaccagctggtccagcagaggcaggatgccagcacgctcatctcagatctccagcggaggttgaggggatcgtcagtagagatgctgcaggatgtgattgacgtcatgaaaaggagtgaaagctggacattgaagaagccaaaatctgtttccaagaaactaaagagtgtattccgagtaccagatctgagtgggatgctgcaagttcttaaagagctgacagatgtccagtactactgggtggacgtgatgctgaatccaggcagtgccacttcgaatgttgctatttctgtggatcagagacaagtgaaaactgtacgcacctgcacatttaagaattcaaatccatgtgatttttctgcttttggtgtcttcggctgccaatatttctcttcggggaaatattactgggaagtagatgtgtctggaaagattgcctggatcctgggcgtacacagtaaaataagtagtctgaataaaaggaagagctctgggtttgcttttgatccaagtgtaaattattcaaaagtttactccagatatagacctcaatatggctactgggttataggattacagaatacatgtgaatataatgcttttgaggactcctcctcttctgatcccaaggttttgactctctttatggctgtgcctccctgtcgtattggggttttcctagactatgaggcaggcattgtctcatttttcaatgtcacaaaccacggagcactcatctacaagttctctggatgtcgcttttctcgacctgcttatccgtatttcaatccttggaactgcctagtccccatgactgtgtgcccaccgagctcctgagtgttctcattcctttacccacttctgcatagtagcccttgtgctgagactcagattctgcacctgagttcatctctactgagaccatctcttcctttctttccccttcttttacttagaatgtctttgtattcatttgctagggcttccatagcaaagcatcatagattgctgatttaaactgtaattgtattgccgtactgtgggctggaaatcccaaatctagattccagcagagttggttctttctgaggtctgcaaggaagggctctgttccatgcctctctccttggcttgtagaaggcatcttgtccctatgactcttcacattgtctttatgtacatctctgtgcccaagttttccctttttattaagacaccagtcatactggctcagggcccaccgctaatgccttaatgaaatcattttaacattatattctctacaaagaccttatttccaaataagataatatttggaggtattgggaataaaaactccaacatataaatttgaggaaggcacgatttcactcataacaatcttaccctttcttgcaagagatgcttgtacattattttcctaataccttggtttcactagtagtaaacattattattttttttatatttgcaaaggaaacatatctaatccttcctatagaaagaacagtattgctgtaattccttttcttttcttcctcatttcctctgccccttaaaagattgaagaaagagaaacttgtcaactcatatccacgttatctagcaaagtacataagaatctatcactaagtaatgtatccttcagaatgtgttggtttaccagtgacaccccatattcatcacaaaattaaagcaagaagtccatagtaatttatttgctaatagtggatttttaatgctcagagtttctgaggtcaaattttatcttttcacttacaagctctatgatcttaaataatttacttaatgtattttggtgtattttcctcaaattaatattggtgttcaagactatatctaattcctctgatcactttgagaaacaaacttttattaaatgtaaggcacttttctatgaattttaaatataaaaataaatattgttctgattattactgaaaagatgtcagccatttcaatgtcttgggaaacaattttttgtttttgttctgttttctttttgcttcaataaaacaatagctggctctaaaaaaaaaaaaaaaaaaaaa

SEQ ID NO: 76 - protein, 494 aa, Homo sapiens
MDFSVKVDIEKEVTCPICLELLTEPLSLDCGHSFCQACITAKIKESVIISRGESSCPVCQTRFQPGNLRPNRHLANIVERVKEVKMSPQEGQKRDVCEHHGKKLQIFCKEDGKVICWVCELSQEHQGHQTFRINEVVKECQEKLQVALQRLIKEDQEAEKLEDDIRQERTAWKIERQKILKGFNEMRVILDNEEQRELQKLEEGEVNVLDNLAAATDQLVQQRQDASTLISDLQRRLRGSSVEMLQDVIDVMKRSESWTLKKPKSVSKKLKSVFRVPDLSGMLQVLKELTDVQYYWVDVMLNPGSATSNVAISVDQRQVKTVRTCTFKNSNPCDFSAFGVFGCQYFSSGKYYWEVDVSGKIAWILGVHSKISSLNKRKSSGFAFDPSVNYSKVYSRYRPQYGYWVIGLQNTCEYNAFEDSSSSDPKVLTLFMAVPPCRIGVFLDYEAGIVSFFNVTNHGALIYKFSGCRFSRPAYPYFNPWNCLVPMTVCPPSS

SEQ ID NO: 77 - DNA, 3111 bp, Homo sapiens
aaatattcttgcttgagtaaaccacagtaagaataaggaagtagtgactgagtgccttgccagtacagcagatgctagaacataatgtagcattactttccccagggtttattgttatgtaagttcttgttcagcttcctttgttttctttcacttctgagaatttaactttcgtttctcactcagctcctgtggggaaactcatttgtggagaccagccctctggcttggtgagtgaatctggtttacaccggctcctgccctgccttcactcttctcccctgattcaagactcctctgctttggactgaagcactgcaggagtttgtgaccaagaacttcaagagtcaagacagaaggaagccaagggagcagtgcaatggatttctcagtaaaggtagacatagagaaggaggtgacctgccccatctgcctggagctcctgacagaacctctgagcctagattgtggccacagcttctgccaagcctgcatcactgcaaagatcaaggagtcagtgatcatctcaagaggggaaagcagctgtcctgtgtgtcagaccagattccagcctgggaacctccgacctaatcggcatctggccaacatagttgagagagtcaaagaggtcaagatgagcccacaggaggggcagaagagagatgtctgtgagcaccatggaaaaaaactccagatcttctgtaaggaggatggaaaagtcatttgctgggtttgtgaactgtctcaggaacaccaaggtcaccaaacattccgcataaacgaggtggtcaaggaatgtcaggaaaagctgcaggtagccctgcagaggctgataaaggaggatcaagaggctgagaagctggaagatgacatcagacaagagagaaccgcctggaagaattatatccagatcgagagacagaagattctgaaagggttcaatgaaatgagagtcatcttggacaatgaggagcagagagagctgcaaaagctggaggaaggtgaggtgaatgtgctggataacctggcagcagctacagaccagctggtccagcagaggcaggatgccagcacgctcatctcagatctccagcggaggttgaggggatcgtcagtagagatgctgcaggatgtgattgacgtcatgaaaaggagtgaaagctggacattgaagaagccaaaatctgtttccaagaaactaaagagtgtattccgagtaccagatctgagtgggatgctgcaagttcttaaagagctgacagatgtccagtactactgggtggacgtgatgctgaatccaggcagtgccacttcgaatgttgctatttctgtggatcagagacaagtgaaaactgtacgcacctgcacatttaagaattcaaatccatgtgatttttctgcttttggtgtcttcggctgccaatatttctcttcggggaaatattactgggaagtagatgtgtctggaaagattgcctggatcctgggcgtacacagtaaaataagtagtctgaataaaaggaagagctctgggtttgcttttgatccaagtgtaaattattcaaaagtttactccagatatagacctcaatatggctactgggttataggattacagaatacatgtgaatataatgcttttgaggactcctcctcttctgatcccaaggttttgactctctttatggctgtgcctccctgtcgtattggggttttcctagactatgaggcaggcattgtctcatttttcaatgtcacaaaccacggagcactcatctacaagttctctggatgtcgcttttctcgacctgcttatccgtatttcaatccttggaactgcctagtccccatgactgtgtgcccaccgagctcctgagtgttctcattcctttacccacttctgcatagtagcccttgtgctgagactcagattctgcacctgagttcatctctactgagaccatctcttcctttctttccccttcttttacttagaatgtctttgtattcatttgctagggcttccatagcaaagcatcatagattgctgatttaaactgtaattgtattgccgtactgtgggctggaaatcccaaatctagattccagcagagttggttctttctgaggtctgcaaggaagggctctgttccatgcctctctccttggcttgtagaaggcatcttgtccctatgactcttcacattgtctttatgtacatctctgtgcccaagttttccctttttattaagacaccagtcatactggctcagggcccaccgctaatgccttaatgaaatcattttaacattatattctctacaaagaccttatttccaaataagataatatttggaggtattgggaataaaaactccaacatataaatttgaggaaggcacgatttcactcataacaatcttaccctttcttgcaagagatgcttgtacattattttcctaataccttggtttcactagtagtaaacattattattttttttatatttgcaaaggaaacatatctaatccttcctatagaaagaacagtattgctgtaattccttttcttttcttcctcatttcctctgccccttaaaagattgaagaaagagaaacttgtcaactcatatccacgttatctagcaaagtacataagaatctatcactaagtaatgtatccttcagaatgtgttggtttaccagtgacaccccatattcatcacaaaattaaagcaagaagtccatagtaatttatttgctaatagtggatttttaatgctcagagtttctgaggtcaaattttatcttttcacttacaagctctatgatcttaaataatttacttaatgtattttggtgtattttcctcaaattaatattggtgttcaagactatatctaattcctctgatcactttgagaaacaaacttttattaaatgtaaggcacttttctatgaattttaaatataaaaataaatattgttctgattattactgaaaagatgtcagccatttcaatgtcttgggaaacaattttttgtttttgttctgttttctttttgcttcaataaaacaatagctggctctaaaaaaaaaaaaaaaaaaaaa

SEQ ID NO: 78 - protein, 498 aa, Homo sapiens
MDFSVKVDIEKEVTCPICLELLTEPLSLDCGHSFCQACITAKIKESVIISRGESSCPVCQTRFQPGNLRPNRHLANIVERVKEVKMSPQEGQKRDVCEHHGKKLQIFCKEDGKVICWVCELSQEHQGHQTFRINEVVKECQEKLQVALQRLIKEDQEAEKLEDDIRQERTAWKNYIQIERQKILKGFNEMRVILDNEEQRELQKLEEGEVNVLDNLAAATDQLVQQRQDASTLISDLQRRLRGSSVEMLQDVIDVMKRSESWTLKKPKSVSKKLKSVFRVPDLSGMLQVLKELTDVQYYWVDVMLNPGSATSNVAISVDQRQVKTVRTCTFKNSNPCDFSAFGVFGCQYFSSGKYYWEVDVSGKIAWILGVHSKISSLNKRKSSGFAFDPSVNYSKVYSRYRPQYGYWVIGLQNTCEYNAFEDSSSSDPKVLTLFMAVPPCRIGVFLDYEAGIVSFFNVTNHGALIYKFSGCRFSRPAYPYFNPWNCLVPMTVCPPSS

SEQ ID NO: 79 - DNA, 4396 bp, Homo sapiens
gcaaggacacacccacagcttacaccattggctgctgtttagctcccttatataacactgtcttggggtttaaacgtaactgaaaatccacaagacagaatagccagatctcagaggagcctggctaagcaaaaccctgcagaacggctgcctaatttacagcaaccatgagtacaaatggtgatgatcatcaggtcaaggatagtctggagcaattgagatgtcactttacatgggagttatccattgatgacgatgaaatgcctgatttagaaaacagagtcttggatcagattgaattcctagacaccaaatacagtgtgggaatacacaacctactagcctatgtgaaacacctgaaaggccagaatgaggaagccctgaagagcttaaaagaagctgaaaacttaatgcaggaagaacatgacaaccaagcaaatgtgaggagtctggtgacctggggcaactttgcctggatgtattaccacatgggcagactggcagaagcccagacttacctggacaaggtggagaacatttgcaagaagctttcaaatcccttccgctatagaatggagtgtccagaaatagactgtgaggaaggatgggccttgctgaagtgtggaggaaaaaattatgaacgggccaaggcctgctttgaaaaggtgcttgaagtggaccctgaaaaccctgaatccagcgctgggtatgcgatctctgcctatcgcctggatggctttaaattagccacaaaaaatcacaagccattttctttgcttcccctaaggcaggctgtccgcttaaatccagacaatggatatattaaggttctccttgccctgaagcttcaggatgaaggacaggaagctgaaggagaaaagtacattgaagaagctctagccaacatgtcctcacagacctatgtctttcgatatgcagccaagttttaccgaagaaaaggctctgtggataaagctcttgagttattaaaaaaggccttgcaggaaacacccacttctgtcttactgcatcaccagatagggctttgctacaaggcacaaatgatccaaatcaaggaggctacaaaagggcagcctagagggcagaacagagaaaagctagacaaaatgataagatcagccatatttcattttgaatctgcagtggaaaaaaagcccacatttgaggtggctcatctagacctggcaagaatgtatatagaagcaggcaatcacagaaaagctgaagagaattttcaaaaattgttatgcatgaaaccagtggtagaagaaacaatgcaagacatacatttccactatggtcggtttcaggaatttcaaaagaaatctgacgtcaatgcaattatccattatttaaaagctataaaaatagaacaggcatcattaacaagggataaaagtatcaattctttgaagaaattggttttaaggaaacttcggagaaaggcattagatctggaaagcttgagcctccttgggttcgtctacaaattggaaggaaatatgaatgaagccctggagtactatgagcgggccctgagactggctgctgactttgagaactctgtgagacaaggtccttaggcacccagatatcagccactttcacatttcatttcattttatgctaacatttactaatcatcttttctgcttactgttttcagaaacattataattcactgtaatgatgtaattcttgaataataaatctgacaaaatattagttgtgttcaacaattagtgaaacagaatgtgtgtatgcatgtaagaaagagaaatcatttgtatgagtgctatgtagtagagaaaaaatgttagttaactttgtaggaaataaaacattggacttacactaaatgtttaattcattcattttattgtgaaataaaaataaaatccttagctcctccaccaactgaacagaccctcttggccaaggagaccccagaaaccttaaaaactaagtttcccaaccatgacaagatgagagatcattcacacctcattatattccctcccttgctaactgccattggactttttccactgagttaaacagaaacccatggaaaacaaagaacagaagactcactccttggctgacttcacctagctcactccacgtagcgccacagccagactcccctcccctcttgcggtttccacatgacaactgatcagccttccctcctgataagtgaccactgcccacagactggttctggccagtccatggaggctgcacacagggtgcctctatgtcctttgtttcaccttttgatatagaaaggctaattttgctgtattttaatgttaagtctccaccacagagtgaacacagaatgcatgtgacatacatgtttacataccactattgtgtgactgcccctcatgaatattcatagccccccataacctgttaactatgtgtgtctagccaatccaccaaccataaaacttctgtaataccctcccttcctccaagagcctgcttttggttgctgtggtaggctctgcttcccaggctgcaggttgcaggagaggaggctgcagtggctcacgcctgtaatctcagcacttcgatgggacgaggcaggcagatcacctgaacccaggagttcgagagcagccttggcaatggcaaaaccaaccgtctctacaaaaaatgcaaaaacttagctgggtgtggtggcatgcacctgtagcttcagttccagctactcaggaggctgaggtgagtggactgctggagccagggagttcgaggctgcagtgtcgagatcttgccactgcactccattctggatgatagaacgagaccccatctcaaaaaaaaaaaaagttctctccaattgtatatagcttgtgattttatgtcaacactatcaataaatagctttcagtgcaagaaaccaaaaatactgtaataaacaggcacatattcttcccaaacctcatgcagtttacaatctagtgagagacacagatagcagtacagagtcaattaaaggttagttttcttcatgaagatgttttaattttaattcaatgtgaaagggttccaaggagtttatcttgttttatgccattttatttgaagcactacttactaagtcatttgctgatattaatctagttaaatcaagaaatattacatgaaaatgttgctaaatcagagatcatgggtaacaatcacctttgattatgaataatcatattttattgaaaggcaaggcacaacaaataataagaaggaaaaaataaataagcaatgttattgatctttcattctgtatatgttttggggggaatatactagtttcttttagtggctgtaacaaattaccacaaacttggtgacttaaaatttcacagatttactctttcttacagttctggaggtcagaagtctgaaatgggtttcaatgagccaaagtcaaggtattgatgacgctacactcctccggaggctctaggcagatagccttttccagcttccagaggctgcctgaattctttcatccatcttaaaaaccaacagtgtagtagcctcaaatctctctctctgcttccttcttcacatctccttctctcctctgactcttttgcctctttcttctaaggacgcaccaggtccacctgcataatccagaataattgccccatccgcaaatccttaatttaataacatctgcaaagtcccttttgctatgtaaagtagcatgttcacaggttctggagacttggccatggatacgattgcggggggggcattattcttaccacagagcaccccaagaaaatctccaaattttgggcttccaatccattttgcttcaattatttaatatttttactccttccagtagatactgatttcatccattgcccttaagaaggtaggacagagattatggcacatctcacattaaatgctatattttcgttggaaatacattttttgcttcaacttttattttaaattcaagggtacatgtgcaggatgttcaggtttgttacacaggtaaacgtgtgccatggcggtttgctgaacagatcatcccatcaccaacagatcatcccattgagaggtgaagccggctgggcttctgggttgggtggggacttggagaacttttctgtctagctaaagtattgtaaaatggaccagtcaacactctgtaaaatggaccaatcagctctctgtaaaatggaccaatcagcaggatgtgggtggggccaagtaagggaataaaagcaggccacccgagctggcagcggcaacccgctcgggtccccttccatgctgtggaagttttgttctttcgctctttcaataaatcttgctgctgctcaaaaaaaaaaaaaaaaaa

SEQ ID NO: 80 - protein, 478 aa, Homo sapiens
MSTNGDDHQVKDSLEQLRCHFTWELSIDDDEMPDLENRVLDQIEFLDTKYSVGIHNLLAYVKHLKGQNEEALKSLKEAENLMQEEHDNQANVRSLVTWGNFAWMYYHMGRLAEAQTYLDKVENICKKLSNPFRYRMECPEIDCEEGWALLKCGGKNYERAKACFEKVLEVDPENPESSAGYAISAYRLDGFKLATKNHKPFSLLPLRQAVRLNPDNGYIKVLLALKLQDEGQEAEGEKYIEEALANMSSQTYVFRYAAKFYRRKGSVDKALELLKKALQETPTSVLLHHQIGLCYKAQMIQIKEATKGQPRGQNREKLDKMIRSAIFHFESAVEKKPTFEVAHLDLARMYIEAGNHRKAEENFQKLLCMKPVVEETMQDIHFHYGRFQEFQKKSDVNAIIHYLKAIKIEQASLTRDKSINSLKKLVLRKLRRKALDLESLSLLGFVYKLEGNMNEALEYYERALRLAADFENSVRQGP

SEQ ID NO: 81 - DNA, 3499 bp, Homo sapiens
cgcggacccggccggcccaggcccgcgcccgccgcggccctgagaggccccggcaggtcccggcccggcggcggcagccatggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctgatgtgttcacctgggcagatcccccaaatccggaagtcaaagttctcatggtcagaagttctcatggtgcacgagtcctcagcactctgccggcagtgggggtgggggcccatgcccgcgggggagagaaggaggtggccctgctgttctaggctctgtgggcataggcaggcagagtggaaccctgcctccatgccagcatctgggggcaaggaaggctggcatcatccagtgaggaggctggcgcatgttgggaggctgctggctgcacagacccgtgaggggaggagaggggctgctgtgcaggggtgtggagtagggagctggctcccctgagagccatgcagggcgtctgcagcccaggcctctggcagcagctctttgcccatctctttggacagtggccaccctgcacaatggggccgacgaggcctagggccctcctacctgcttacaatttggaaaagtgtggccgggtgcggtggctcacgcctgtaatcccagcactttgggaggccaaggcaggaggatcgctggagcccagtaggtcaagaccagccagggcaacatgatgagaccctgtctctgccaaaaaattttttaaactattagcctggcgtggtagcgcacgcctgtggtcccagctgctggggaggctgaagtaggaggatcatttatgcttgggaggtcgaggctgcagtgagtcatgattgtatgactgcactccagcctgggtgacagagcaagaccctgtttcaaaaagaaaaaccctgggaaaagtgaagtatggctgtaagtctcatggttcagtcctagcaagaagcgagaattctgagatcctccagaaagtcgagcagcacccacctccaacctcgggccagtgtcttcaggctttactggggacctgcgagctggcctaatgtggtggcctgcaagccaggccatccctgggcgccacagacgagctccgagccaggtcaggcttcggaggccacaagctcagcctcaggcccaggcactgattgtggcagaggggccactacccaaggtctagctaggcccaagacctagttacccagacagtgagaagcccctggaaggcagaaaagttgggagcatggcagacagggaagggaaacattttcagggaaaagacatgtatcacatgtcttcagaagcaagtcaggtttcatgtaaccgagtgtcctcttgcgtgtccaaaagtagcccagggctgtagcacaggcttcacagtgattttgtgttcagccgtgagtcacactacatgcccccgtgaagctgggcattggtgacgtccaggttgtccttgagtaataaaaacgtatgttgcaataaaaaaaaaaaaaaaaaa

SEQ ID NO: 82 - protein, 682 aa, Homo sapiens
MAGGPGPGEPAAPGAQHFLYEVPPWVMCRFYKVMDALEPADWCQFAALIVRDQTELRLCERSGQRTASVLWPWINRNARVADLVHILTHLQLLRARDIITAWHPPAPLPSPGTTAPRPSSIPAPAEAEAWSPRKLPSSASTFLSPAFPGSQTHSGPELGLVPSPASLWPPPPSPAPSSTKPGPESSVSLLQGARPFPFCWPLCEISRGTHNFSEELKIGEGGFGCVYRAVMRNTVYAVKRLKENADLEWTAVKQSFLTEVEQLSRFRHPNIVDFAGYCAQNGFYCLVYGFLPNGSLEDRLHCQTQACPPLSWPQRLDILLGTARAIQFLHQDSPSLIHGDIKSSNVLLDERLTPKLGDFGLARFSRFAGSSPSQSSMVARTQTVRGTLAYLPEEYIKTGRLAVDTDTFSFGVVVLETLAGQRAVKTHGARTKYLKDLVEEEAEEAGVALRSTQSTLQAGLAADAWAAPIAMQIYKKHLDPRPGPCPPELGLGLGQLACCCLHRRAKRRPPMTQENSYVSSTGRAHSGAAPWQPLAAPSGASAQAAEQLQRGPNQPVESDESLGGLSAALRSWHLTPSCPLDPAPLREAGCPQGDTAGESSWGSGPGSRPTAVEGLALGSSASSSSEPPQIIINPARQKMVQKLALYEDGALDSLQLLSSSSLPGLGLEQDRQGPEESDEFQS

SEQ ID NO: 83 - DNA, 3352 bp, Homo sapiens
cgcggacccggccggcccaggcccgcgcccgccgcggccctgagaggccccggcaggtcccggcccggcggcggcagccatggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctggtgtacgagaggctagagaagctgcaggcagtggtggcgggggtgcccgggcattcggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctgatgtgttcacctgggcagatcccccaaatccggaagtcaaagttctcatggtcagaagttctcatggtgcacgagtcctcagcactctgccggcagtgggggtgggggcccatgcccgcgggggagagaaggaggtggccctgctgttctaggctctgtgggcataggcaggcagagtggaaccctgcctccatgccagcatctgggggcaaggaaggctggcatcatccagtgaggaggctggcgcatgttgggaggctgctggctgcacagacccgtgaggggaggagaggggctgctgtgcaggggtgtggagtagggagctggctcccctgagagccatgcagggcgtctgcagcccaggcctctggcagcagctctttgcccatctctttggacagtggccaccctgcacaatggggccgacgaggcctagggccctcctacctgcttacaatttggaaaagtgtggccgggtgcggtggctcacgcctgtaatcccagcactttgggaggccaaggcaggaggatcgctggagcccagtaggtcaagaccagccagggcaacatgatgagaccctgtctctgccaaaaaattttttaaactattagcctggcgtggtagcgcacgcctgtggtcccagctgctggggaggctgaagtaggaggatcatttatgcttgggaggtcgaggctgcagtgagtcatgattgtatgactgcactccagcctgggtgacagagcaagaccctgtttcaaaaagaaaaaccctgggaaaagtgaagtatggctgtaagtctcatggttcagtcctagcaagaagcgagaattctgagatcctccagaaagtcgagcagcacccacctccaacctcgggccagtgtcttcaggctttactggggacctgcgagctggcctaatgtggtggcctgcaagccaggccatccctgggcgccacagacgagctccgagccaggtcaggcttcggaggccacaagctcagcctcaggcccaggcactgattgtggcagaggggccactacccaaggtctagctaggcccaagacctagttacccagacagtgagaagcccctggaaggcagaaaagttgggagcatggcagacagggaagggaaacattttcagggaaaagacatgtatcacatgtcttcagaagcaagtcaggtttcatgtaaccgagtgtcctcttgcgtgtccaaaagtagcccagggctgtagcacaggcttcacagtgattttgtgttcagccgtgagtcacactacatgcccccgtgaagctgggcattggtgacgtccaggttgtccttgagtaataaaaacgtatgttgcaataaaaaaaaaaaaaaaaaa

SEQ ID NO: 84 - protein, 633 aa, Homo sapiens
MAGGPGPGEPAAPGAQHFLYEVPPWVMCRFYKVMDALEPADWCQFAALIVRDQTELRLCERSGQRTASVLWPWINRNARVADLVHILTHLQLLRARDIITAWHPPAPLPSPGTTAPRPSSIPAPAEAEAWSPRKLPSSASTFLSPAFPGSQTHSGPELGLVPSPASLWPPPPSPAPSSTKPGPESSVSLLQGARPFPFCWPLCEISRGTHNFSEELKIGEGGFGCVYRAVMRNTVYAVKRLKENADLEWTAVKQSFLTEVEQLSRFRHPNIVDFAGYCAQNGFYCLVYGFLPNGSLEDRLHCQTQACPPLSWPQRLDILLGTARAIQFLHQDSPSLIHGDIKSSNVLLDERLTPKLGDFGLARFSRFAGSSPSQSSMVARTQTVRGTLAYLPEEYIKTGRLAVDTDTFSFGVVVLETLAGQRAVKTHGARTKYLVYERLEKLQAVVAGVPGHSEAASCIPPSPQENSYVSSTGRAHSGAAPWQPLAAPSGASAQAAEQLQRGPNQPVESDESLGGLSAALRSWHLTPSCPLDPAPLREAGCPQGDTAGESSWGSGPGSRPTAVEGLALGSSASSSSEPPQIIINPARQKMVQKLALYEDGALDSLQLLSSSSLPGLGLEQDRQGPEESDEFQS

SEQ ID NO: 85 - DNA, 3589 bp, Homo sapiens
cgcggacccggccggcccaggcccgcgcccgccgcggccctgagaggccccggcaggtcccggcccggcggcggcagccatggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggtgtacgagaggctagagaagctgcaggcagtggtggcgggggtgcccgggcattcggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctgatgtgttcacctgggcagatcccccaaatccggaagtcaaagttctcatggtcagaagttctcatggtgcacgagtcctcagcactctgccggcagtgggggtgggggcccatgcccgcgggggagagaaggaggtggccctgctgttctaggctctgtgggcataggcaggcagagtggaaccctgcctccatgccagcatctgggggcaaggaaggctggcatcatccagtgaggaggctggcgcatgttgggaggctgctggctgcacagacccgtgaggggaggagaggggctgctgtgcaggggtgtggagtagggagctggctcccctgagagccatgcagggcgtctgcagcccaggcctctggcagcagctctttgcccatctctttggacagtggccaccctgcacaatggggccgacgaggcctagggccctcctacctgcttacaatttggaaaagtgtggccgggtgcggtggctcacgcctgtaatcccagcactttgggaggccaaggcaggaggatcgctggagcccagtaggtcaagaccagccagggcaacatgatgagaccctgtctctgccaaaaaattttttaaactattagcctggcgtggtagcgcacgcctgtggtcccagctgctggggaggctgaagtaggaggatcatttatgcttgggaggtcgaggctgcagtgagtcatgattgtatgactgcactccagcctgggtgacagagcaagaccctgtttcaaaaagaaaaaccctgggaaaagtgaagtatggctgtaagtctcatggttcagtcctagcaagaagcgagaattctgagatcctccagaaagtcgagcagcacccacctccaacctcgggccagtgtcttcaggctttactggggacctgcgagctggcctaatgtggtggcctgcaagccaggccatccctgggcgccacagacgagctccgagccaggtcaggcttcggaggccacaagctcagcctcaggcccaggcactgattgtggcagaggggccactacccaaggtctagctaggcccaagacctagttacccagacagtgagaagcccctggaaggcagaaaagttgggagcatggcagacagggaagggaaacattttcagggaaaagacatgtatcacatgtcttcagaagcaagtcaggtttcatgtaaccgagtgtcctcttgcgtgtccaaaagtagcccagggctgtagcacaggcttcacagtgattttgtgttcagccgtgagtcacactacatgcccccgtgaagctgggcattggtgacgtccaggttgtccttgagtaataaaaacgtatgttgcaataaaaaaaaaaaaaaaaaa

SEQ ID NO: 86 - protein, 712 aa, Homo sapiens
MAGGPGPGEPAAPGAQHFLYEVPPWVMCRFYKVMDALEPADWCQFAALIVRDQTELRLCERSGQRTASVLWPWINRNARVADLVHILTHLQLLRARDIITAWHPPAPLPSPGTTAPRPSSIPAPAEAEAWSPRKLPSSASTFLSPAFPGSQTHSGPELGLVPSPASLWPPPPSPAPSSTKPGPESSVSLLQGARPFPFCWPLCEISRGTHNFSEELKIGEGGFGCVYRAVMRNTVYAVKRLKENADLEWTAVKQSFLTEVEQLSRFRHPNIVDFAGYCAQNGFYCLVYGFLPNGSLEDRLHCQTQACPPLSWPQRLDILLGTARAIQFLHQDSPSLIHGDIKSSNVLLDERLTPKLGDFGLARFSRFAGSSPSQSSMVARTQTVRGTLAYLPEEYIKTGRLAVDTDTFSFGVVVLETLAGQRAVKTHGARTKYLKDLVEEEAEEAGVALRSTQSTLQAGLAADAWAAPIAMQIYKKHLDPRPGPCPPELGLGLGQLACCCLHRRAKRRPPMTQVYERLEKLQAVVAGVPGHSEAASCIPPSPQENSYVSSTGRAHSGAAPWQPLAAPSGASAQAAEQLQRGPNQPVESDESLGGLSAALRSWHLTPSCPLDPAPLREAGCPQGDTAGESSWGSGPGSRPTAVEGLALGSSASSSSEPPQIIINPARQKMVQKLALYEDGALDSLQLLSSSSLPGLGLEQDRQGPEESDEFQS

SEQ ID NO: 87 - DNA, 3469 bp, Homo sapiens
gcccctggctagagaagccgcagcccgcagtgtccgacccagtcgtcccgcgccggagccggccccgtagcgtgccatggcctgctacatctaccagctgccctcctgggtgctggacgacctgtgccgcaacatggacgcgctcagcgagtgggactggatggagttcgcctcctacgtgatcacagacctgacccagctgcggaagatcaagtccatggagcgggtgcagggtgtgagcatcacgcgggagctgctgtggtggtggggcatgcggcaggccaccgtccagcaacttgtggacctcctgtgccgcctggagctctaccgggctgcccagatcatcctgaactggaaaccggctcctgaaatcaggtgtcccattccagccttccctgactctgtgaagccagaaaagcctttggcagcttctgtaagaaaggctgaggatgaacaggaagaggggcagcctgtgaggatggccacctttccaggcccagggtcctctccagccagagcccaccagccggcctttctccagcctcctgaagaagatgcccctcattccttgagaagcgacctccccacttcgtctgattcaaaggacttcagcacctccattcctaagcaggaaaaacttttgagcttggctggagacagccttttctggagtgaggcagacgtggtccaggcaaccgatgacttcaatcaaaaccgcaaaatcagccaggggacctttgctgacgtctacagagggcacaggcacgggaagccattcgtcttcaagaagctcagagagacagcctgttcaagtccaggatcaatcgaaagattcttccaggcagagttgcagatttgtcttagatgctgccaccccaatgtcttacctgtgctgggcttctgtgctgcaagacagtttcacagcttcatctacccctacatggcaaatggttccctacaggacagactgcagggtcagggtggctcggaccccctcccctggccccagcgtgtcagcatctgctcagggctgctctgtgccgtcgagtacctgcatggtctggagatcatccacagcaacgtcaagagctctaatgtcttgctggaccaaaatctcacccccaaacttgctcacccaatggctcatctgtgtcctgtcaacaaaaggtcaaaatacaccatgatgaagactcacctgctccggacgtcagccgcgtatctgccagaggatttcatccgggtggggcagctgacaaagcgagtggacatcttcagctgtggaatagtgttggccgaggtcctcacgggcatccctgcaatggataacaaccgaagcccggtttacctgaaggacttactcctcagtgatattccaagcagcaccgcctcgctctgctccaggaagacgggcgtggagaacgtgatggcaaaggagatctgccagaagtacctggagaagggcgcagggaggcttccggaggactgcgccgaggccctggccacggctgcctgcctgtgcctgcggaggcgtaacaccagcctgcaggaggtgtgtggctctgtggctgctgtggaagagcggctccgaggtcgggagacgttgctcccttggagtgggctttctgagggtacaggctcttcttccaacaccccagaggaaacagacgacgttgacaattccagccttgatgcctcctcctccatgagtgtggcaccctgggcaggggctgccaccccacttctccccacagagaatggggaaggaaggctgcgggtcatcgtgggaagggaggctgactcctcctctgaggcctgtgttggcctggagcctccccaggatgttacagaaacttcgtggcaaattgagatcaatgaggccaaaaggaaactgatggagaatattctgctctacaaagaggaaaaagtggacagcattgagctctttggcccctgatgaccggaacacagctgaggacccttgtcctcagttggaaagatgagcatcagatcaagaaaaaggtctgaggcagaatccaagatctgccaggaaacacacaacaaaacatctgctgtcctgggtgggagggaaacttcatttcactggaatgagttgggagagaaaggccctcagcttttagagacacaaaaatccatgaagtctcttcctttctgggctttgttagtcagagcaggggatcagaggagactgaagcagaaaccctgcacacgggcccaggatgtggctgattttgtggttccggggagtatgtgatgataatcacccccagcagattccattacctcagcagctcttgttcccccgccactggcagttctgcaatgccatagcattttccagagctaagatctctgggttgtatttgctgacagcctgcaagcttgcatgctctgaaagattttttttagtttttaatttttttgtagagatggggtctcgctttgttggcgcaatcctcccacctcagactcccaaagtgctggaattacagttgggagccactgtgcctggcctggaagactttcaacttgtgtctcagtgcagttcttgactcacctctctgggcctcaggttctacaaatgccagacacctagcgaagagctctgcaggctttccactgcctgtattggaaatcttgcaattcacataattattcagtcactgcctggtacctttatcttcccatcccactaatgttagtgttttttaatggagcttttattctgagaatatgtgtttgtctgtttgtttgttttttgagacagagtctcactttgtcacccaggctggagtgcagtggcacgatctcagctcactgcaagctctgcctctcaggttcaagtgattctcctgcctcagcctcctgagtagatgggactgtaggcacctgccactatgcctggctaatttttgtgtttttagtagagacagggtttcaccatattggccaggctggtctcgaactactgacctcgtgatctgcccgccttggcctatcaaagtgttgggattacaggcttgagccaccgcacccggccgagaatatgtgttgttatttatgactggattatgaagaatcaggagaatgcatttcatgtctgattctgctgctaattaagtcaatcatttaatttttgggacctcagtttctttgtaagtaaaataacacctgcttgttcttcatccctgggctgttgggaggaacagatgagacagtggctatagaagcacttggaaaatgcacttgtcctgttttgtaaaataaaaaggtattaaatgtgtatttctgccatgtacctaatgattattcagtgcgtatatatctgaaaagtcatgttgcaaatctttctgtgaaacagatgctattttaaattcactgggagaaatatcctatttaaagtaatctatagtaatttctttttatataataaaaatatatttgtaaagtc

SEQ ID NO: 88 - protein, 625 aa, Homo sapiens
MACYIYQLPSWVLDDLCRNMDALSEWDWMEFASYVITDLTQLRKIKSMERVQGVSITRELLWWWGMRQATVQQLVDLLCRLELYRAAQIILNWKPAPEIRCPIPAFPDSVKPEKPLAASVRKAEDEQEEGQPVRMATFPGPGSSPARAHQPAFLQPPEEDAPHSLRSDLPTSSDSKDFSTSIPKQEKLLSLAGDSLFWSEADVVQATDDFNQNRKISQGTFADVYRGHRHGKPFVFKKLRETACSSPGSIERFFQAELQICLRCCHPNVLPVLGFCAARQFHSFIYPYMANGSLQDRLQGQGGSDPLPWPQRVSICSGLLCAVEYLHGLEIIHSNVKSSNVLLDQNLTPKLAHPMAHLCPVNKRSKYTMMKTHLLRTSAAYLPEDFIRVGQLTKRVDIFSCGIVLAEVLTGIPAMDNNRSPVYLKDLLLSDIPSSTASLCSRKTGVENVMAKEICQKYLEKGAGRLPEDCAEALATAACLCLRRRNTSLQEVCGSVAAVEERLRGRETLLPWSGLSEGTGSSSNTPEETDDVDNSSLDASSSMSVAPWAGAATPLLPTENGEGRLRVIVGREADSSSEACVGLEPPQDVTETSWQIEINEAKRKLMENILLYKEEKVDSIELFGP

SEQ ID NO: 89 - RNA, 22 nt, Artificial Sequence
  Anti-sense miR-146a
aacccauggaauucaguucuca

SEQ ID NO: 90 - DNA, 275 bp, Homo sapiens
gtttacctgaaggacttactcctcagaacgtgatggcaaaggagatctgcgagatctgccagaagtacctggagatgcggaggcgtaacaccagcctgcacgaggtcgggagacgttgctccctttgctcccttggagtgggctttctgatgggctttctgagggtacaggctctagggtacaggctcttcttccaacaccaccccagaggaaacagacgacgttctccatgagtgtggcaccctgggcaacttcgtggcaaattgagatcaatg

SEQ ID NO: 91 - DNA, 275 bp, Homo sapiens
aggagaatgcatttcatgtctgatttcatgtctgattctgctgctaattaatcatttaatttttgggacctcagtaataacacctgcttgttcttcatccttcttcatccctgggctgttgggaggggaggaacagatgagacagtggctgacagtggctatagaagcacttggaaatgcacttgtcctgttttgtaaaagtatttctgccatgtacctaatgatgtacctaatgattattcagtgcgtagaaaagtcatgttgcaaatctttct

SEQ ID NO: 92 - DNA, 22 bp, Artificial Sequence
  MiR-146a prob
aacccatggaattcagttctca

SEQ ID NO: 93 - DNA, 21 bp, Artificial Sequence
  MiR-146a 5′primer
gtgccgaggagggatctagaa

SEQ ID NO: 94 - DNA, 20 bp, Artificial Sequence
  MiR-146a 3′primer
cctgcacgctaaccctctct

SEQ ID NO: 95 - DNA, 23 bp, Artificial Sequence
  MiR-146a 5′primer
cgactggagcacgaggacactga

SEQ ID NO: 96 - DNA, 26 bp, Artificial Sequence
  MiR-146a 5′primer
ggacactgacatggactgaaggagta

SEQ ID NO: 97 - DNA, 25 bp, Artificial Sequence
  MiR-146a 5′primer
ttcagctgggatatctctgtcatcg

SEQ ID NO: 98 - DNA, 23 bp, Artificial Sequence
  MiR-146a 5′primer
gggcttgaggacctggagagagt

SEQ ID NO: 99 - DNA, 24 bp, Artificial Sequence
  MiR-146a 3′primer
gctgaggatacacatcggcttttc

SEQ ID NO: 100 - DNA, 25 bp, Artificial Sequence
  MiR-146a 3′primer
ctcctcgttgtgctactgtctcctg

SEQ ID NO: 101 - DNA, 25 bp, Artificial Sequence
  MiR-146a 3′primer
gctgtcaacgatacgctacgtaacg

SEQ ID NO: 102 - DNA, 23 bp, Artificial Sequence
  MiR-146a 3′primer
cgctacgtaacggcatgacagtg

SEQ ID NO: 103 - DNA, 275 bp, Homo sapiens
agcgttacagccctgcatttgagattaagttgccttgattctgacatttgtggtgtgccgcaatgagagtcaatcaatctctattgacagcctgcttcagccttctgtccttggaacagtcatataagataggtcctactgcaaaccaccctccatatttccgtaccatttacaagacatctttttaaaccactggaggaggattttacttatctgtgtattcaccaagcatacttgccattacttttccctctccaacatcacattcactttaa

SEQ ID NO: 104 - DNA, 275 bp, Homo sapiens
tactccctcagcatcagagcatgcactcttccctgaccttcacgaagggattcacgaagggatggctctccagtcggtcccgtagcacacagttacagtgatactgctatcattcttcgctaattgcacagcacctgcagcattgtaactcccaggccttggacatttagtgacttttagtgactgttagccggtcccttggttcactgtcattgtgtttcccagtttcttcttgtttccctgattatgagagcttccattgttctgttaagtct

SEQ ID NO: 105 - DNA, 275 bp, Homo sapiens
tatgtgtgtggtacctgttgtgtccaagatacgctttccatttgatgatggagttgacatggaggcagtgcttgcttgctttgttcgcctatcatctggctggccacatgaggctgtcaagcaaaagtgtagttgagtagctggttggccaaatcacgctggaaccttgggcaaggagagtgcctggatttcatgtcagtaatagggtaccatctaggtcagtttggaacaaaatcctctccttgtggaaggaaatatcccatgcagtttgttga

SEQ ID NO: 106 - DNA, 275 bp, Homo sapiens
tccctaacctacacccatagattacccaaggtttcagtgtactagttttgtttataccctcaatttctggcctttttctggcctttggctattttagcatgttaagcactgctttcagttttaataatatccaccttgaggggtcgctgcggatgttacttaatgtggcttctctgtggcttctctaatgtagtttctttagtttctttgattaccgactacacaagtaccatgtgatttaattctccatattctccattcctccaatgtaactc

SEQ ID NO: 107 - DNA, 275 bp, Homo sapiens
agaaaaccacacttctcataccttcttccccaaagccagaagatgcacaatccttccaaggtccaccgtgattaatgattaacatccacagcgagacctcaccctcttcttgaactggtgctgtcgctgtctgggcttcatagcattcgcgccaagtgcctgaacatctgggcccatctgggccctgattctgggcatccgggcatcctcatgaccattggattcatcctgttactggtattcggctctgtgcactccactgtgcaatgctggcc

SEQ ID NO: 108 - DNA, 275 bp, Homo sapiens
tcaacaccctcttcttgaactggtgttcttgaactggtgctgtctgggctacagtctaccatattatgttacagatacagataatacaggaaaaacggggcaacctttgcactccactgtgcaatacctttgcactccactgtgcaatgctgcactccactgtgcaatgctggcccccacacacctgtctacagtgtcatcacacacctgtctacagtgtcattccacacctgtctacagtgtcattcaacacctgtctacagtgtcattcaata

SEQ ID NO: 109 - DNA, 275 bp, Homo sapiens
ggtcttcgctggacaccatgaatcaatgaatcacactgtccaaaccttctttctctcctgtcaacagtggccagcagccccccaactatgagatgctcaatgttcaacaccctcttcatgaacccattcgcctactccgtgaagtctagggccaagtgcctgaacatctgggcccatctgggccctgattctgggcatcccatgaccattctgctcatcgtcatcgcatcactgaggccaggagctctgctcctcacacgcttttctacaatggc

SEQ ID NO: 110 - DNA, 275 bp, Homo sapiens
cagctgcgctacacggaggaactgctgcgctacacggaggaactgctgcgcccggcgcatgggcaagtgcaaggtccccatcttcgacttcagagtcttcgctgtgccgagtgcacctagagggctgcacctagagggcacgcagcgtgaacgcagcgtgagggtgtgtcttccccaacagcctctatgacgacatcgagtatgacgacatcgagtgcttccttagacatcgagtgcttccttatggagccgagtgcttccttatggagctggag

SEQ ID NO: 111 - DNA, 275 bp, Homo sapiens
tggcgggcaacgaattccaggtgtcagctccatgtcggtgtcagagctgatgaaggcgcagatcacccagaagattgctggtggtggacaaatgcgacgagtggacaaatgcgacgaacctctgaggaataacaagggccgcagcagcacagcaagtgagcgggctggagggtgtgagggtgtgcaggacgacctgttctctggctgaccttcgaggggaagcccccctgagcaccgtgttcatgaatctagcaccgtgttcatgaatctgcgcc

SEQ ID NO: 112 - DNA, 275 bp, Homo sapiens
gttgaggatctcttactctctaagcgccacggaattaacccgagcaggcatctcacctcatcagcagtgaccagtccaaagtggtcagggtggcctctggccctggccaggattgctacagttgtttggaggagttgtggccatggcggccaagatgatgtccgcggcggccattccaatgggggtggagttgcctcggggactctccggattgaccaagttcattctgccattgcggctgtcattgcgagagccaactatcccaaatatacctg

SEQ ID NO: 113 - DNA, 275 bp, Homo sapiens
ttattagtgcgctgtgaggtctccatctccacccgctttgacatgggtagttcggagggagggttttcagccacgtgcaggggtatagcttgccctcactttgccctcacttgctcaaaaacaactccttgccctgttctcaataacatggaggtcacttactatttcttcctgtcacacgttccatgctttttcagaaagttgcagcagctattttgccagttagccagttagtatacctctttgttgttttgttgtactttcttgggcttttg

SEQ ID NO: 114 - DNA, 275 bp, Homo sapiens
tgccagctctattcaatacttctccaatacttctcctctcagatgatagtctctttctgacatctcggtggttagggtggttagcaattattcctctgagtcctctgagtgggagctggaccctgagctggaccctgtaaaggatgttctaggatgttctaattctttctgctcttctaattctttctgctctgagacgagctctgagacgaatgctatgggctggaatgctatgggctgcagatgacttctgcagatgacttcttagaggattt

SEQ ID NO: 115 - DNA, 250 bp, Homo sapiens
cctgctaagggatgccccttcaggcggatgccccttcaggcataggcagtgcataggcagtattttcctgtcagcttcctgtcagcatctgagcttgaggagtgaggaaatgggccagggcgcagagtcccagagagctcctctctaactagctcctctctaactcagagcaactgaagcctgcagtggtggttgtgacggcccaacctgggattgctgagcagggaagctttgcatgttgctctaaggt

SEQ ID NO: 116 - DNA, 275 bp, Homo sapiens
aattccaaggccaagtcctgcaggagtcctgcaggactggttgggtctgggttgggtctggggatctatggcatccaagacagtgacactctcatcctctacactctcatcctctcgaagaagaaaggctctgtttccagccagttagtttgggagacttctctgtacatttctgatttctgccatgtactccagaactcccagaactcatcctgtcaatcactctcccaggtcttcaccagttttacaacaatgagttatcccaggccagacgt

SEQ ID NO: 117 - DNA, 275 bp, Homo sapiens
aatttcgtgaaacatcggccaactatcctgcgcctggtgaaacactggtaccccaccctcaacgtggcagaagggatgggacatcgttgctcagagggccgacaacagggagaaccccatctccaggcacgagacatccacttgacagtgggagcagaggggttacccagatttccccagatttcaacctcatcgtgaactcatcgtgaacccttatgagcccattgtccttccaggttcctggcagtgaatcctgatggtgggagctacgccta

SEQ ID NO: 118 - DNA, 275 bp, Homo sapiens
atttcactcataacaatcttaccctcaatcttaccctttcttgcaagagatttcctaataccttggtttcactagtcctctgccccttaaaagattgaaggaaacttgtcaactcatatccacgtaactcatatccacgttatctagcaatgttggtttaccagtgacaccccatgacaccccatattcatcacaaaatttgaggtcaaattttatcttttcactttcacttacaagctctatgatcttagactatatctaattcctctgatcac

SEQ ID NO: 119 - DNA, 275 bp, Homo sapiens
aaaaagcccacatttgaggtggctctggctcatctagacctggcaagaatatttctactatggtcggtttcaggagacgtcaatgcaattatccattatttctggaaagcttgagcctccttgggcctccttgggttcgtctataaattgtgaagccctggagtactatgagcgggagaactctgtgagacaaggtccttcaaggtccttaggcacccagatatcccagatatcagccactttcacattttttactaatcatcttttctgcttac

SEQ ID NO: 120 - DNA, 275 bp, Homo sapiens
tccgagccaggtcaggcttcggagggtggcagaggggccactacccaaggcccaaggtctagctaggcccaagacccaagacctagttacccagacagtgcatgtatcacatgtcttcagaagcatcatgtaaccgagtgtcctcttgcggtcctcttgcgtgtccaaaagtagcggctgtagcacaggcttcacagtgagtgattttgtgttcagccgtgagtctcagccgtgagtcacactacatgccgtgacgtccaggttgtccttgagta

SEQ ID NO: 121 - DNA, 275 bp, Homo sapiens
aaaaggaggcctcctatgacccaggtcctacgtgtccagcactggcagagggcagcgccatcaggagccagtgcctgacgagagcctaggcggcctctcttgcgctcctggcacttgactccaagacagccgtggaaggactggcccttggcagctctgcatcatcgtcgtcagatcgtcagagccaccgcagattatcatgcccgacagaagatggtccagaagtcagagctgatgtgttcacctgggctgatgtgttcacctgggcagatccc